(19) United States
(12) Patent Application Publication
(10) Pub. No.: US 2026/0152509 A1
(43) Pub. Date: Jun. 4, 2026

(54) DELIVERY OF THERAPEUTIC ALKALOID COMPOUNDS

(71) Applicant: SENSORIUM THERAPEUTICS, INC., Boston, MA (US)

(72) Inventor: Jacob M. Hooker, Winchester, MA (US)

(21) Appl. No.: 18/705,639

(22) PCT Filed: Oct. 28, 2022
(86) PCT No.: PCT/US2022/048128

§ 371 Date: Apr. 29, 2024

Related U.S. Application Data
(60) Provisional application No. 63/273,688, filed on Oct. 29, 2021.
(60) Provisional application No. 63/352,812, filed on Jun. 16, 2022.

(51) Int. Cl. C07D 491/113; C07D 495/10; C07D 497/10

(52) U.S. Cl. CPC C07D 491/113; C07D 495/10; C07D 497/10

(57) ABSTRACT

Disclosed are compounds that can be converted to mesembrenone under biologically relevant conditions, such as acid hydrolysis at body temperature; and related methods of preparing and using these compounds. Stable preparations of isolated mesembrenone stereoisomers are also provided.

### RELATED APPLICATIONS

This application claims the benefit of priority to U.S. Provisional Patent Application Nos. 63/273,688, filed Oct. 29, 2021; and 63/352,812, filed Jun. 16, 2022.

### TECHNICAL FIELD

The present disclosure relates to the field of medicine, including the discovery of novel alkaloid compounds useful for inhibiting phosphodiesterase-4 (PDE4).

### BACKGROUND

Plants of the genus Sceletium contain indole alkaloids having biological activity useful in treating mental health conditions such as mild to moderate depression. Natural extracts of Sceletium tortuosum, an indigenous herb of South Africa also referred to as “kougoed”, “channa” or “kanna,” can contain the pharmacologically active alkaloids. Mesembrine and mesembrenol are pharmacologically active alkaloids present in Sceletium tortuosum extracts used for treatment of anxiety, stress and mental health conditions.

Natural products obtained from plants of the genus Sceletium contain varying amounts of (−) mesembrine and (+)/(−) mesembrenone. The structure of mesembrine, also known as 3a-(3,4-dimethoxyphenyl)-octahydro-1-methyl-6H-indol-6-one, has been reported by Popelak et al., Naturwiss. 47,156 (1960), and the configuration by P W Jeffs et al., J. Am. Chem. Soc. 91, 3831 (1969). Naturally occurring (−) mesembrine from Sceletium tortuosum has been reported as having serotonin (5-HT) uptake inhibitory activity useful in treating mental health conditions such as mild to moderate depression. Naturally occurring (+)/(−) mesembrenone from Sceletium tortuosum is reported as a potent selective serotonin reuptake inhibitor (Ki=27 nM).

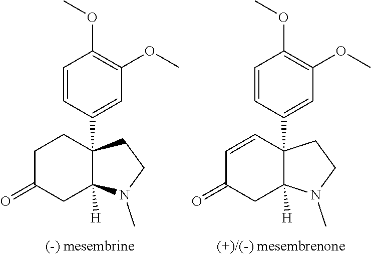 [SMILES: COc1ccc([C@@]23CCC(=O)C[C@@H]2N(C)CC3)cc1OC ; COc1ccc([C@@]23C=CC(=O)C[C@@H]2N(C)CC3)cc1OC]

An analysis of a standardized commercial extract of Sceletium tortuosum was reported in 2011 (obtained as a product under the tradename, Zembrin®) as having 0.35-0.45% total alkaloids, with mesembrenone and mesembrenol comprising ≥60%, and mesembrine contributing <20% (See Harvey et al., “Pharmacological actions of the South African medicinal and functional food plant Sceletium tortuosum and its principal alkaloids,” Journal of Ethnopharmacology 137 (2011) 1124-11292011 and Murbach et. al., “A toxicological safety assessment of a standardized extract of Sceletium tortuosum (Zembrin®) in rats, “Food and Chemical Toxicology 74 (2014) 190-199). The extract gave >80% inhibition at serotonin (5-HT) transporter with potency of the isolated alkaloids at the 5-HT transporter reported as shown in Table A below (Harvey et al., 2011). Referring to the data in Table A, concentration-dependent inhibition was found, with mesembrine being the more active compound (i.e., 20 times more potent than mesembrenone and 87 times more active than mesembrenol) in the 5-HT transporter assay. A toxicological safety assessment of this standardized extract was subsequently reported in 2014 (Murbach et al., 2014).

TABLE A
Summary of analysis of the concentration response curves of alkaloids
on binding to the 5-HT transporter (Harvey et al., 2011)

5-HT transporter (SERT)

| Compound | Ki (nM) | nH |
|---|---|---|
| Mesembrine | 1.4 | 1.0 |
| Mesembrenone | 27 | 1.0 |

However, bioactive plant extracts for therapeutic consumption can vary widely both seasonally and between different Sceletium tortuosum plants, and fail to provide a sufficiently reproducible and stable phytochemical profile of desired biologically active components. Plants of the genus Sceletium and extracts thereof can vary widely in terms of the total alkaloid content, as well as the chemistry and relative concentrations of individual Sceletium plant derived alkaloids. In addition, mesembrine is unstable under a variety of conditions that can occur during extraction from plant material, as well as during storage and formulation of the extract. For example, mesembrine has been reported to be unstable under conditions of fermentation, exposure to light, exposure to heat, and in an aqueous medium.

The therapeutic use of mesembrine and mesembrenone has been limited by the variability and instability of these compounds content in natural extract products and the instability and pharmacokinetic profile of these compounds as obtained from natural products.

There remains an unmet need for pharmaceutical compositions comprising higher purity, predictable, stable and reproducible forms of therapeutic alkaloid compounds such as mesembrenone. In addition, there is a need for oral pharmaceutical compositions providing pure therapeutic alkaloid compositions having desired pharmacokinetic properties upon administration. Finally, there is an unmet need for isolated and stabilized forms of (+) mesembrenone, and pharmaceutical compositions comprising markedly different properties than the naturally occurring compositions obtained from plant extracts.

### SUMMARY

Described are prodrug compounds that, when administered orally or intraveneously to a subject, convert to mesembrenone in vivo. Remarkably, the compounds allow for sustained release of mesembrenone thereby extending exposure of mesembrenone in the brain compared to a subject receiving an equivalent oral or intravenous dose of mesembrenone itself. The sustained release and extended brain exposure to mesembrenone will address recognized therapeutic shortcomings attributed to the pharmacokinetics of mesembrenone. The prodrug compounds provide improved duration of action of mesembrenone for enhanced therapeutic benefit.

Described herein are compounds of formula (I):

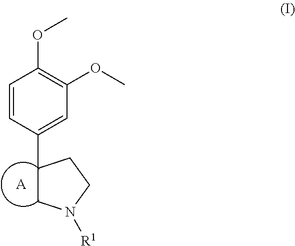

or a pharmaceutically acceptable salt thereof, wherein
   R¹ is H or C₁-C₇ alkyl; and
   ring A is

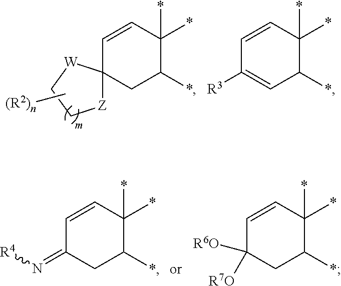

wherein * denotes the attachment points of ring A to the compound of formula (I), and wherein
   each of W and Z is independently O, NH, or S;
   each R² is independently C₁-C₃ alkyl, C₂-C₆ alkenyl, —COOH, —CONH₂, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol;
   m is 1 or 2;
   n is 0, 1, 2, or 3;
   R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy;
   R⁴ is —OR⁵ or —N(R⁵)₂;
   each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, -C(O)N(H)-phenyl, —C(O)N(H)-5- to 7-membered heteroaryl, and —C(O)N(H)-C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, -COOH, —S(O)₂CH₃, —C(O)C₁-C₆ alkyl, or —C(O)OC₁-C₆ alkyl; and each of R⁶ and R⁷ is independently C₁-C₃ alkyl.

In certain embodiments, the compound is of formula (I-1):

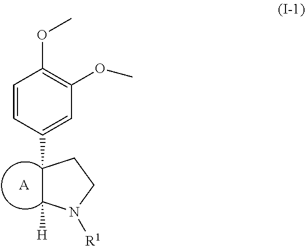

or a pharmaceutically acceptable salt thereof, wherein
   R¹ is H or C₁-C₇ alkyl; and
   ring A is

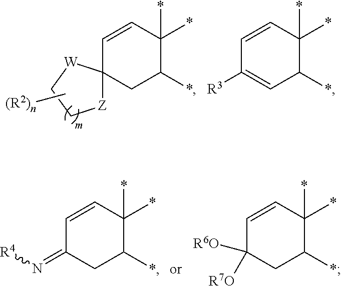

wherein * denotes the attachment points of ring A to the compound of formula (I), and wherein
   each of W and Z is independently O, NH, or S;
each R² is independently C₁-C₃ alkyl, C₂-C₆ alkenyl, —COOH, —CONH₂, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol;
m is 1 or 2;
n is 0, 1, 2, or 3;
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy;
R⁴ is —OR⁵ or —N(R⁵)₂; and
each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl; and
each of R⁶ and R⁷ is independently C₁-C₃ alkyl.

In certain embodiments, the compound is of formula (I-1):

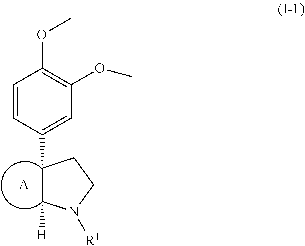

or a pharmaceutically acceptable salt thereof, wherein
   R¹ is H or C₁-C₇ alkyl; and
   ring A is

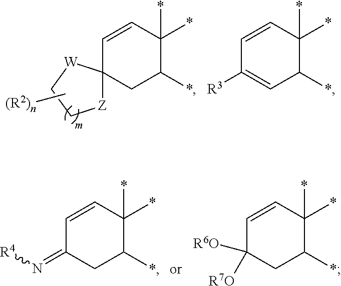

wherein * denotes the attachment points of ring A to the compound of formula (I), and wherein
each of W and Z is independently O, NH, or S;
each R² is independently C₁-C₃ alkyl, C₂-C₆ alkenyl, —COOH, —CONH₂, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol;
m is 1 or 2;
n is 0, 1, 2, or 3;
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl;
each of R⁶ and R⁷ is independently C₁-C₃ alkyl; and
the compound of formula (I-1) has the absolute stereochemistry shown.

In certain embodiments, the compound of formula (I) is a compound of formula (IIb):

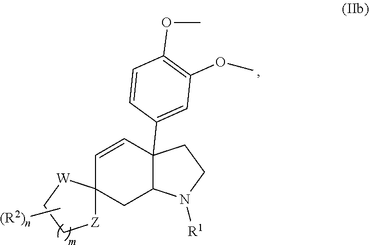

or a pharmaceutically acceptable salt thereof, wherein R¹, R², m, n, W, and Z are as defined herein.

In certain embodiments, the compound is of formula (IIb-1):

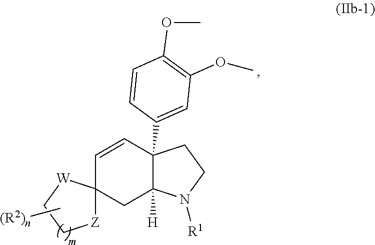

or a pharmaceutically acceptable salt thereof wherein R¹, R², m, n, W, and Z are as defined herein; and the compound of formula (IIb-1) has the absolute stereochemistry shown.

In certain embodiments, the compound of formula (I) is a compound of formula (IVb):

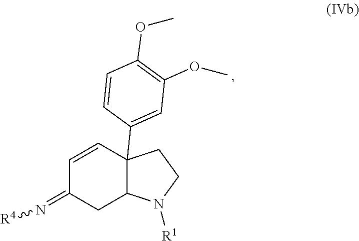

or a pharmaceutically acceptable salt thereof, wherein R¹ and R⁴ are as defined herein.

In certain embodiments, the compound of formula (I) is a compound of formula of formula (IIIb):

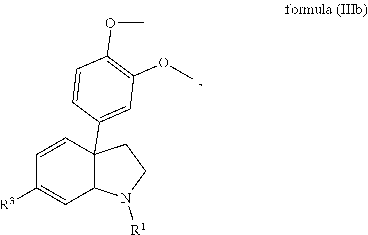

or a pharmaceutically acceptable salt thereof, wherein R¹ and R³ are as defined herein.

In certain embodiments, the compound of formula (I) is a compound of formula (IIIb-1):

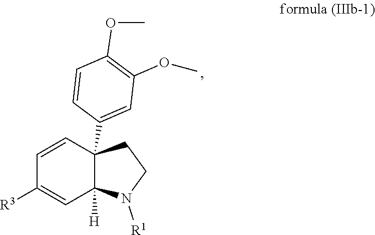

or a pharmaceutically acceptable salt thereof, wherein R¹ and R³ are as defined herein; and the compounds of formula (IIIb-1) have the absolute stereochemistry shown.

In certain embodiments, the compound is of formula (IVb-1):

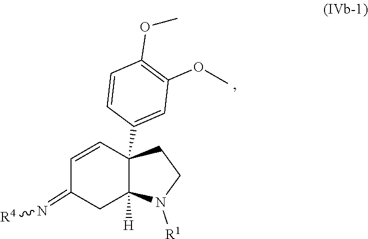

or a pharmaceutically acceptable salt thereof, wherein R¹ and R⁴ are as defined herein.

In certain embodiments, the compound is of formula (IA):

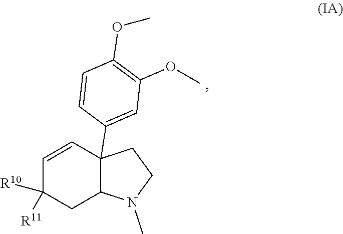

or a pharmaceutically acceptable salt thereof, and R¹⁰ and R¹¹ are as defined herein, for example as a biologically labile moiety selected to provide in vivo conversion of a compound of Formula (IA) to mesembrenone.

In certain embodiments, the compound is of formula (V):

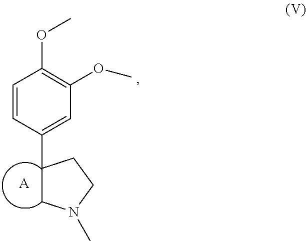

or a pharmaceutically acceptable salt thereof,
wherein
ring A is

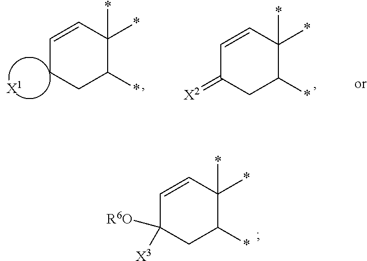

wherein * denotes the attachment points of ring A to the compound of formula (I), and wherein
R⁶ is C₁-C₃ alkyl;
X¹ is >L-R¹⁴, wherein L is absent or a linker and where > denotes the two single bonds to the cyclohexane ring such that the ring containing X¹ forms a C₃-C₆ alkyl ring, which is optionally substituted by C₁-C₃ alkanol;
X² is =L-R¹⁴, wherein L is absent or a linker;
X³ is -L-R¹⁴, wherein L is absent or a linker; and
R¹⁴ comprises a generally recognized as safe (GRAS) compound.

In some embodiments, the compound is of formula (V-1):

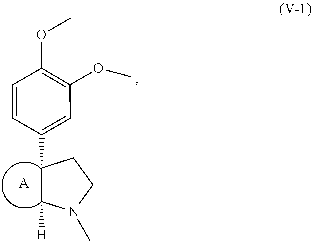

or a pharmaceutically acceptable salt thereof,
wherein
ring A is

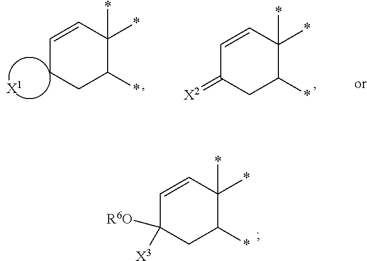

wherein * denotes the attachment points of ring A to the compound of formula (I), and wherein
R⁶ is C₁-C₃ alkyl;
X¹ is >L-R¹⁴, wherein L is absent or a linker and where > denotes the two single bonds to the cyclohexane ring such that the ring containing X¹ forms a C₃-C₆ alkyl ring, which is optionally substituted by C₁-C₃ alkanol;
X² is =L-R¹⁴, wherein L is absent or a linker;
X³ is -L-R¹⁴, wherein L is absent or a linker; and
R¹⁴ comprises a generally recognized as safe (GRAS) compound.

In some embodiments, the compound is selected from the group consisting of

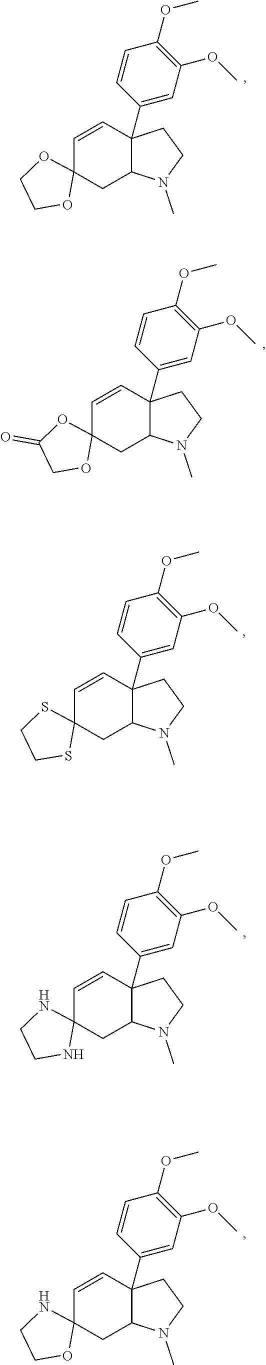 [SMILES: COc1ccc(C23C=CC4(CC2N(C)CC3)OCCO4)cc1OC ; COc1ccc(C23C=CC4(CC2N(C)CC3)OCC(=O)O4)cc1OC ; COc1ccc(C23C=CC4(CC2N(C)CC3)SCCS4)cc1OC ; COc1ccc(C23C=CC4(CC2N(C)CC3)NCCN4)cc1OC ; COc1ccc(C23C=CC4(CC2N(C)CC3)NCCO4)cc1OC]

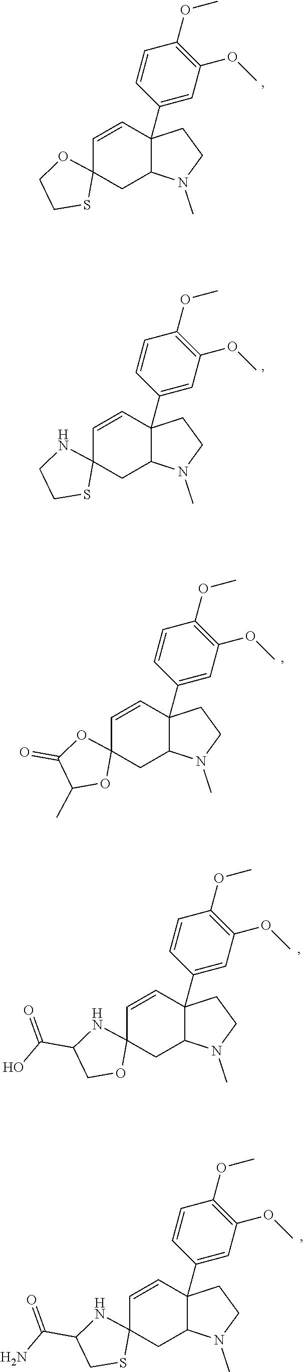 [SMILES: COc1ccc(C23C=CC4(CC2N(C)CC3)OCCS4)cc1OC ; COc1ccc(C23C=CC4(CC2N(C)CC3)NCCS4)cc1OC ; COc1ccc(C23C=CC4(CC2N(C)CC3)OC(=O)C(C)O4)cc1OC ; COc1ccc(C23C=CC4(CC2N(C)CC3)NC(C(=O)O)CO4)cc1OC ; COc1ccc(C23C=CC4(CC2N(C)CC3)NC(C(N)=O)CS4)cc1OC]

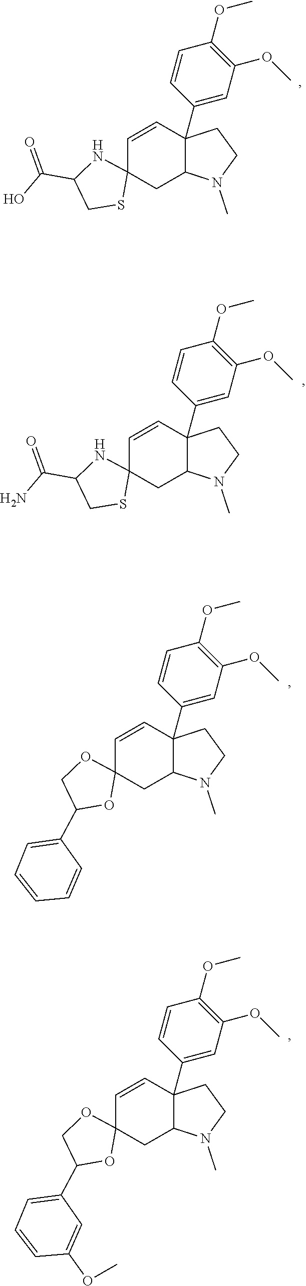 [SMILES: COc1ccc(C23C=CC4(CC2N(C)CC3)NC(C(=O)O)CS4)cc1OC ; COc1ccc(C23C=CC4(CC2N(C)CC3)NC(C(N)=O)CS4)cc1OC ; COc1ccc(C23C=CC4(CC2N(C)CC3)OCC(c2ccccc2)O4)cc1OC ; COc1cccc(C2COC3(C=CC4(c5ccc(OC)c(OC)c5)CCN(C)C4C3)O2)c1]

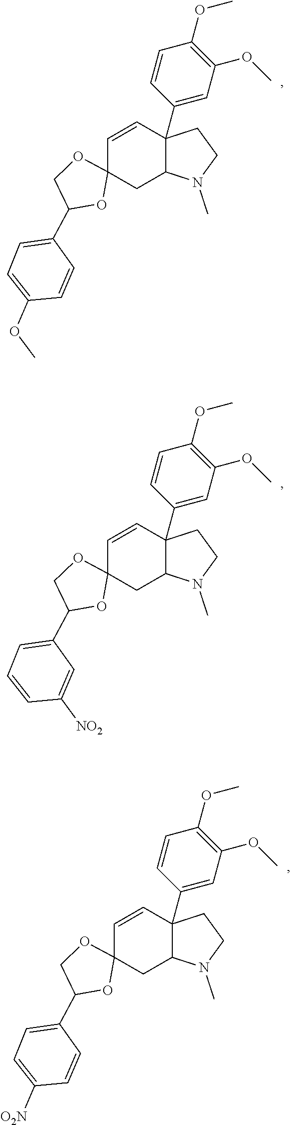 [SMILES: COc1ccc(C2COC3(C=CC4(c5ccc(OC)c(OC)c5)CCN(C)C4C3)O2)cc1 ; COc1ccc(C23C=CC4(CC2N(C)CC3)OCC(c2cccc([N+](=O)[O-])c2)O4)cc1OC ; COc1ccc(C23C=CC4(CC2N(C)CC3)OCC(c2ccc([N+](=O)[O-])cc2)O4)cc1OC]

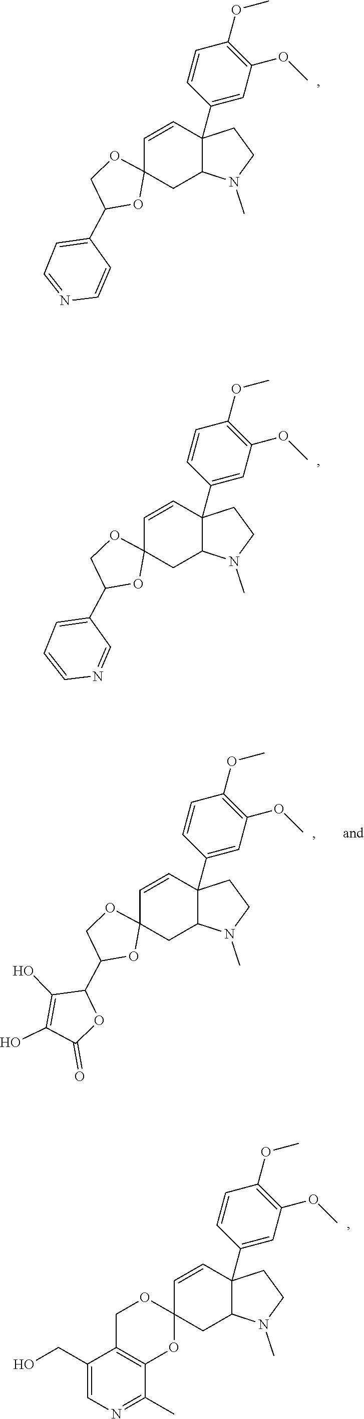 [SMILES: COc1ccc(C23C=CC4(CC2N(C)CC3)OCC(c2ccncc2)O4)cc1OC ; COc1ccc(C23C=CC4(CC2N(C)CC3)OCC(c2cccnc2)O4)cc1OC ; COc1ccc(C23C=CC4(CC2N(C)CC3)OCC(C2OC(=O)C(O)=C2O)O4)cc1OC ; COc1ccc(C23C=CC4(CC2N(C)CC3)OCc2c(CO)cnc(C)c2O4)cc1OC]

or a pharmaceutically acceptable salt thereof.

In some embodiments, the compound is selected from the group consisting of:

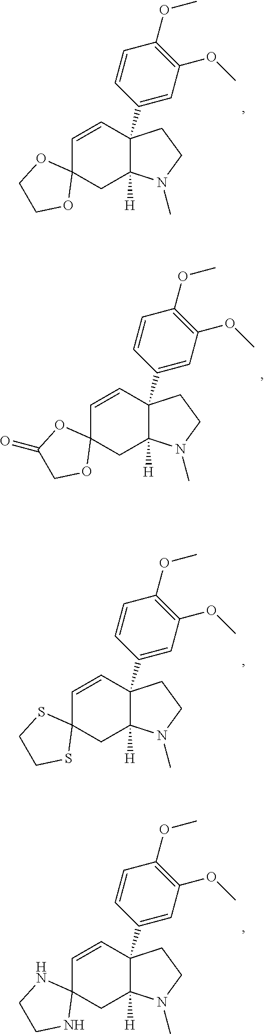 [SMILES: COc1ccc([C@@]23C=CC4(C[C@@H]2N(C)CC3)OCCO4)cc1OC ; COc1ccc([C@@]23C=CC4(C[C@@H]2N(C)CC3)OCC(=O)O4)cc1OC ; COc1ccc([C@@]23C=CC4(C[C@@H]2N(C)CC3)SCCS4)cc1OC ; COc1ccc([C@@]23C=CC4(C[C@@H]2N(C)CC3)NCCN4)cc1OC]

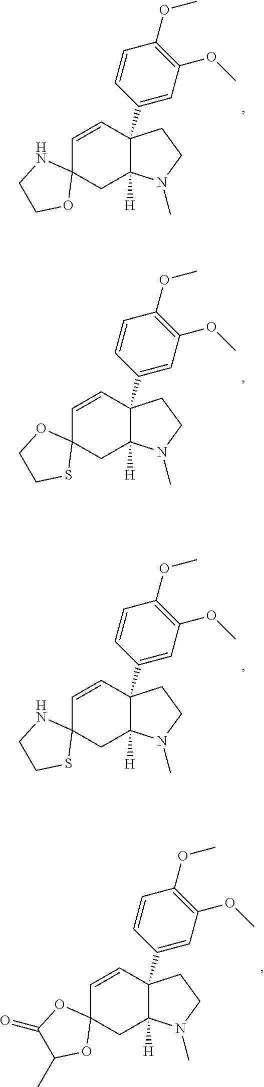 [SMILES: COc1ccc([C@@]23C=CC4(C[C@@H]2N(C)CC3)NCCO4)cc1OC ; COc1ccc([C@@]23C=CC4(C[C@@H]2N(C)CC3)OCCS4)cc1OC ; COc1ccc([C@@]23C=CC4(C[C@@H]2N(C)CC3)NCCS4)cc1OC ; COc1ccc([C@@]23C=CC4(C[C@@H]2N(C)CC3)OC(=O)C(C)O4)cc1OC]

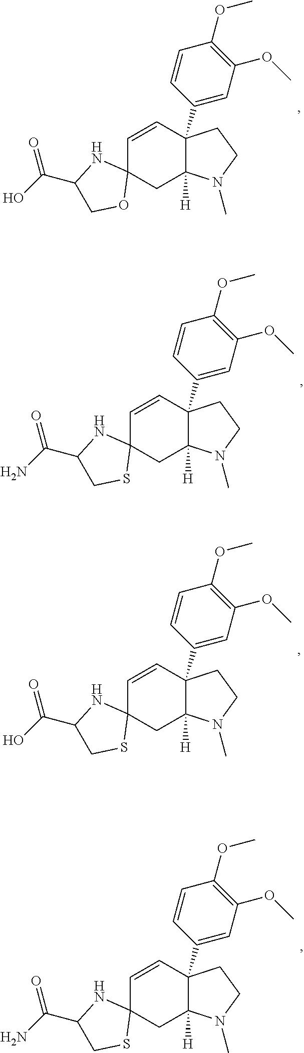 [SMILES: COc1ccc([C@@]23C=CC4(C[C@@H]2N(C)CC3)NC(C(=O)O)CO4)cc1OC ; COc1ccc([C@@]23C=CC4(C[C@@H]2N(C)CC3)NC(C(N)=O)CS4)cc1OC ; COc1ccc([C@@]23C=CC4(C[C@@H]2N(C)CC3)NC(C(=O)O)CS4)cc1OC ; COc1ccc([C@@]23C=CC4(C[C@@H]2N(C)CC3)NC(C(N)=O)CS4)cc1OC]

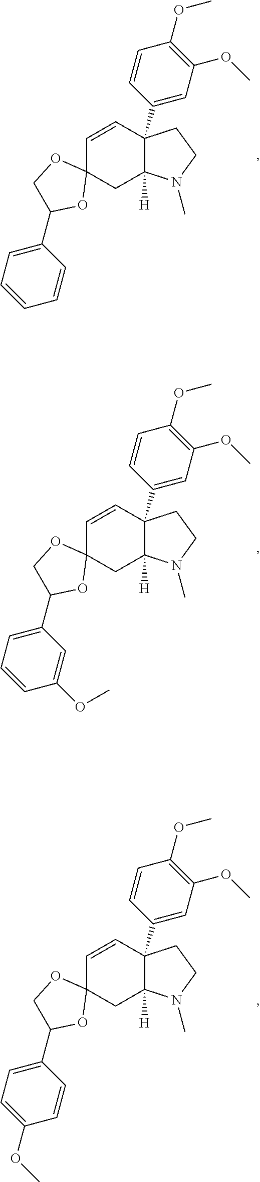 [SMILES: COc1ccc([C@@]23C=CC4(C[C@@H]2N(C)CC3)OCC(c2ccccc2)O4)cc1OC ; COc1cccc(C2COC3(C=C[C@@]4(c5ccc(OC)c(OC)c5)CCN(C)[C@H]4C3)O2)c1 ; COc1ccc(C2COC3(C=C[C@@]4(c5ccc(OC)c(OC)c5)CCN(C)[C@H]4C3)O2)cc1]

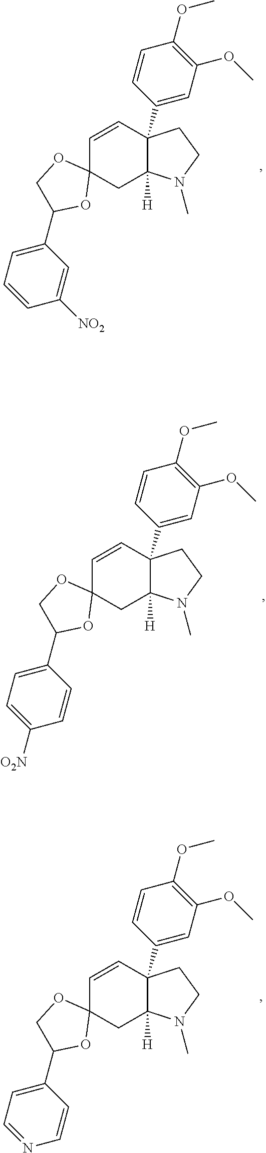 [SMILES: COc1ccc([C@@]23C=CC4(C[C@@H]2N(C)CC3)OCC(c2cccc([N+](=O)[O-])c2)O4)cc1OC ; COc1ccc([C@@]23C=CC4(C[C@@H]2N(C)CC3)OCC(c2ccc([N+](=O)[O-])cc2)O4)cc1OC ; COc1ccc([C@@]23C=CC4(C[C@@H]2N(C)CC3)OCC(c2ccncc2)O4)cc1OC]

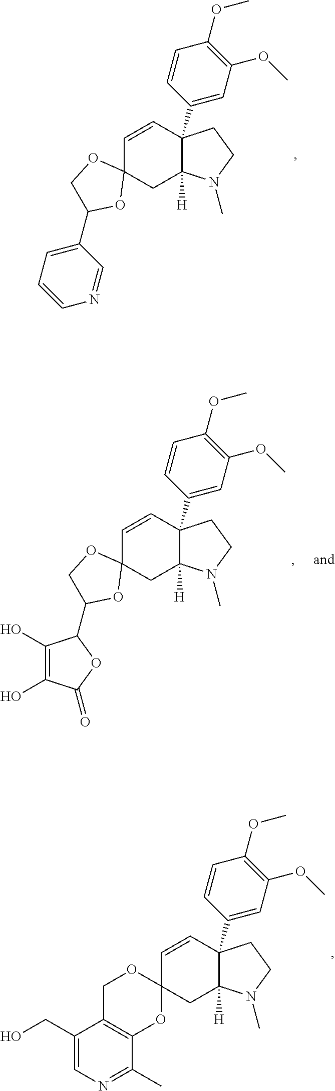 [SMILES: COc1ccc([C@@]23C=CC4(C[C@@H]2N(C)CC3)OCC(c2cccnc2)O4)cc1OC ; COc1ccc([C@@]23C=CC4(C[C@@H]2N(C)CC3)OCC(C2OC(=O)C(O)=C2O)O4)cc1OC ; COc1ccc([C@@]23C=CC4(C[C@@H]2N(C)CC3)OCc2c(CO)cnc(C)c2O4)cc1OC]

or a pharmaceutically acceptable salt thereof.

In some embodiments, the compound is selected from the group consisting of:

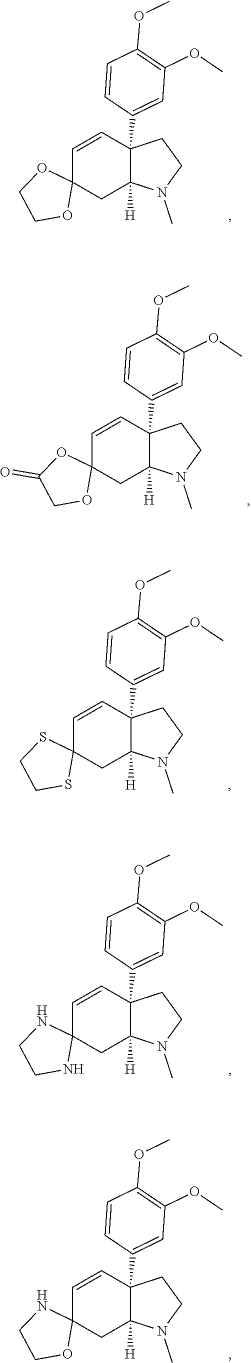 [SMILES: COc1ccc([C@@]23C=CC4(C[C@@H]2N(C)CC3)OCCO4)cc1OC ; COc1ccc([C@@]23C=CC4(C[C@@H]2N(C)CC3)OCC(=O)O4)cc1OC ; COc1ccc([C@@]23C=CC4(C[C@@H]2N(C)CC3)SCCS4)cc1OC ; COc1ccc([C@@]23C=CC4(C[C@@H]2N(C)CC3)NCCN4)cc1OC ; COc1ccc([C@@]23C=CC4(C[C@@H]2N(C)CC3)NCCO4)cc1OC]

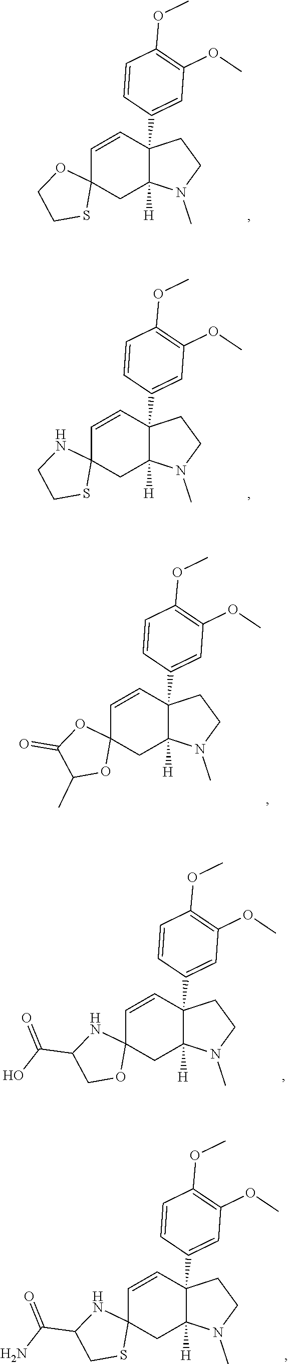 [SMILES: COc1ccc([C@@]23C=CC4(C[C@@H]2N(C)CC3)OCCS4)cc1OC ; COc1ccc([C@@]23C=CC4(C[C@@H]2N(C)CC3)NCCS4)cc1OC ; COc1ccc([C@@]23C=CC4(C[C@@H]2N(C)CC3)OC(=O)C(C)O4)cc1OC ; COc1ccc([C@@]23C=CC4(C[C@@H]2N(C)CC3)NC(C(=O)O)CO4)cc1OC ; COc1ccc([C@@]23C=CC4(C[C@@H]2N(C)CC3)NC(C(N)=O)CS4)cc1OC]

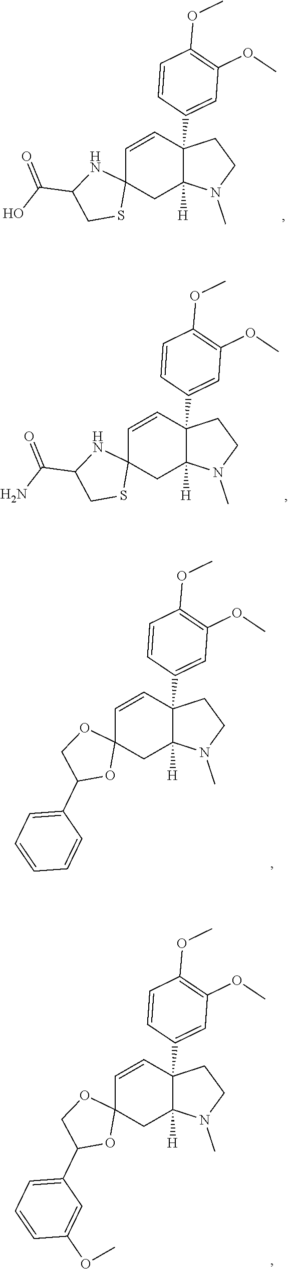 [SMILES: COc1ccc([C@@]23C=CC4(C[C@@H]2N(C)CC3)NC(C(=O)O)CS4)cc1OC ; COc1ccc([C@@]23C=CC4(C[C@@H]2N(C)CC3)NC(C(N)=O)CS4)cc1OC ; COc1ccc([C@@]23C=CC4(C[C@@H]2N(C)CC3)OCC(c2ccccc2)O4)cc1OC ; COc1cccc(C2COC3(C=C[C@@]4(c5ccc(OC)c(OC)c5)CCN(C)[C@H]4C3)O2)c1]

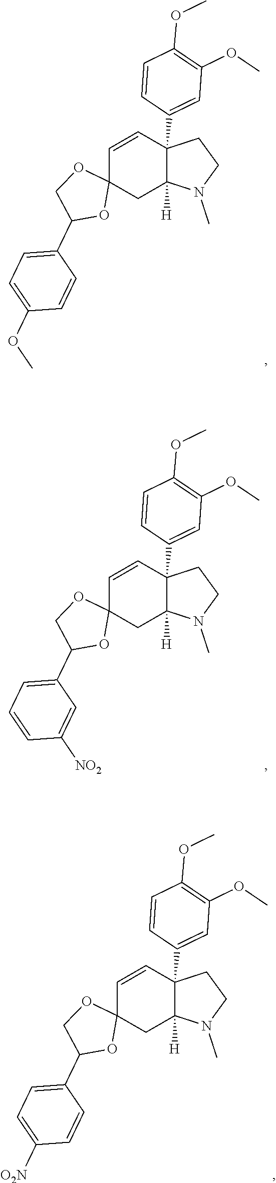 [SMILES: COc1ccc(C2COC3(C=C[C@@]4(c5ccc(OC)c(OC)c5)CCN(C)[C@H]4C3)O2)cc1 ; COc1ccc([C@@]23C=CC4(C[C@@H]2N(C)CC3)OCC(c2cccc([N+](=O)[O-])c2)O4)cc1OC ; COc1ccc([C@@]23C=CC4(C[C@@H]2N(C)CC3)OCC(c2ccc([N+](=O)[O-])cc2)O4)cc1OC]

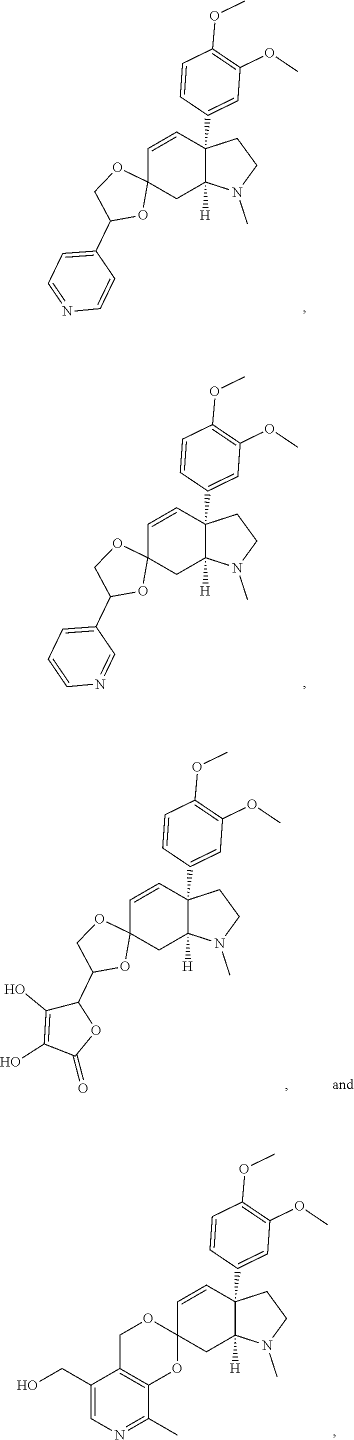 [SMILES: COc1ccc([C@@]23C=CC4(C[C@@H]2N(C)CC3)OCC(c2ccncc2)O4)cc1OC ; COc1ccc([C@@]23C=CC4(C[C@@H]2N(C)CC3)OCC(c2cccnc2)O4)cc1OC ; COc1ccc([C@@]23C=CC4(C[C@@H]2N(C)CC3)OCC(C2OC(=O)C(O)=C2O)O4)cc1OC ; COc1ccc([C@@]23C=CC4(C[C@@H]2N(C)CC3)OCc2c(CO)cnc(C)c2O4)cc1OC]

or a pharmaceutically acceptable salt thereof; and the compound has the absolute stereochemistry shown.

In certain embodiments, compounds described herein convert to mesembrenone as measured by HPLC after 24 hours at a pH of 2 (0.01 M HCl) and a temperature of 37° C. in the Hydrolysis Assay of Example A1.

The present application relates to compounds that can be converted to mesembrenone under conditions encountered within the body, such as upon oral administration. In some embodiments, compounds are provided that hydrolyze to form mesembrenone under acidic conditions (e.g., pH 2) at 37° C. In addition, Applicant has discovered compounds useful for isolating stable forms of individual stereoisomers of mesembrenone.

The invention is based in part on the discovery of compounds having useful and markedly different from naturally occurring mesembrenone, but that can be converted to mesembrenone under biologically relevant conditions. Certain compounds provided herein convert to mesembrenone under physiologically relevant conditions, using the Hydrolysis Assay of Example A1 or A2. In some embodiments, other compounds provided herein can form mesembrenone under biologically relevant acidic conditions. For example, in some embodiments, certain compounds provided herein convert to mesembrenone at acidic pH and temperatures between room temperature and human body temperature (e.g., Compound 15 converted to mesembrenone at acidic pH 2.0 in the Hydrolysis Assay at temperatures of 25° C. as provided in the data in Example A1). In some embodiments, certain compounds provided herein may convert to mesembrenone at acidic pH and temperatures above room temperature including at human body temperature. In contrast, the naturally occurring compound (+)/(−) mesembrenone (herein “Compound 016”) did not further hydrolyze under a variety of biologically relevant conditions ranging from acidic (pH 2.0 in 0.01 M HCl) to neutral buffered conditions (pH 7.4 in 20 mM PBS) from room temperature (25° C.) to elevated temperature (40° C.) (in the Hydrolysis Assay described in Example A1).

In some embodiments, compounds are provided that permit the separation and isolation of stable forms of the (+) mesembrenone separated from the naturally occurring (−) forms of mesembrenone. In certain embodiments, this disclosure provides compositions comprising (+) mesembrenone,

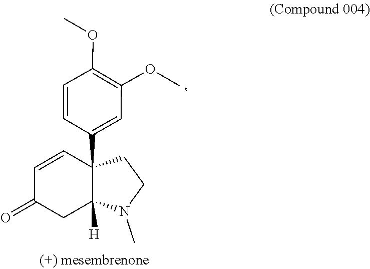 [SMILES: COc1ccc([C@]23C=CC(=O)C[C@H]2N(C)CC3)cc1OC]

or a pharmaceutically acceptable salt thereof.

In some embodiments, (+) mesembrenone compositions can comprise stabilized (+) mesembrenone in the absence of levels of (−) mesembrenone detectable by HPLC. In some embodiments, mesembrenone compositions comprise enriched in (+) mesembrenone compared to (−) mesembrenone. Scheme 1 provides a method of preparing mesembrenone.

The Examples provide non-limiting examples of reactions of racemic and (+) and (−) mesembrenone with various reactive compounds to obtain compounds disclosed herein. In some embodiments, compositions can comprise greater than 15% (w/w) mesembrenone of the total alkaloid content in composition.

In some embodiments, a method of isolating stable forms of (+) mesembrenone and (−) mesembrenone is provided.

In certain embodiments, the present disclosure provides a method of treating a mental disorder, comprising administering to the subject a compound of the present disclosure.

Numerous embodiments are further provided that can be applied to any aspect of the present invention described herein.

### BRIEF DESCRIPTION OF THE DRAWINGS

FIG. 1 is a series of LCMS graphs obtained from compound 015 in the acid hydrolysis assay of Example A1.

FIG. 2 is a series of LCMS graphs obtained from compound 017 in the acid hydrolysis assay of Example A1.

FIG. 3 is a table of SFC separation methods used to separate certain compounds.

FIG. 4 is a table describing purification methods used to separate certain compounds.

FIGS. 5A and 5B are plasma and brain concentrations-time profiles (mean) of 047 and 016 in male C57BL/6 mice following a single oral administration of 047 (Dose: 75 mg/kg) shown in a linear scale (FIG. 5A) and a semi-log scale (FIG. 5B).

### DETAILED DESCRIPTION

The present invention is based, at least in part, on mesembrenone and analogs thereof. Although mesembrenone is bioactive with certain desirable pharmacologic effects, certain other properties are less than ideal for use as a therapeutic. To take advantage of the desirable properties of mesembrenone and improve upon absorption, distribution, metabolism and excretion (ADME) that impact pharmacokinetics (PK), compounds have been developed and described here. At least some of the compounds have the shared properties characterized by one or more of the following: (1) they have a function group manipulation at, or related to, the ketone; (2) the modification to the structure impacts physiochemical properties; (3) they break down in the presence of aqueous acid to form mesembrenone (e.g., (−) mesembrenone); (4) they are intended to tune the ADME/PK of mesembrenone (e.g., (−) mesembrenone) in vivo. An in vitro aqueous hydrolysis assay to that may predict parameters associated with absorption and pharmacokinetics in vivo is also described here.

### Exemplary Compounds of the Invention

In certain embodiments, compounds described herein can form mesembrenone (e.g., (−) mesembrenone) under biologically relevant conditions. For example, in some embodiments, compounds of disclosed herein (e.g., compounds of Formula (I)) can hydrolyze in highly acidic environments (e.g., pH of about 2 at room temperature or more comparably stringent conditions typically encountered within the alimentary canal of a mammal) at a rate that is advantageous for providing a desired bioabsorption (% F) following oral administration by a mammal and leading to a desired pharmacokinetic profile of mesembrenone (e.g., (−) mesembrenone) to the mammal.

In some embodiments, a compound according to the present disclosure is of formula (I):

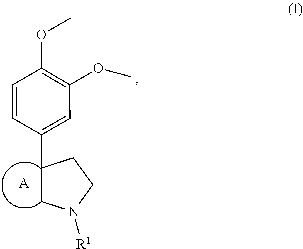

or a pharmaceutically acceptable salt thereof, wherein
R¹ is H or C₁-C₇ alkyl and
ring A is

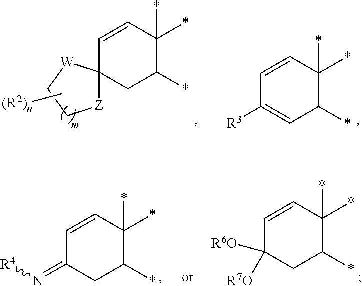

wherein * denotes the attachment points of ring A to the compound of formula (I), and wherein
each of W and Z is independently O, NH, or S;
each R² is independently C₁-C₃ alkyl, C₂-C₆ alkenyl, —COOH, —CONH₂, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol;
m is 1 or 2;
n is 0, 1, 2, or 3;
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl; and
each of R⁶ and R⁷ is independently C₁-C₃ alkyl.

In some embodiments, a compound according to the present disclosure is of formula (I):

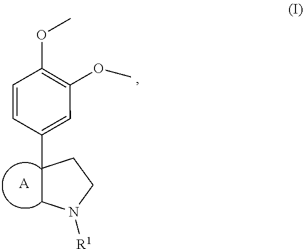

or a pharmaceutically acceptable salt thereof, wherein
R¹ is H or C₁-C₇ alkyl; and
ring A is

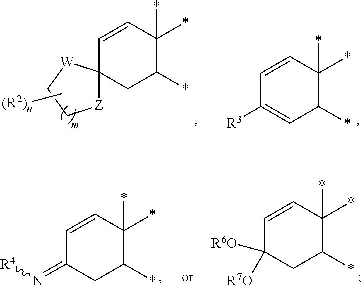

wherein * denotes the attachment points of ring A to the compound of formula (I), and wherein
each of W and Z is independently O, NH, or S;
each R² is independently C₁-C₃ alkyl, C₂-C₆ alkenyl, —COOH, —CONH₂, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol;
m is 1 or 2;
n is 0, 1, or 2;
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl; and
each of R⁶ and R⁷ is independently C₁-C₃ alkyl.

In some embodiments, a compound according to the present disclosure is of formula (I):

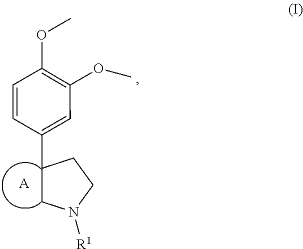

or a pharmaceutically acceptable salt thereof.

In some embodiments ring A is

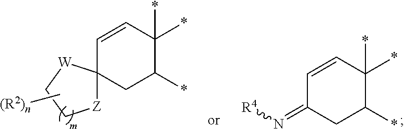

wherein* denotes the attachment points of ring A to the compound of formula (I).

In some embodiments, each of W and Z is independently O, NH, or S. In some embodiments, W is O. In some embodiments, W is NH. In some embodiments, W is S.

In some embodiments, each R² is independently C₁-C₃ alkyl, C₂-C₆ alkenyl, —COOH, —CONH₂, phenyl, 5- to 7-membered heterocyclyl, or 5- to 7-membered heteroaryl. In some embodiments, each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, C₁-C₃ alkanol, or —NO₂. In some embodiments, R² is —COOH, —CONH₂, phenyl, 5- to 7-membered heterocyclyl, or 5- to 7-membered heteroaryl, wherein each hydrogen atom in phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl. In some embodiments, R² is phenyl, wherein each hydrogen atom in phenyl is optionally substituted by —OH, C₁-C₃ alkyl, C₁-C₃ alkoxy, or —NO₂. In some embodiments, R² is 5- to 7-membered heterocyclyl or 5- to 7-membered heteroaryl, wherein each hydrogen atom in 5- to 7-membered heterocyclyl and 5- to 7-membered heteroaryl is optionally substituted by —OH, C₁-C₃ alkyl, C₁-C₃ alkoxy, C₁-C₃ alkanol, or —NO₂. In some embodiments, R² is pyridyl. In some embodiments, R² is

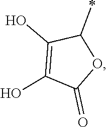 [SMILES: *C1OC(=O)C(O)=C1O]

wherein * denotes the point of attachment of R² the compound. In some embodiments, R² is —COOH or —CONH₂.

In certain embodiments, the compound is of formula (I-1):

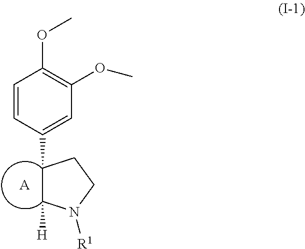

or a pharmaceutically acceptable salt thereof, wherein
R¹ is H or C₁-C₇ alkyl; and
ring A is

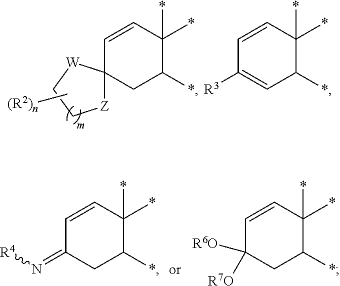

wherein * denotes the attachment points of ring A to the compound of formula (I), and wherein
each of W and Z is independently O, NH, or S;
each R² is independently C₁-C₃ alkyl, C₂-C₆ alkenyl, —COOH, —CONH₂, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol;
m is 1 or 2;
n is 0, 1, 2, or 3;
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl; and
   each of R⁶ and R⁷ is independently C₁-C₃ alkyl.

In certain embodiments, the compound is of formula (I-1):

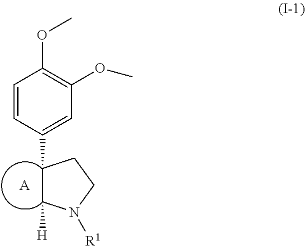

or a pharmaceutically acceptable salt thereof, wherein
   R¹ is H or C₁-C₇ alkyl; and
   ring A is

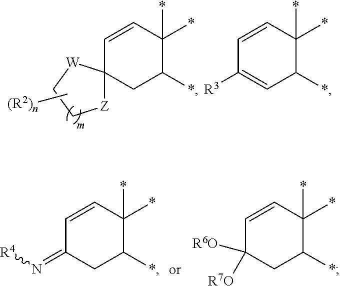

wherein * denotes the attachment points of ring A to the compound of formula (I), and wherein
each of W and Z is independently O, NH, or S;
each R² is independently C₁-C₃ alkyl, C₂-C₆ alkenyl, —COOH, —CONH₂, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol;
m is 1 or 2;
n is 0, 1, or 2;
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl; and
each of R⁶ and R⁷ is independently C₁-C₃ alkyl.

In certain embodiments, the compound is of formula (I-1):

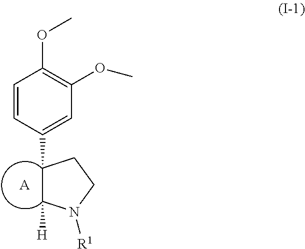

or a pharmaceutically acceptable salt thereof, wherein
R¹ is H or C₁-C₇ alkyl; and
ring A is

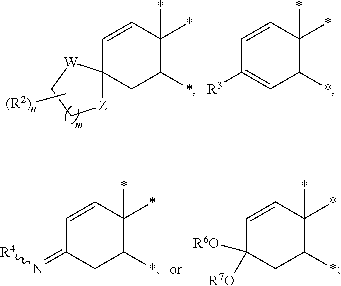

wherein * denotes the attachment points of ring A to the compound of formula (I), and wherein
each of W and Z is independently O, NH, or S;
each R² is independently C₁-C₃ alkyl, C₂-C₆ alkenyl, —COOH, —CONH₂, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol;
m is 1 or 2;
n is 0, 1, 2, or 3;
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl;
each of R⁶ and R⁷ is independently C₁-C₃ alkyl; and
the compound of formula (I-1) has the absolute stereochemistry shown.

In certain embodiments, the compound is of formula (I-1):

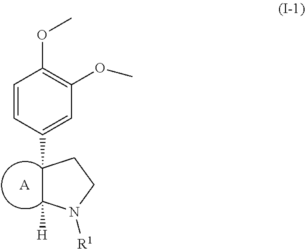

or a pharmaceutically acceptable salt thereof, wherein
R¹ is H or C₁-C₇ alkyl; and
ring A is

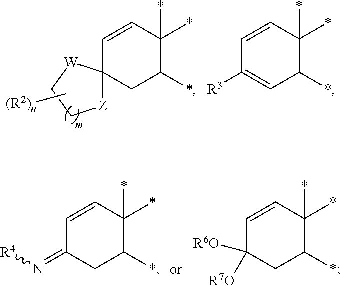

wherein * denotes the attachment points of ring A to the compound of formula (I), and wherein
each of W and Z is independently O, NH, or S;
each R² is independently C₁-C₃ alkyl, C₂-C₆ alkenyl, —COOH, —CONH₂, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol;
m is 1 or 2;
n is 0, 1, or 2;
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl;
each of R⁶ and R⁷ is independently C₁-C₃ alkyl; and
the compound of formula (I-1) has the absolute stereochemistry shown.

In some embodiments, two R²s on a single carbon atom combine to form ═O.

In some embodiments, two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol.

In some embodiments, the two R²s together with the carbon atoms to which they are attached combine to form pyridyl, wherein each hydrogen atom in pyridyl is optionally substituted by —OH, C₁-C₃ alkyl, or C₁-C₃ alkanol.

In some embodiments, m is 1 or 2.

In some n is 0, 1, or 2. In some embodiments, n is 0. In some embodiments, n is 1. In some embodiments, n is 2.

In some n is 0, 1, 2, or 3. In some embodiments, n is 0. In some embodiments, n is 1. In some embodiments, n is 2. In some embodiments, n is 3. In some embodiments, n is 4.

In some embodiments, R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5-to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy. In some embodiments, of R³ is —OC(O)C₁-C₆ alkyl and the C₁-C₆ alkyl is methyl, ethyl, butyl, isobutyl, tertbutyl, or 2-methylbutyl, wherein each hydrogen atom in methyl, ethyl, butyl, isobutyl, tertbutyl, and 2-methylbutyl is optionally substituted by halogen or phenyl. In some embodiments, R³ is —OC(O)C₂-C₆ alkenyl (e.g., isopropenyl or butenyl). In some embodiments, is —OC(O)C₃-C₁₀ cycloalkyl (e.g., cyclopropyl or cyclohexyl). In some embodiments, R³ is —OC(O)phenyl, wherein each hydrogen atom in phenyl is optionally substituted by methyl, isopropyl, tertbutyl, chloro, fluoro, CF₃, —CHF₂, cyano, —N(CH₃)₂, -methoxy, or nitro. In some embodiments, —OC(O)-5- to 7-membered heteroaryl, which may be optionally substituted (e.g., pyridyl or thiophenyl, wherein each hydrogen atom in pyridyl or thiophenyl is optionally substituted by methyl).

In some embodiments, R⁴ is OH, C₁-C₆ alkoxy, or —NHC(O)-5- to 7-membered heteroaryl. In some embodiments, each hydrogen atom in 5- to 7-membered heteroaryl is optionally substituted by —OH, C₁-C₃ alkyl, or C₁-C₃ alkanol.

In some embodiments, R⁴ is —OR⁵ or —N(R⁵)₂.

In some embodiments, each R⁵ is H, C₁-C₆ alkyl, C₃-C₆ heterocycloalkyl, C₁-C₆ alkoxy, phenyl, —C(O)-5- to 7-membered heteroaryl, —C(O)—C₁-C₃ alkyl, —C(O)-phenyl, -C(O)N(H)-phenyl, or —CONH₂. In some embodiments, each hydrogen atom in C₁-C₆ alkyl, C₁-C₆ alkoxy phenyl, and 5- to 7-membered heteroaryl is optionally substituted by halogen, OH, C₁-C₃ alkyl, C₃-C₆ cycloalkyl, phenyl, 5- to 7-membered heteroaryl aryloxy (e.g., phenoxy), C₁-C₆ alkoxy, C₁-C₃ alkanol, —COOH, —C(O)C₁-C₆ alkyl, or —C(O)OC₁-C₆ alkyl.

In some embodiments, R¹ is H or C₁-C₇ alkyl. In some embodiments, R¹ is C₁-C₃ alkyl. In some embodiments, R¹ is methyl.

In some embodiments, the compound is of formula (V):

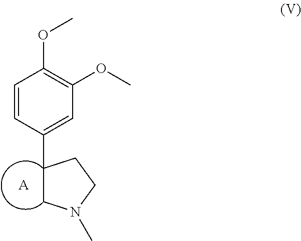

or a pharmaceutically acceptable salt thereof,
wherein
ring A is

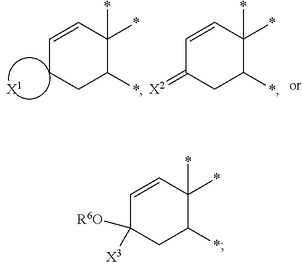

wherein * denotes the attachment points of ring A to the compound of formula (I), and wherein
R⁶ is C₁-C₃ alkyl;
X¹ is >L-R¹⁴, wherein L is absent or a linker and where > denotes the two single bonds to the cyclohexane ring such that the ring containing X¹ forms a C₃-C₆ alkyl ring, which is optionally substituted by C₁-C₃ alkanol;
X² is =L-R¹⁴, wherein L is absent or a linker;
X³ is -L-R¹⁴, wherein L is absent or a linker; and
R¹⁴ comprises a generally recognized as safe (GRAS) compound.

In some embodiments, the compound is of formula (V-1):

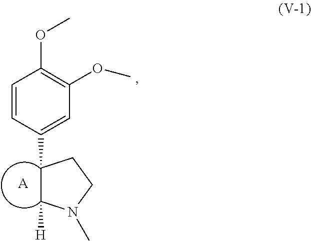

or a pharmaceutically acceptable salt thereof,
wherein
ring A is

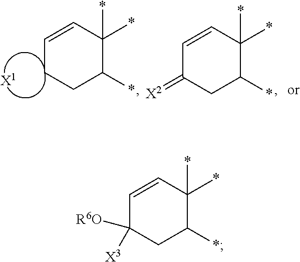

wherein * denotes the attachment points of ring A to the compound of formula (I), and wherein
R⁶ is C₁-C₃ alkyl;
X¹ is >L-R¹⁴, wherein L is absent or a linker and where > denotes the two single bonds to the cyclohexane ring such that the ring containing X¹ forms a C₃-C₆ alkyl ring, which is optionally substituted by C₁-C₃ alkanol;
X² is =L-R¹⁴, wherein L is absent or a linker;
X³ is -L-R¹⁴, wherein L is absent or a linker;
R¹⁴ comprises a generally recognized as safe (GRAS) compound; and
the compound of formula (V-1) has the absolute stereochemistry shown.

In some embodiments, -L-R¹⁴ comprises a GRAS moiety. In some embodiments, is -L-R¹⁴ is selected from

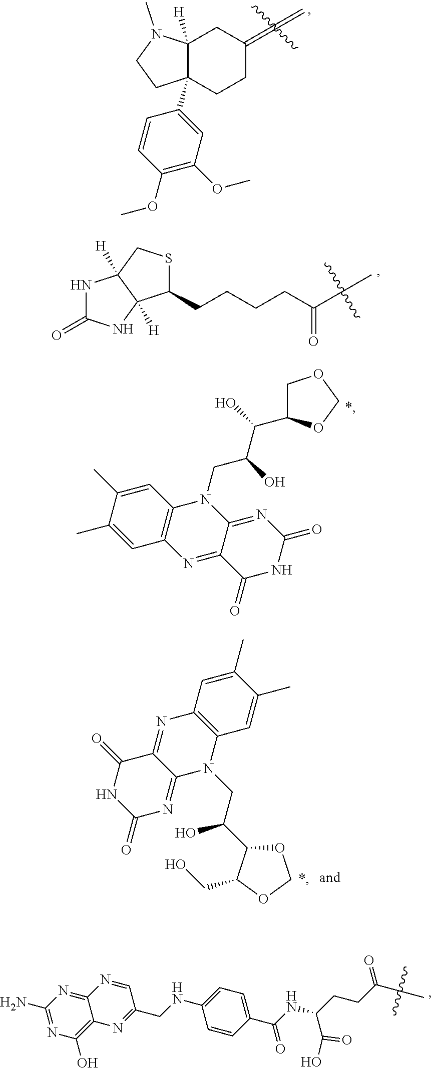

wherein the * denotes the carbon of the cyclohexyl ring when L is absent.

In some embodiments, the compounds of Formula (I) can be obtained by reacting mesembrenone (M) with a compound designated as Generally Recognized as Safe (GRAS) by the U.S. Food and Drug Administration (FDA) (G) under sections 201(s) and 409 of the U.S. federal Food, Drug and Cosmetics Act (FDCA), and under the corresponding implementing regulations in 21 CFR 170.3 and 21 CFR 170.30. For example, compounds that convert to mesembrenone can be obtained by reaction of either compound with lactic acid, glycolic acid, ascorbic acid (vitamin C), and pyridoxine (vitamin B6) derivatives. Table 2 provides examples of compounds that can be reacted to obtain compounds of Formula (I) by the reaction: Compound M+Compound R→Compound C.

TABLE 2

| Reactant M | Reactant R | Product Compound C |
|---|---|---|
| 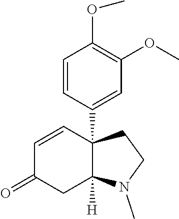 [SMILES: COc1ccc([C@@]23C=CC(=O)C[C@@H]2N(C)CC3)cc1OC] | 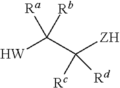 | 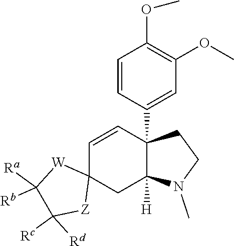 |
| 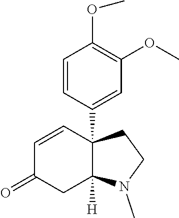 [SMILES: COc1ccc([C@@]23C=CC(=O)C[C@@H]2N(C)CC3)cc1OC] | 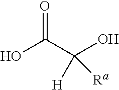 | 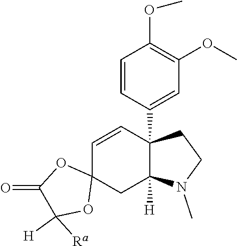 |
| 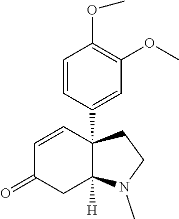 [SMILES: COc1ccc([C@@]23C=CC(=O)C[C@@H]2N(C)CC3)cc1OC] | 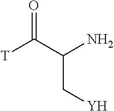 | 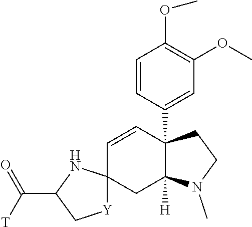 |
| 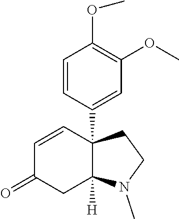 [SMILES: COc1ccc([C@@]23C=CC(=O)C[C@@H]2N(C)CC3)cc1OC] | 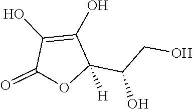 [SMILES: O=C1O[C@H]([C@@H](O)CO)C(O)=C1O] | 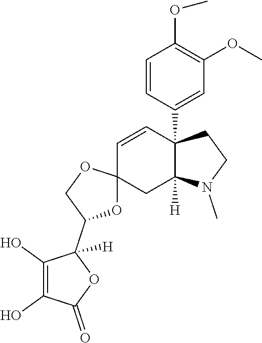 |
| 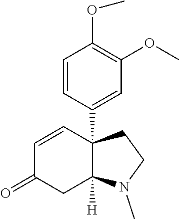 [SMILES: COc1ccc([C@@]23C=CC(=O)C[C@@H]2N(C)CC3)cc1OC] | 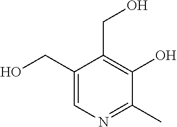 | 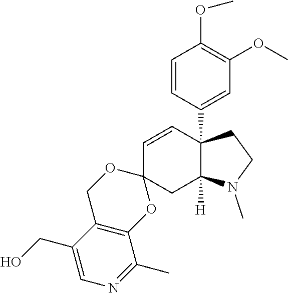 [SMILES: COc1ccc([C@@]23C=CC4(C[C@@H]2N(C)CC3)OCc2c(CO)cnc(C)c2O4)cc1OC] |
| 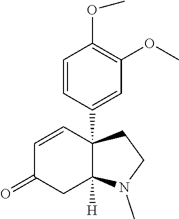 [SMILES: COc1ccc([C@@]23C=CC(=O)C[C@@H]2N(C)CC3)cc1OC] | 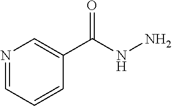 | 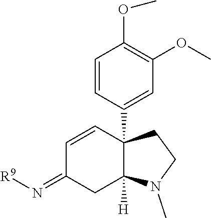 |

Unless otherwise indicated in the tables of compounds herein, the abbreviation RAC or rac indicates a racemic mixture, and DIAST indicates a specific diastereomer. In illustrative embodiments, although a compound may be depicted with [symbol] or [symbol] bonds, such a depiction may be denoting relative stereochemistry based on elution peaks from a chiral separation.

Compounds of Formula (I) including compounds of Formula (IIa) and Formula (IIb) can be prepared as described with respect to exemplary compounds in the examples below. In general, treatment of a ketone, such as mesembrenone, with an appropriate diol or dithiane, and an acid catalyst, such as p-toluenesulfonic acid or methanesulfonic acid, in a solvent, such as toluene, at an elevated temperature is a method to prepare compounds of Formula (IIa-IIb).

TABLE 1
Exemplary Compounds of Formula IIa-IIb

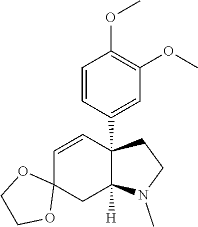 [SMILES: COc1ccc([C@@]23C=CC4(C[C@@H]2N(C)CC3)OCCO4)cc1OC]

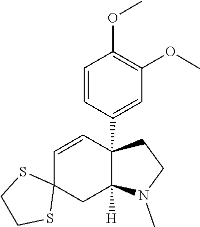 [SMILES: COc1ccc([C@@]23C=CC4(C[C@@H]2N(C)CC3)SCCS4)cc1OC]

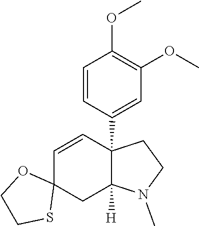 [SMILES: COc1ccc([C@@]23C=CC4(C[C@@H]2N(C)CC3)OCCS4)cc1OC]

In some embodiments, the compound of formula (I) is a compound of formula (IX):

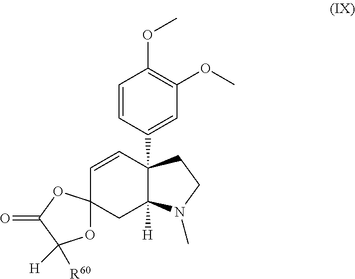

or a pharmaceutically acceptable salt thereof, wherein the dashed bond is absent or present, and R₆₀ is hydrogen or methyl.

In some embodiments, a compound of Formula (IX) c converts to mesembrenone (e.g., (−) mesembrenone) upon oral administration to a mammal and upon hydrolysis in sufficiently acidic conditions (e.g., pH 2 at 37 degrees C.), where R₆₀ is as defined with respect to Formula (I-B) above.

Compounds of Formula (IX) can be prepared as described with respect to compounds in the examples below. In general treatment of a ketone, such as mesembrenone, with an appropriate alpha-hydroxy carboxylic acid, with a Lewis acid, such as boron trifluoride etherate, in a solvent, such as dichloromethane, is a method to prepare compounds of Formula (IX). For example, a compound of formula (IX) where R₆₀ is methyl can be obtained by reacting mesembrenone with lactic acid (e.g., with TsOH, toluene). In some embodiments, the compound can be a lactate or glycolate derivative of mesembrenone. R₆₀ in Scheme 2 is as defined above with respect to Formula (I-B).

Table 2 provides non-limiting examples of certain compounds of Formula (IX).

TABLE 2
Exemplary Compounds of Formula IX

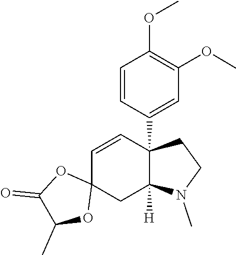

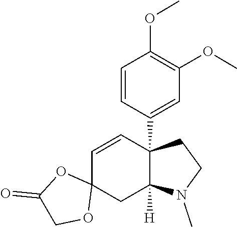 [SMILES: COc1ccc([C@@]23C=CC4(C[C@@H]2N(C)CC3)OCC(=O)O4)cc1OC]

In some embodiments, the compound of Formula (I) is a compound of Formula (X) or a pharmaceutically acceptable salt thereof:

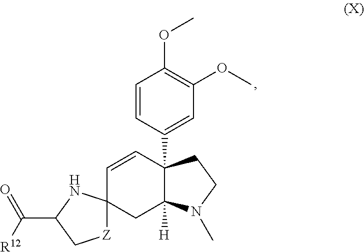

wherein Z is S or O; and R₁₂ is hydroxyl or amino.

In some embodiments, a compound of Formula (X) can convert to mesembrenone (e.g., (−) mesembrenone) upon oral administration to a mammal and upon hydrolysis in sufficiently acidic conditions (e.g., pH 2 at 37° C.), where Z and R₁₂ are as defined with respect to Formula (X) above.

Compounds of Formula (X) can be prepared as described with respect to exemplary compounds in the examples below. In general, treatment of a ketone, such as mesembrenone, with an appropriate alpha-amino carboxylic acid or alpha-amino amide, in a solvent, such as ethanol, at an elevated temperature is a method to prepare compounds of Formula (X).

In some embodiments, the compound of Formula (I) is a compound of Formula (XI) or a pharmaceutically acceptable salt thereof:

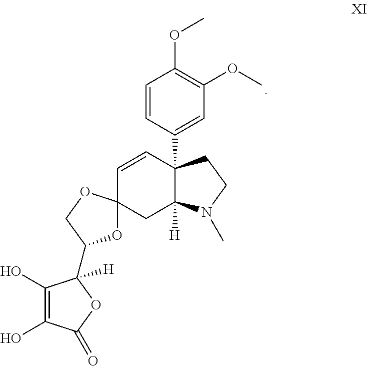

In some embodiments, a compound of Formula (XI) can convert to mesembrenone (e.g., (−) mesembrenone) upon oral administration to a mammal and upon hydrolysis in sufficiently acidic conditions (e.g., pH 2 at 37° C.). In some embodiments, the compound can be an ascorbate derivative of mesembrenone.

Compounds of Formula (XI) can be prepared as described with respect to compounds in the examples below. In general, treatment of a ketone such as mesembrenone, with a naturally occurring diol, such as ascorbic acid, with an acid catalyst in a solvent, such as toluene, is a method to prepare compounds of Formula (XI). In some embodiments, mesembrenone can be reacted with ascorbic acid (e.g., TsOH, acetone) to obtain a compound of Formula (XI-2).

Table 4 provides non-limiting examples of certain compounds of Formula (XI). Table 4. Exemplary Compounds of Formula XI

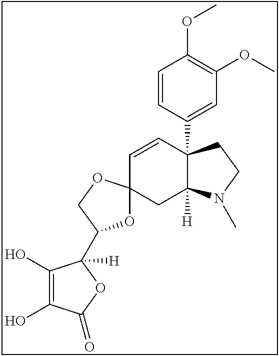

In some embodiments, the compound of Formula (I) is a compound of Formula (XII) or a pharmaceutically acceptable salt thereof:

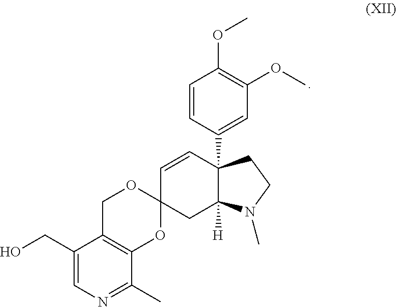 [SMILES: COc1ccc([C@@]23C=CC4(C[C@@H]2N(C)CC3)OCc2c(CO)cnc(C)c2O4)cc1OC]

In some embodiments, a compound of Formula (XII) can convert to mesembrenone (e.g., (−) mesembrenone) upon oral administration to a mammal and upon hydrolysis in sufficiently acidic conditions (e.g., pH 2 at 37° C.).

Compounds of Formula (XII)) can be prepared as described with respect to exemplary compounds in the examples below. In general treatment of a ketone such as mesembrenone, with a compound such as pyridoxine or an analog thereof (e.g., a vitamin B6 analog), with an acid catalyst in a solvent is a method to prepare compounds of Formula (XII).

Table 5 provides non-limiting examples of certain compounds of Formula (XII). Table 5. Exemplary Compounds of Formula XII

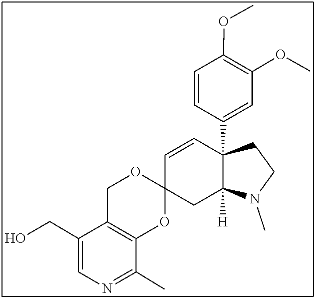 [SMILES: COc1ccc([C@@]23C=CC4(C[C@@H]2N(C)CC3)OCc2c(CO)cnc(C)c2O4)cc1OC]

In some embodiments, the compound is a compound of Formula (XV) or a pharmaceutically acceptable salt thereof:

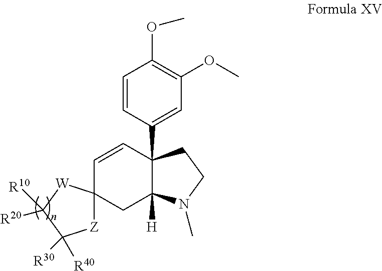

wherein
n is 0 or 1,
W and Z are each independently CH₂, O, S or NH, provided that at least one of W or Z is O, S or NH; and
R₁₀, R₂₀, R₃₀ and R₄₀ are each hydrogen.

In some embodiments, W and Z in Formula (XV) are each O. In some embodiments, W and Z in Formula (XV) are each S. In some embodiments, W and Z in Formula (XV) are each NH. In some embodiments, W is NH and Z is S in Formula (XV). In some embodiments, W is O and Z is S in Formula (XV). In some embodiments, W is CH₂ and Z is O in Formula (XV). In some embodiments, W is CH₂ and Z is NH in Formula (XV).

Compounds of Formula (XV) can be prepared as described with respect to exemplary compounds in the examples below.

In some embodiments, compositions can comprise greater than 15% (w/w) mesembrenone of the total alkaloid content in composition. In some embodiments, compositions can comprise greater than 50% (w/w) mesembrenone of the total alkaloid content in composition. In some embodiments, compositions can comprise greater than 90% (w/w) mesembrenone of the total alkaloid content in composition. In some embodiments, compositions can comprise greater than 99% (w/w) mesembrenone of the total alkaloid content in composition.

In some embodiments, compositions can comprise greater than 15% (w/w) of mesembrenone (e.g., (−) mesembrenone) of the total alkaloid content in composition. In some embodiments, compositions can comprise greater than 50% (w/w) mesembrenone (e.g., (−) mesembrenone) of the total alkaloid content in composition. In some embodiments, compositions can comprise greater than 90% (w/w) mesembrenone (e.g., (−) mesembrenone) of the total alkaloid content in composition. In some embodiments, compositions can comprise greater than 99% (w/w) mesembrenone (e.g., (−) mesembrenone) of the total alkaloid content in composition.

In some embodiments, many prodrugs of mesembrenone (e.g., (−) mesembrenone are disclosed herein. Illustratively, converting mesembrenone to a prodrug can be performed by modifying the ketone on the fused ring. In some embodiments, the modification can be take the form of a protecting group. Illustrative ketone protecting groups are known in the art as described in Greene's Protective Groups in Organic Synthesis, fourth edition, the disclosure of which is hereby incorporated by reference. Illustrative ketone protecting groups include acyclic ketals, cyclic ketals, chiral ketals, dithio ketals, cyclic dithio ketals, monothiol ketals, cyclic monothiol ketals, cyanohydrins, hydrazones, oximes, pyrrole carbinols, O-silylimdazoyl aminals, cyclic aminals, and benzothiazoles. Ketones may also be protected by protecting the enolate, for example by trimethylsilyl enol ethers, and enamines,

In some embodiments, a method of isolating stable forms of (+) mesembrenone and (−) mesembrenone is provided. In illustrative embodiments, the method minimizes racemization of mesembrenone. In some embodiments, stereoisomer analogs of (+) mesembrenone and (−) mesembrenone can be formed. The (+)/(−) mesembrenone analogs can then be separated. The (+) analog can then be converted to (+) mesembrenone. The (−) analog can then be converted to (−) mesembrenone. The conversion can be performed by hydrolysis, preferably in the presence of an acid.

In illustrative embodiments, a method of extending the pharmacokinetic properties of mesembrenone is described. In illustrative embodiments, the pharmacokinetic properties of mesembrenone is extended by forming a prodrug, for example by modifying the ketone on the fused ring.

In some embodiments, compounds of Formula (I-1) can form mesembrenone (e.g., (−) mesembrenone) under biologically relevant conditions, including compounds of formula (I-1), formula (IIb-1), formula (IIIb). In certain embodiments, methods of administering a therapeutic alkaloid compound comprise the oral administration of a compound of formula (I-1), formula (IIb-1), and formula (IVb-1). In certain embodiments, methods of administering a therapeutically effective amount of mesembrenone can comprise the step of administering a compound of formula (IIb-1).

In some embodiments, methods of treating a patient suffering from a disease comprise administering to a patient a composition comprising a compound disclosed herein for the treatment or prevention of a mental health disorder. In some embodiments, methods of treating a patient suffering from a disease comprise administering to a patient a composition comprising a compound disclosed herein for the treatment or prevention of a diagnosed condition selected from anxiety and depression. In some embodiments, a method comprises the administration to a patient in need thereof of a therapeutically effective amount of a compound of Formula (I) for the treatment of depression. In some embodiments, a method comprises the administration to a patient in need thereof of a therapeutically effective amount of a compound of Formula (I) for the treatment of a condition selected from the group consisting of: anxiety associated with depression, anxiety with depression, mixed anxiety and depressive disorder. In some embodiments, a method comprises the administration to a patient in need thereof of a therapeutically effective amount of a compound of Formula (I) for the treatment of anxiety and hysteria or anxiety and depression.

In some embodiments, the compound disclosed herein is administered to the patient in a unit dose. In some embodiments, the compound disclosed herein is prescribed to a patient in an oral unit dose for such as a capsule or tablet once or more times per day. In some embodiments, a compound disclosed herein is administered to a patient for the treatment of a disease or condition for which mesembrenone is safe and effective for treatment. In some embodiments, a method comprises the administration to a patient in need thereof of a therapeutically effective amount of a compound of Formula (I) for the treatment of anxiety. In some embodiments, a method comprises the administration to a patient in need thereof of a therapeutically effective amount of a compound of Formula (I) for the treatment of a disease selected from the group consisting of mild to moderate depression and major depressive episodes. In some embodiments, a method comprises the administration to a patient in need thereof of a therapeutically effective amount of a compound of Formula (I) for the treatment of a disease selected from the group consisting of psychological and psychiatric disorders where anxiety is present. In some embodiments, a method comprises the administration to a patient in need thereof of a therapeutically effective amount of a compound of Formula (I) for the treatment of major depressive episodes. In some embodiments, a method comprises the administration to a patient in need thereof of a therapeutically effective amount of a compound of Formula (I) for the treatment of a disease selected from the group consisting of alcohol and drug dependence, bulimia nervosa, and obsessive-compulsive disorders. In some embodiments, an amount of from 20 micrograms to 2 milligrams of a compound of Formula (I) is orally administered to a patient to treat the patient in need thereof with a therapeutic compound selected from the group consisting of (−)/(+) mesembrenone, (−) mesembrenone and (+) mesembrenone. In some embodiments, an amount of from 20 micrograms to 2 milligrams of a compound of Formula (I) is orally administered to a patient to treat the patient in need thereof with a therapeutic compound selected from the group consisting of (−)/(+) mesembrenone.

### Pharmaceutical Compositions

In certain embodiments, the present application is directed to a pharmaceutical composition comprising an active pharmaceutical ingredient. In certain embodiments, the pharmaceutical composition comprises a compound as disclosed herein as the active pharmaceutical ingredient (API) and a pharmaceutically acceptable carrier comprising one or more excipients. In some embodiments, the pharmaceutical composition optionally further comprises an additional therapeutic compound (i.e., agent) with the pharmaceutically acceptable carrier. The pharmaceutical composition can be a medicament.

Pharmaceutically acceptable carriers include those known in the art. The choice of a pharmaceutically acceptable carrier can depend, for example, on the desired route of administration of the composition. A pharmaceutical composition (preparation) can be administered to a subject by any of a number of routes of administration including, for example, parenteral administration (e.g. intravenously, subcutaneously, or intramuscularly), oral administration (for example, tablets, and capsules); absorption through the oral mucosa (e.g., sublingually) or transdermally (for example as a patch applied to the skin) or topically (for example, as a cream, ointment or spray applied to the skin).

In some embodiments, pharmaceutical compositions comprising compounds of Formula (I) or pharmaceutically acceptable salts thereof can be formulated for oral administration. For example, a compound provided herein can be combined with suitable compendial excipients to form an oral unit dosage form, such as a capsule or tablet, containing a target dose of a compound of Formula (I). The drug product can be prepared by first manufacturing the compound of Formula (I) as an active pharmaceutical ingredient (API), followed by roller compaction/milling with intragranular excipients and blending with extra granular excipients. A Drug Product can contain the selected compound of Formula (I) as the API and excipient components in a tablet in a desired dosage strength of a compound of Formula (I). The blended material can be compressed to form tablets and then film coated. The excipients can be selected from materials appropriate for inclusion in a pharmaceutical composition for an intended purpose and route of delivery including providing a desired manufacturing and stability properties and/or desired in vivo characteristics or other properties to the pharmaceutical composition. In some embodiments, the pharmaceutical composition can include a compound of Formula (I) as the API in combination with a filler (e.g., a form of microcrystalline cellulose), a dry binder or disintegrant (e.g., a cross-linked polymer), a glidant (e.g., colloidal silicon dioxide) and/or a lubricant (e.g., magnesium stearate). In some embodiments, the pharmaceutical composition can comprise a material such as an extended release or disintegrant involved in carrying or transporting the API pharmaceutical agent from one organ, or portion of the body, to another organ, or portion of the body of a subject, including materials to desirable control the absorption of the API in the intestine.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

To prepare solid dosage forms for oral administration, the active ingredient is mixed with one or more pharmaceutically acceptable carriers, such as sodium citrate or dicalcium phosphate, and/or any of the following: (1) fillers or extenders, (2) binders, (3) humectants, (4) disintegrating agents, (5) solution retarding agents, (6) absorption accelerators, (7) wetting agents, (8) absorbents, (9) lubricants, (10) complexing agents, and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using suitable excipients. The pharmaceutical compositions according to the present invention may contain conventional pharmaceutical carriers and/or auxiliary agents. In some embodiments, he pharmaceutical compositions according to the present invention may contain conventional carrier agents including a binder, a lubricant and/or a glidant selected from those products and materials generally used in pharmaceutical industry for preparation of pharmaceutical compositions for an intended route of administration.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

Liquid dosage forms useful for oral administration include pharmaceutically acceptable carriers and the active ingredient provided as a solid form for reconstitution prior to administration or as a liquid (e.g., solutions, suspensions, or emulsions). In addition to the active ingredient, a liquid dosage forms may contain inert diluents commonly used in the art. For example, formulations of pharmaceutically acceptable compositions for injection can include aqueous solutions such as water or physiologically buffered saline or other solvents or vehicles suitable for the intended route of administration. In some embodiments, the pharmaceutical composition is formulated for parenteral administration.

The therapeutically effective amount of a pharmaceutical composition can be determined by human clinical trials to determine the safe and effective dose for a patient with a relevant diagnosis. It is generally understood that the effective amount of the compound may vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the pharmaceutical composition at a dose and dose interval determined to be safe and effective for the patient.

The present disclosure includes the use of pharmaceutically acceptable salts of compounds of the invention in the compositions and methods of the present invention. Pharmaceutically-acceptable salts include, for example, acid-addition salts and base-addition salts. The acid that is added to a compound to form an acid-addition salt can be an organic acid or an inorganic acid. A base that is added to a compound to form a base-addition salt can be an organic base or an inorganic base. In some embodiments, a pharmaceutically-acceptable salt is a metal salt, in some embodiments, a pharmaceutically-acceptable salt is an ammonium salt. For example, a pharmaceutically acceptable acid addition salt can exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

In some embodiments, a compound of Formula (I) can provide beneficial properties. For example, the compounds described herein may provide beneficial therapeutic properties while minimizing emesis. For example, compounds of Formula (I) may have improved selectivity for inhibiting PDE4 and the specific variants thereof. In some embodiments, the compounds of Formula (I) described herein inhibit specific variants of PDE4. In certain instances, preferred compounds will have PDE4 isoform selectivity with a 10-fold bias for one or more isoforms over the others in-class. For example, PDE4b selective and PDE4d selective compounds are desirable. Furthermore, compounds that have a high brain exposure with brain:plasma ratios (expressed as Kp)>0.3 and ideally >0.7 are most desirable. In some embodiments, compounds of Formula (I) can be selected to provide beneficial properties. For example, compounds can have improved the selectivity for inhibiting PDE4 compared to SERT. In some embodiments, compounds disclosed herein are at least 2×, at least 3×, at least 5×, or at least 10× selective for PDE4 over SERT.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups, C₁-C₁₀ branched-chain alkyl groups, cycloalkyl (alicyclic) groups, alkyl-substituted cycloalkyl groups, and cycloalkyl-substituted alkyl groups. Preferably, the “alkyl” group refers to C₁-C₇ straight-chain alkyl groups or C₁-C₇ branched-chain alkyl groups. Most preferably, the “alkyl” group refers to C₁-C₃ straight-chain alkyl groups or C₁-C₃ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “haloalkyl” refers to an alkyl group substituted with at least one hydrogen atom on a carbon replaced by a halogen. Illustrative halogens include fluoro, chloro, bromo, and iodo. Illustrative haloalkyl groups include trifluoromethyl and 2,2,2-trifluoroethyl, etc.

The term “alkoxyalkyl” refers to an alkyl group substituted with an alkoxy group and may be represented by the general formula alkyl-O-alkyl.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

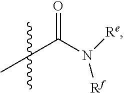

wherein R^{e} and R^{f} each independently represent a hydrogen or hydrocarbyl group, or R^{e} and R^{f} taken together with the N atom to which they are attached complete a heterocycle having from 4 to 8 atoms in the ring structure.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Preferably, the “alkoxy” group refers to C₁-C₇ straight-chain alkoxy groups or C₁-C₇ branched-chain alkoxy groups. Representative alkoxy groups include methoxy, ethoxy, propoxy, tert-butoxy and the like.

The term “aryloxy” refers to an aryl group having an oxygen attached thereto. Preferably, the “aryloxy” group refers to C₆-C₁₀ aryloxy groups or 5-7-membered heteroaryloxy groups. Representative aryloxy groups include phenoxy (C₆H₅—O—) and the like.

The terms “amine” and “amino” are art-recognized and refer to both unsubstituted and substituted amines and salts thereof, e.g., a moiety that can be represented by

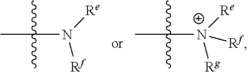

wherein R^{e}, R^{f}, and R^{g}, each independently represent a hydrogen or a hydrocarbyl group, or R^{e} and R^{f} taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring, for example a phenyl. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

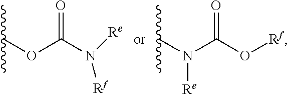

wherein R^{e} and R^{f} independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains six or fewer carbon atoms, preferably four or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

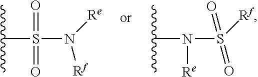

wherein R^{e} and R^{f} independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR^{e} or —SC(O)R^{e}
wherein R^{e} represents a hydrocarbyl.

The term “thioether”, as used herein, is equivalent to an ether, wherein the oxygen is replaced with a sulfur.

The term “urea” is art-recognized and may be represented by the general formula

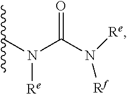

wherein R^{e} and R^{f} independently represent hydrogen or a hydrocarbyl.

The term “modulate” as used herein includes the inhibition or suppression of a function or activity (such as cell proliferation) as well as the enhancement of a function or activity.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form mesembrenone. Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs include using ester or phosphoramidate as biologically labile or cleavable (protecting) groups. The prodrugs of this disclosure are metabolized to produce mesembrenone. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

### Alternative Embodiments

In some embodiments, compounds described herein are compounds of formula (I): are compounds of formula (I):

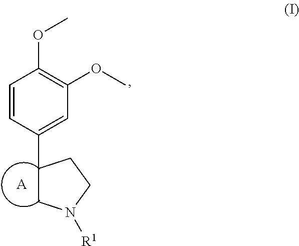

or a pharmaceutically acceptable salt thereof, wherein
   R¹ is H or C₁-C₇ alkyl; and
   ring A is

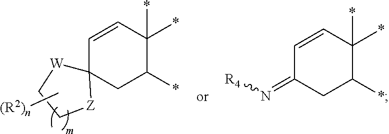

wherein * denotes the attachment points of ring A to the compound of formula (I), and wherein
   each of W and Z is independently O, NH, or S;
   each R² is independently C₁-C₃ alkyl, —COOH, —CONH₂, phenyl, 5- to 7-membered heterocyclyl, or 5- to 7-membered heteroaryl, wherein each hydrogen atom in phenyl, 5- to 7-membered heterocyclyl and 5- to 7-membered heteroaryl is optionally substituted by —OH, C₁-C₃ alkyl, C₁-C₃ alkoxy, C₁-C₃ alkanol, or —NO₂, or two R⁴s on a single carbon atom combine to form ═O; or two adjacent R⁴s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol;
   m is 1 or 2;
   n is 0, 1, or 2; and
   R⁴ is OH, C₁-C₆ alkoxy, or —NHC(O)-5- to 7-membered heteroaryl, wherein each hydrogen atom in 5- to 7-membered heteroaryl is optionally substituted by —OH, C₁-C₃ alkyl, or C₁-C₃ alkanol.

In certain embodiments, the compound is of formula (I-1):

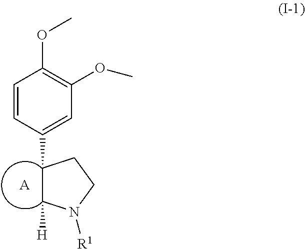

or a pharmaceutically acceptable salt thereof, wherein
   R¹ is H or C₁-C₇ alkyl; and
   ring A is

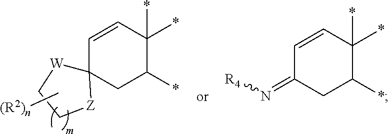

wherein * denotes the attachment points of ring A to the compound of formula (I), and wherein
each of W and Z is independently O, NH, or S;
each R² is independently C₁-C₃ alkyl, —COOH, —CONH₂, phenyl, 5- to 7-membered heterocyclyl, or 5- to 7-membered heteroaryl, wherein each hydrogen atom in phenyl, 5- to 7-membered heterocyclyl and 5- to 7-membered heteroaryl is optionally substituted by —OH, C₁-C₃ alkyl, C₁-C₃ alkoxy, C₁-C₃ alkanol, or —NO₂, or two R⁴s on a single carbon atom combine to form ═O; or two adjacent R⁴s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol;
m is 1 or 2;
n is 0, 1, or 2; and
   R⁴ is OH, C₁-C₆ alkoxy, or —NHC(O)-5- to 7-membered heteroaryl, wherein each hydrogen atom in 5- to 7-membered heteroaryl is optionally substituted by —OH, C₁-C₃ alkyl, or C₁-C₃ alkanol; and
   the compound of formula (I-1) has the absolute stereochemistry shown.

In certain embodiments, the compound of formula (I) is a compound of formula (IIb):

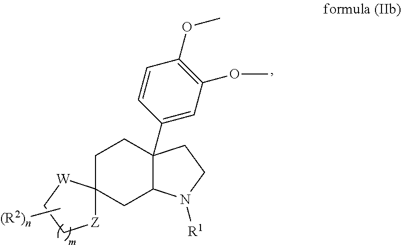

or a pharmaceutically acceptable salt thereof, wherein R¹, R², m, n, W, and Z are as defined herein.

In certain embodiments, the compound is of formula (IIb):

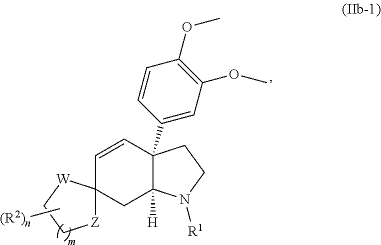

or a pharmaceutically acceptable salt thereof wherein R¹, R², m, n, W, and Z are as defined herein; and the compound of formula (IIb-1) has the absolute stereochemistry shown.

In certain embodiments, the compound of formula (I) is a compound of formula (IVb):

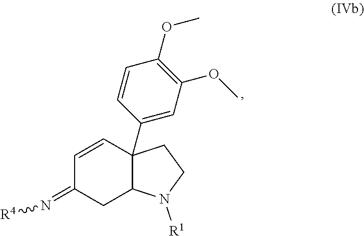

or a pharmaceutically acceptable salt thereof, wherein R¹ and R⁴ are as defined herein.

In certain embodiments, the compound is of formula (IVb-1):

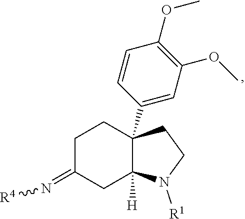

formula (IVb-1), or a pharmaceutically acceptable salt thereof, wherein R¹ and R⁴ are as defined herein.

In certain embodiments, the compound is of formula (IA):

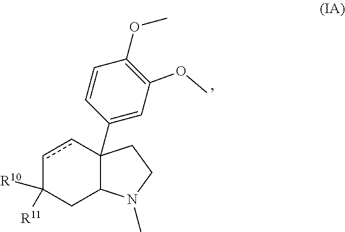

or a pharmaceutically acceptable salt thereof, wherein the dashed bond is absent or present, and R¹⁰ and R¹¹ are as defined herein, for example as a biologically labile moiety selected to provide in vivo conversion of a compound of Formula (IA) to mesembrenone.

In some embodiments, the compound is selected from the group consisting of:

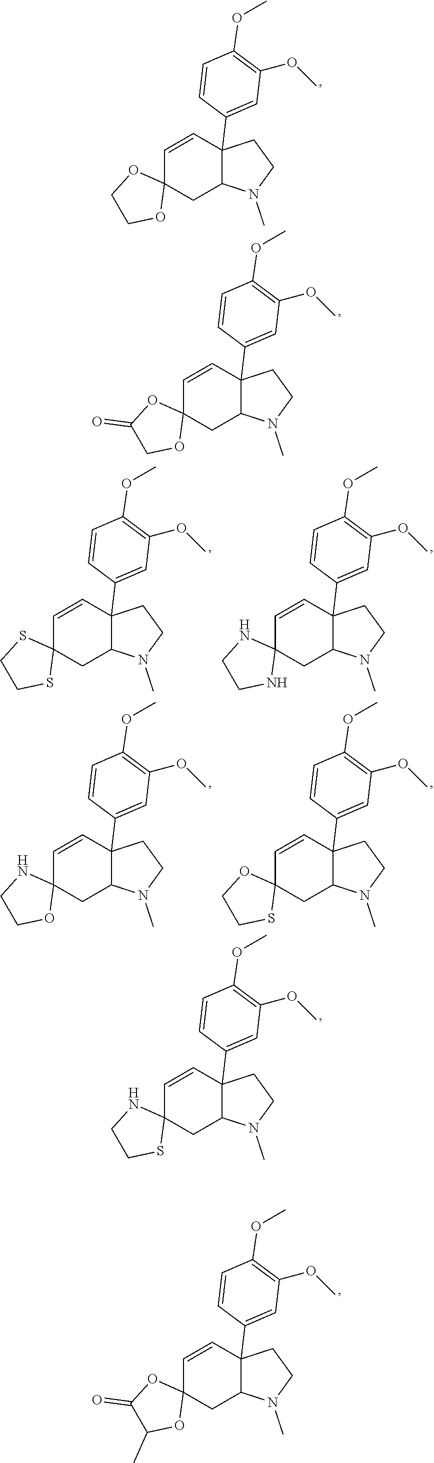

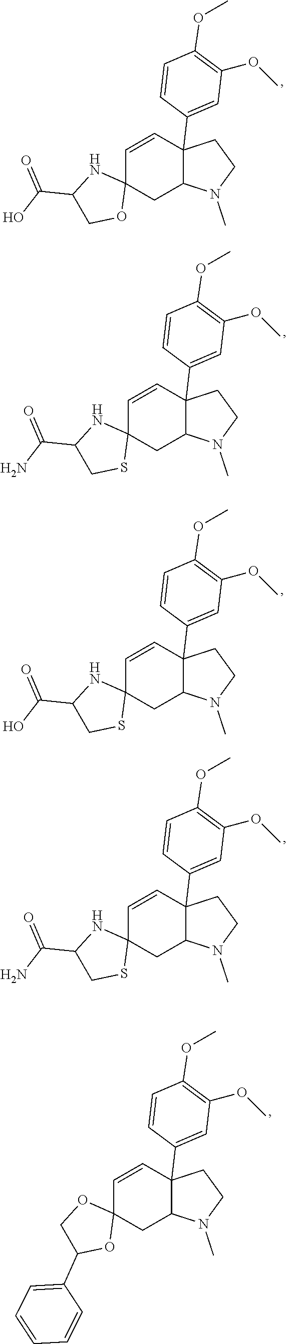

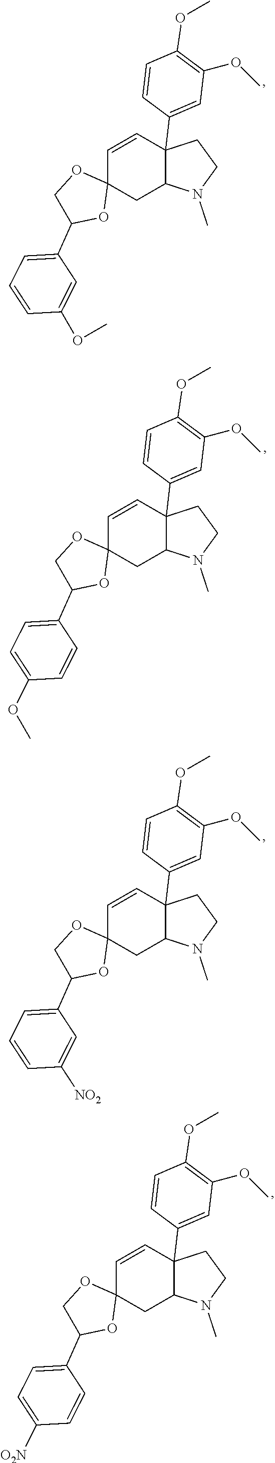 [SMILES: COc1cccc(C2COC3(C=CC4(c5ccc(OC)c(OC)c5)CCN(C)C4C3)O2)c1 ; COc1ccc(C2COC3(C=CC4(c5ccc(OC)c(OC)c5)CCN(C)C4C3)O2)cc1 ; COc1ccc(C23C=CC4(CC2N(C)CC3)OCC(c2cccc([N+](=O)[O-])c2)O4)cc1OC ; COc1ccc(C23C=CC4(CC2N(C)CC3)OCC(c2ccc([N+](=O)[O-])cc2)O4)cc1OC]

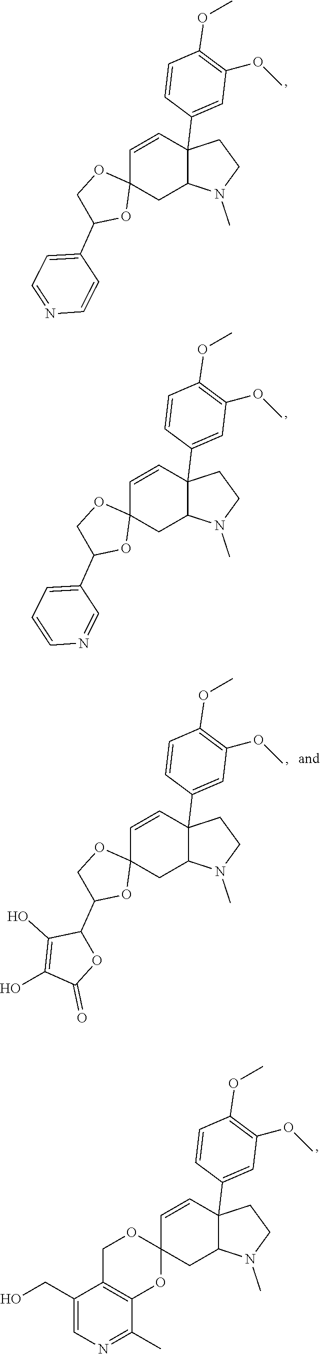 [SMILES: COc1ccc(C23C=CC4(CC2N(C)CC3)OCC(c2ccncc2)O4)cc1OC ; COc1ccc(C23C=CC4(CC2N(C)CC3)OCC(c2cccnc2)O4)cc1OC ; COc1ccc(C23C=CC4(CC2N(C)CC3)OCC(C2OC(=O)C(O)=C2O)O4)cc1OC ; COc1ccc(C23C=CC4(CC2N(C)CC3)OCc2c(CO)cnc(C)c2O4)cc1OC]

or a pharmaceutically acceptable salt thereof.

In some embodiments, the compound is selected from the group consisting of:

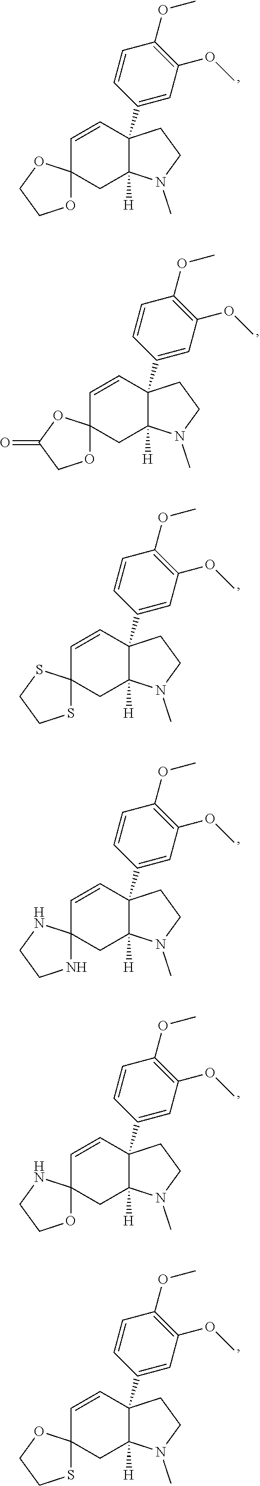

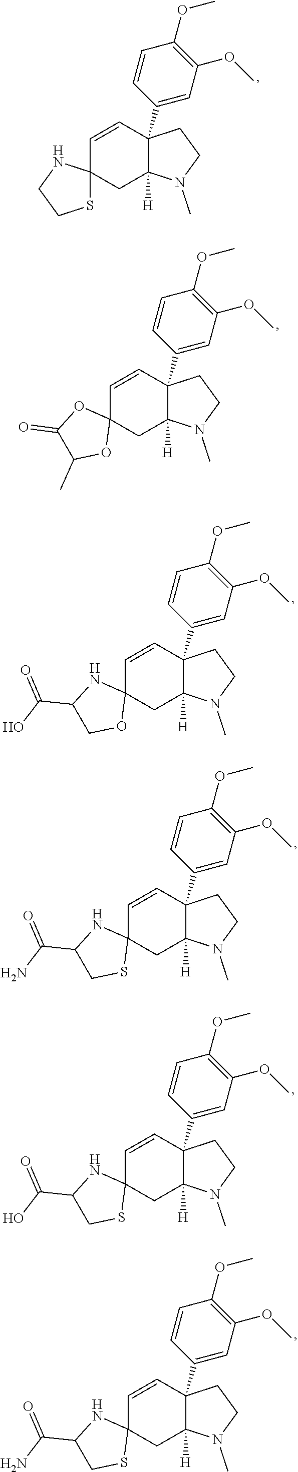

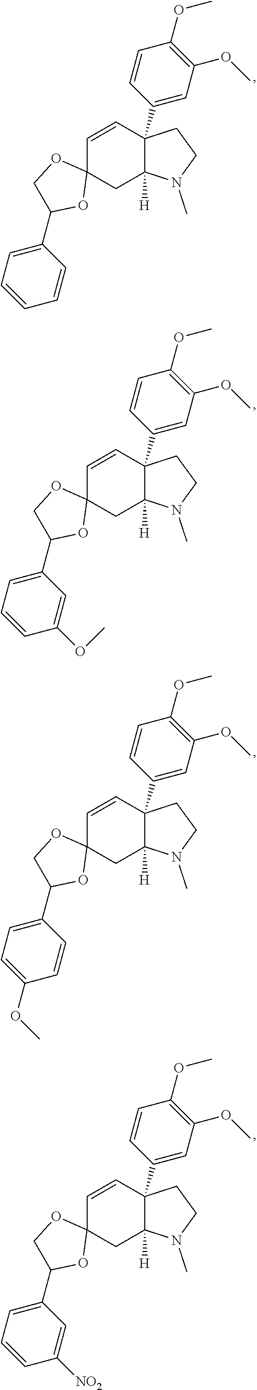 [SMILES: COc1ccc([C@@]23C=CC4(C[C@@H]2N(C)CC3)OCC(c2ccccc2)O4)cc1OC ; COc1cccc(C2COC3(C=C[C@@]4(c5ccc(OC)c(OC)c5)CCN(C)[C@H]4C3)O2)c1 ; COc1ccc(C2COC3(C=C[C@@]4(c5ccc(OC)c(OC)c5)CCN(C)[C@H]4C3)O2)cc1 ; COc1ccc([C@@]23C=CC4(C[C@@H]2N(C)CC3)OCC(c2cccc([N+](=O)[O-])c2)O4)cc1OC]

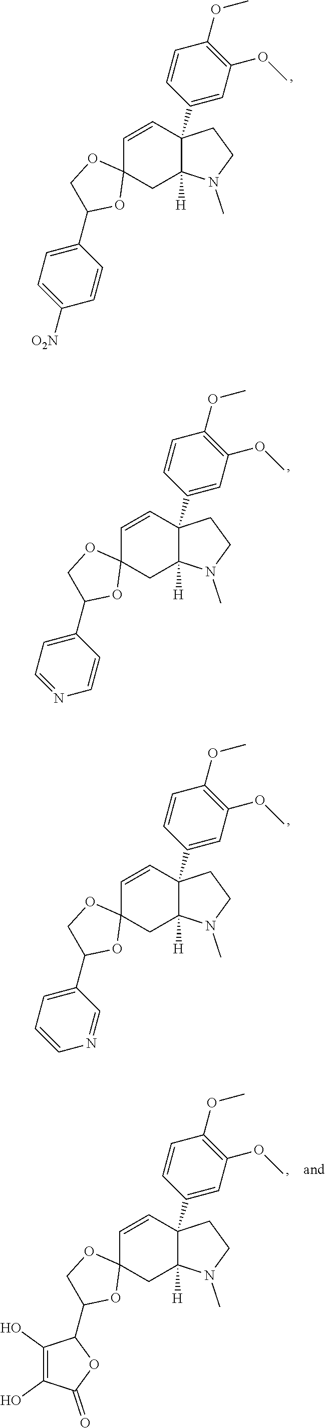 [SMILES: COc1ccc([C@@]23C=CC4(C[C@@H]2N(C)CC3)OCC(c2ccc([N+](=O)[O-])cc2)O4)cc1OC ; COc1ccc([C@@]23C=CC4(C[C@@H]2N(C)CC3)OCC(c2ccncc2)O4)cc1OC ; COc1ccc([C@@]23C=CC4(C[C@@H]2N(C)CC3)OCC(c2cccnc2)O4)cc1OC ; COc1ccc([C@@]23C=CC4(C[C@@H]2N(C)CC3)OCC(C2OC(=O)C(O)=C2O)O4)cc1OC]

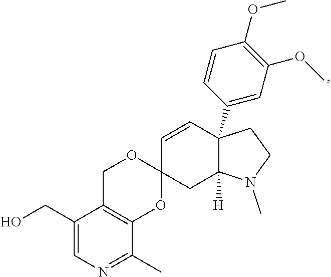 [SMILES: COc1ccc([C@@]23C=CC4(C[C@@H]2N(C)CC3)OCc2c(CO)cnc(C)c2O4)cc1OC]

or a pharmaceutically acceptable salt thereof, and the compound has the absolute stereochemistry shown.

In certain embodiments, the compounds described herein convert to mesembrenone as measured by HPLC after 24 hours at a pH of 2 (0.01 M HCl) and a temperature of 37° C. in the Hydrolysis Assay of Example A1.

The present application relates to compounds that can be converted to mesembrenone under conditions encountered within the body, such as upon oral administration. In some embodiments, compounds are provided that hydrolyze to form mesembrenone under acidic conditions (e.g., pH 2) at 37° C. In addition, Applicant has discovered compounds useful for isolating stable forms of individual stereoisomers of mesembrenone.

The invention is based in part on the discovery of compounds having useful and markedly different from naturally occurring mesembrenone, but that can be converted to mesembrenone under biologically relevant conditions. Certain compounds provided herein convert to mesembrenone under physiologically relevant conditions, using the Hydrolysis Assay of Example A1. In some embodiments, other compounds provided herein can form mesembrenone under biologically relevant acidic conditions. For example, in some embodiments, certain compounds provided herein convert to mesembrenone at acidic pH and temperatures between room temperature and human body temperature (e.g., Compound 15 converted to mesembrenone at acidic pH 2.0 in the Hydrolysis Assay at temperatures of 25 or 37° C., as provided in the data in Example A1). In some embodiments, certain compounds provided herein convert to mesembrenone at acidic pH and temperatures above room temperature including at human body temperature. In contrast, the naturally occurring (+)/(−) mesembrenone (herein “Compound 016”) did not further hydrolyze under a variety of biologically relevant conditions ranging from acidic (pH 2.0 in 0.01 M HCl) to neutral buffered conditions (pH 7.4 in 20 mM PBS) from room temperature (25° C.) to elevated temperature (40° C.) (in the Hydrolysis Assay described in Example A1).

In some embodiments, compounds are provided that permit the separation and isolation of stable form of the (+) mesembrenone separated from the naturally occurring (−) form of (−) mesembrenone.

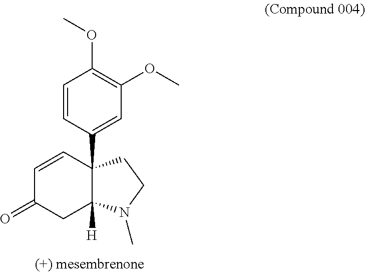 [SMILES: COc1ccc([C@]23C=CC(=O)C[C@H]2N(C)CC3)cc1OC]

or a pharmaceutically acceptable salt thereof.

The Examples provide non-limiting examples of reactions of racemic and (+) and (−) mesembrenone with various reactive compounds to obtain compounds disclosed herein.

In some embodiments, a method of isolating stable forms of (+) mesembrenone and (−) mesembrenone is provided.

In certain embodiments, compounds described herein can form mesembrenone (e.g., (−) mesembrenone) under biologically relevant conditions. For example, in some embodiments, compounds of disclosed herein (e.g., compounds of Formula (I)) can hydrolyze in highly acidic environments (e.g., pH of about 2 at room temperature or more comparably stringent conditions typically encountered within the alimentary canal of a mammal) at a rate that is advantageous for providing a desired bioabsorption (% F) following oral administration by a mammal and leading to a desired pharmacokinetic profile of mesembrenone (e.g., (−) mesembrenone) to the mammal.

In some embodiments, a compound according to the present disclosure is of formula (I):

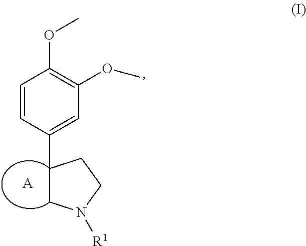

or a pharmaceutically acceptable salt thereof.

In some embodiments, ring A is

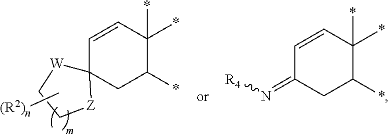

wherein * denotes the attachment points of ring A to the compound of formula (I).

In some embodiments, each of W and Z is independently O, NH, or S. In some embodiments, W is O. In some embodiments, W is NH. In some embodiments, W is S.

In some embodiments, each R² is independently C₁-C₃ alkyl, —COOH, —CONH₂, phenyl, 5- to 7-membered heterocyclyl, or 5- to 7-membered heteroaryl. In some embodiments, each hydrogen atom in phenyl, 5- to 7-membered heterocyclyl and 5- to 7-membered heteroaryl is optionally substituted by —OH, C₁-C₃ alkyl, C₁-C₃ alkoxy, C₁-C₃ alkanol, or —NO₂. In some embodiments, R² is —COOH, —CONH₂, phenyl, 5- to 7-membered heterocyclyl, or 5- to 7-membered heteroaryl, wherein each hydrogen atom in phenyl, 5- to 7-membered heterocyclyl and 5- to 7-membered heteroaryl is optionally substituted by —OH, C₁-C₃ alkyl, C₁-C₃ alkoxy, C₁-C₃ alkanol, or —NO₂. In some embodiments, R² is phenyl, wherein each hydrogen atom in phenyl is optionally substituted by —OH, C₁-C₃ alkyl, C₁-C₃ alkoxy, or —NO₂. In some embodiments, R² is 5- to 7-membered heterocyclyl or 5- to 7-membered heteroaryl, wherein each hydrogen atom in 5- to 7-membered heterocyclyl and 5-to 7-membered heteroaryl is optionally substituted by —OH, C₁-C₃ alkyl, C₁-C₃ alkoxy, C₁-C₃ alkanol, or —NO₂. In some embodiments, R² is pyridyl. In some embodiments, R² is

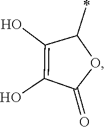 [SMILES: *C1OC(=O)C(O)=C1O]

wherein * denotes the point of attachment of R² the compound. In some embodiments, R² is —COOH or —CONH₂.

In some embodiments, two R²s on a single carbon atom combine to form ═O.

In some embodiments, two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol. In some embodiments, the two R²s together with the carbon atoms to which they are attached combine to form pyridyl, wherein each hydrogen atom in pyridyl is optionally substituted by —OH, C₁-C₃ alkyl, or C₁-C₃ alkanol.

In some embodiments, m is 1 or 2.

In some n is 0, 1, or 2. In some embodiments, n is 0. In some embodiments, n is 1. In some embodiments, n is 2.

In some embodiments, R³ is —OSi(C₁-C₆ alkyl)₃ or —OC(O)C₁-C₆ alkyl. In some embodiments, R³ is —OSi(C₁-C₆ alkyl)₃. In some embodiments, R³ is —OC(O)C₁-C₆ alkyl.

In some embodiments, R⁴ is OH, C₁-C₆ alkoxy, or —NHC(O)-5- to 7-membered heteroaryl. In some embodiments, each hydrogen atom in 5- to 7-membered heteroaryl is optionally substituted by —OH, C₁-C₃ alkyl, or C₁-C₃ alkanol.

In some embodiments, R¹ is H or C₁-C₇ alkyl. In some embodiments, R¹ is C₁-C₃ alkyl. In some embodiments, R¹ is methyl.

In some embodiments, the compounds of Formula (I) can be obtained by reacting mesembrenone (M) with a compound designated as Generally Recognized as Safe (GRAS) by the U.S. Food and Drug Administration (FDA) (G) under sections 201(s) and 409 of the U.S. federal Food, Drug and Cosmetics Act (FDCA), and under the corresponding implementing regulations in 21 CFR 170.3 and 21 CFR 170.30. For example, novel compounds that convert to mesembrenone can be obtained by reaction of either compound with lactic acid, glycolic acid, ascorbic acid (vitamin C), and pyridoxine (vitamin B6) derivatives. Table 2 provides examples of compounds that can be reacted to obtain compounds of Formula (I) by the reaction: Compound M+Compound R→Compound C.

TABLE 2

| Reactant M | Reactant R | Product Compound C |
|---|---|---|
| 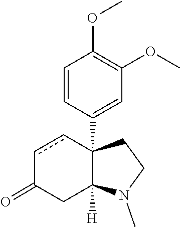 | 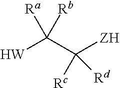 | 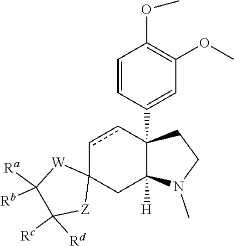 |
| 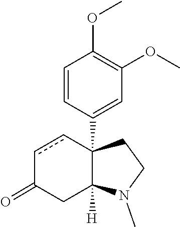 | 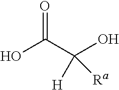 | 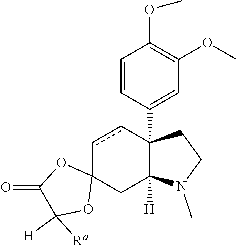 |
| 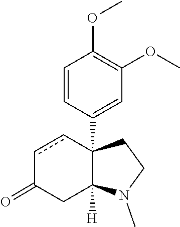 | 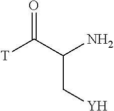 | 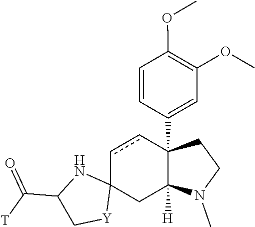 |
| 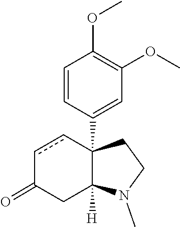 | 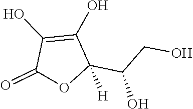 [SMILES: O=C1O[C@H]([C@@H](O)CO)C(O)=C1O] | 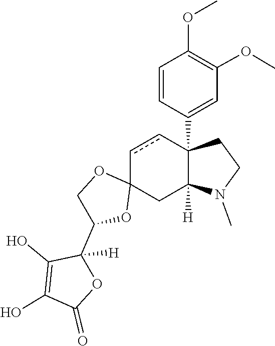 |
| 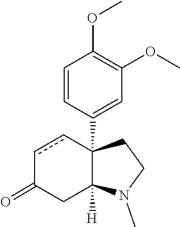 | 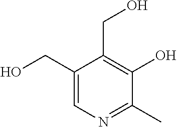 | 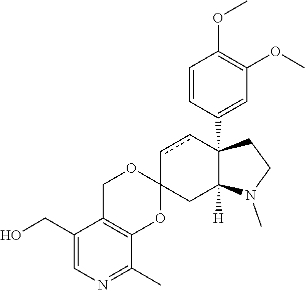 |
| 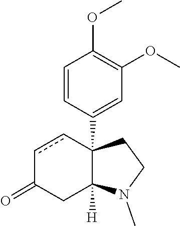 | (R⁸)₂O or R⁸ —X | 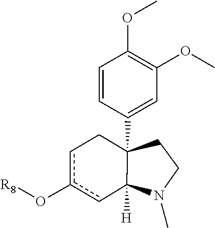 |
| 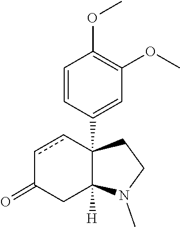 | 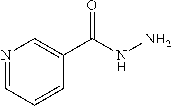 | 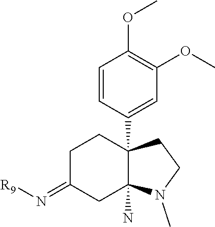 |

wherein the dashed bond is present or absent.

Unless otherwise indicated in the tables of compounds herein, the abbreviation RAC or rac indicates a racemic mixture, and DIAST indicates a specific diastereomer. In illustrative embodiments, although a compound may be depicted with [symbol] or [symbol] bonds, such a depiction may be denoting relative stereochemistry based on elution peaks from a chiral separation.

Compounds of Formula (I) including compounds of Formula (IIa) and Formula (IIb) can be prepared as described with respect to exemplary compounds in the examples below. In general, treatment of a ketone, such as mesembrenone, with an appropriate diol or dithiane, and an acid catalyst, such as p-toluenesulfonic acid or methanesulfonic acid, in a solvent, such as toluene, at an elevated temperature is a method to prepare compounds of Formula (IIa-IIb).

TABLE
Exemplary Compounds of Formula Ila-IIb

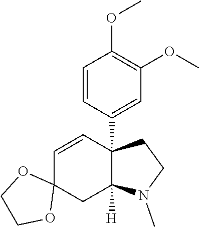 [SMILES: COc1ccc([C@@]23C=CC4(C[C@@H]2N(C)CC3)OCCO4)cc1OC]

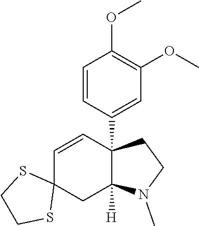 [SMILES: COc1ccc([C@@]23C=CC4(C[C@@H]2N(C)CC3)SCCS4)cc1OC]

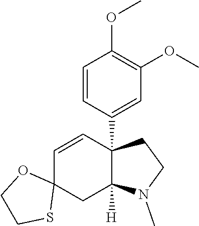 [SMILES: COc1ccc([C@@]23C=CC4(C[C@@H]2N(C)CC3)OCCS4)cc1OC]

In some embodiments, the compound of formula (I) is a compound of formula (IX):

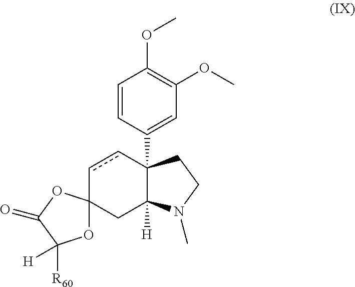

or a pharmaceutically acceptable salt thereof, wherein the dashed bond is absent or present, and R₆₀ is hydrogen or methyl.

In some embodiments, a compound of Formula (IX) can be a compound of Formula (IX-2) that converts to mesembrenone (e.g.. (−) mesembrenone) upon oral administration to a mammal and upon hydrolysis in sufficiently acidic conditions (e.g., pH 2 at 37 degrees C.), where R₆₀ is as defined with respect to Formula (I-B) above.

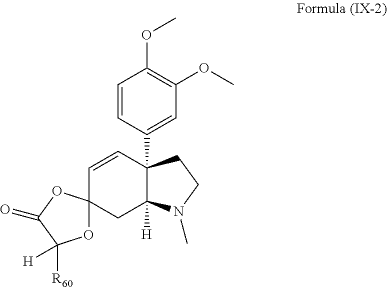

Compounds of Formula (IX) including compounds of Formula (IX-2) can be prepared as described with respect to compounds in the examples below. In general treatment of a ketone, such as mesembrenone, with an appropriate alpha-hydroxy carboxylic acid, with a Lewis acid, such as boron trifluoride etherate, in a solvent, such as dichloromethane, is a method to prepare compounds of Formula (IX). A compound of formula (IX-2) where R₆₀ is methyl can be obtained by reacting mesembrenone with lactic acid (e.g., with TsOH, toluene). In some embodiments, the compound can be a lactate or glycolate derivative of mesembrenone. R₆₀ in Scheme 2 is as defined above with respect to Formula (I-B).

Table provides non-limiting examples of certain compounds of Formula (IX).

TABLE
Exemplary Compounds of Formula IX

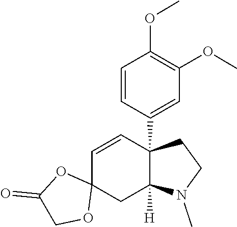 [SMILES: COc1ccc([C@@]23C=CC4(C[C@@H]2N(C)CC3)OCC(=O)O4)cc1OC]

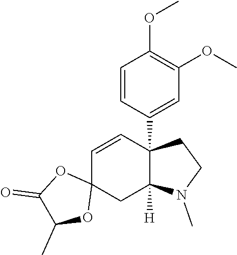

In some embodiments, the compound of Formula (I) is a compound of Formula (X) or a pharmaceutically acceptable salt thereof:

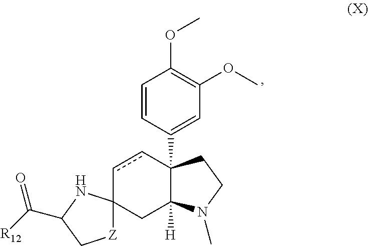

wherein the dashed bond is absent or present, Z is S or O; and R₁₂ is hydroxyl or amino.

In some embodiments, a compound of Formula (I-C) can be a compound of Formula (I-C-2) that converts to mesembrenone (e.g., (−) mesembrenone) upon oral administration to a mammal and upon hydrolysis in sufficiently acidic conditions (e.g., pH 2 at 37° C.), where Z and R₁₂ are as defined with respect to Formula (I-C) above.

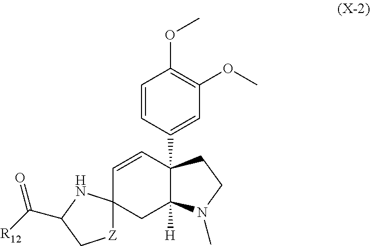

Compounds of Formula (X) including compounds of Formula (X-2) can be prepared as described with respect to exemplary compounds in the examples below. In general, treatment of a ketone with an appropriate alpha-amino carboxylic acid or alpha-amino amide, in a solvent, such as ethanol, at an elevated temperature is a method to prepare compounds of Formula (X).

In some embodiments, the compound of Formula (I) is a compound of Formula (XI) or a pharmaceutically acceptable salt thereof:

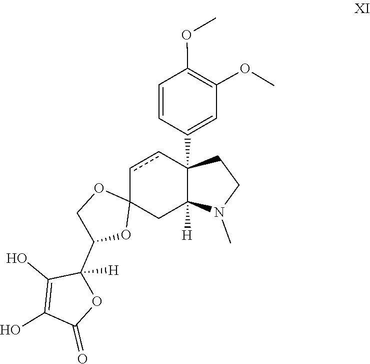

wherein the dashed bond is absent or present.

In some embodiments, a compound of Formula (XI) can be a compound of Formula (XI-2) that converts to mesembrenone (e.g., (−) mesembrenone) upon oral administration to a mammal and upon hydrolysis in sufficiently acidic conditions (e.g., pH 2 at 37° C.). In some embodiments, the compound can be an ascorbate derivative of mesembrenone.

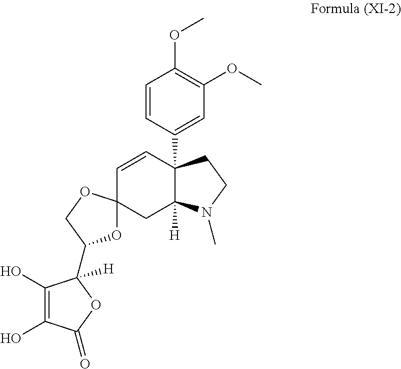

Compounds of Formula (XI) including compounds of Formula (XI-2) can be prepared as described with respect to compounds in the examples below. In general, treatment of a ketone such as mesembrenone, with a naturally occurring diol, such as ascorbic acid, with an acid catalyst in a solvent, such as toluene, is a method to prepare compounds of Formula (XI). In some embodiments, mesembrenone can be reacted with ascorbic acid (e.g., TsOH, acetone) to obtain a compound of Formula (XI-2).

Table provides non-limiting examples of certain compounds of Formula (XI).

Table. Exemplary Compounds of Formula XI

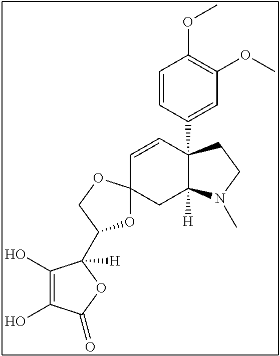

In some embodiments, the compound of Formula (I) is a compound of Formula (XII) or a pharmaceutically acceptable salt thereof:

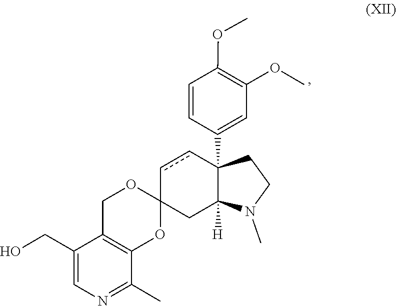

wherein the dashed bond is absent or present.

In some embodiments, a compound of Formula (I-D) can be a compound of Formula (XII-2) that converts to mesembrenone (e.g., (−) mesembrenone) upon oral administration to a mammal and upon hydrolysis in sufficiently acidic conditions (e.g., pH 2 at 37° C.).

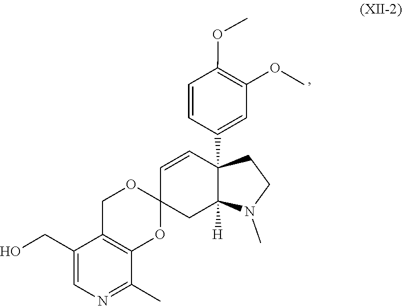 [SMILES: COc1ccc([C@@]23C=CC4(C[C@@H]2N(C)CC3)OCc2c(CO)cnc(C)c2O4)cc1OC]

Compounds of Formula (XII) including compounds of Formula (I XII-2) can be prepared as described with respect to exemplary compounds in the examples below. In general treatment of a ketone such as mesembrenone, with a compound such as pyridoxine or an analog thereof (e.g., a vitamin B₆ analog), with an acid catalyst in a solvent is a method to prepare compounds of Formula (XII).

Table provides non-limiting examples of certain compounds of Formula (XII). Table. Exemplary Compounds of Formula XII

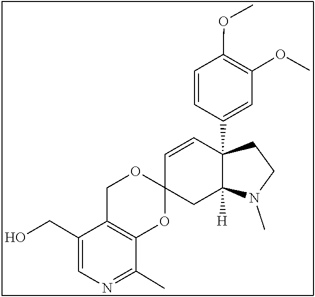 [SMILES: COc1ccc([C@@]23C=CC4(C[C@@H]2N(C)CC3)OCc2c(CO)cnc(C)c2O4)cc1OC]

In some embodiments, the compound is a compound of Formula (XV) or a pharmaceutically acceptable salt thereof

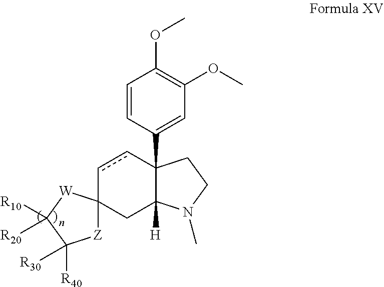

wherein
   the dashed bond is absent or present;
   n is 0 or 1,
   W and Z are each independently CH₂, O, S or NH, provided that at least one of W or Z is O, S or NH; and
   R₁₀, R₂₀, R₃₀ and R₄₀ are each hydrogen.

In some embodiments, W and Z in Formula (XV) are each O. In some embodiments, W and Z in Formula (XV) are each S. In some embodiments, W and Z in Formula (XV) are each NH. In some embodiments, W is NH and Z is S in Formula (XV). In some embodiments, W is O and Z is S in Formula (XV). In some embodiments, W is CH₂ and Z is O in Formula (XV). In some embodiments, W is CH₂ and Z is NH in Formula (XV).

In some embodiments, a compound of a compound of Formula (XV-2):

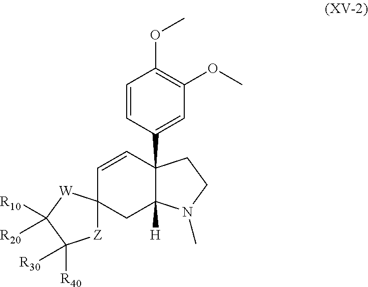

Compounds of Formula (XV) including compounds of Formula (XV-1) and Formula (XV-2) can be prepared as described with respect to exemplary compounds in the examples below.

A total synthesis of (±)-mesembrine has also been reported (Jeffs P. Sceletium alkaloids. In: Jeffs P. The Alkaloids: Chemistry and Physiology. Vol 19. New York, NY: Academic Press; 1981:1-80). Mesembrine alkaloids have been synthesized in a manner similar to that of Amaryllidaceae alkaloids (e.g., Roe C, Sandoe E J, Stephenson G R, Anson CE. Stereoselectivity in the organoiron-mediated synthesis of (±)-mesembrine. Tetrahedron Lett. 2008; 49(4):650-653; and Shamma M, Rodriguez HR. The synthesis of (+)-mesembrine. Tetrahedron. 1968; 24(22):6583-6589.5714008).

In some embodiments, many prodrugs of mesembrenone (e.g., (−) mesembrenone) are disclosed herein. Illustratively, converting mesembrenone to a prodrug can be performed by modifying the ketone on the fused ring. In some embodiments, the modification can be take the form of a protecting group. Illustrative ketone protecting groups are known in the art as described in Greene's Protective Groups in Organic Synthesis, fourth edition, the disclosure of which is hereby incorporated by reference. Illustrative ketone protecting groups include acyclic ketals, cyclic ketals, chiral ketals, dithio ketals, cyclic dithio ketals, monothiol ketals, cyclic monothiol ketals, cyanohydrins, hydrazones, oximes, pyrrole carbinols, O-silylimdazoyl aminals, cyclic aminals, and benzothiazoles. Ketones may also be protected by protecting the enolate, for example by trimethylsilyl enol ethers, and enamines.

In some embodiments, a method of isolating stable forms of (+) mesembrenone and (−) mesembrenone is provided. In illustrative embodiments, the method minimizes racemization of mesembrenone. In some embodiments, stereoisomer analogs of (+) mesembrenone and (−) mesembrenone can be formed. The (+)/(−) mesembrenone analogs can then be separated. The (+) analog can then be converted to (+) mesembrenone. The (−) analog can then be converted to (−) mesembrenone. The conversion can be performed by hydrolysis, preferably in the presence of an acid.

In illustrative embodiments, a method of extending the pharmacokinetic properties of mesembrenone is described. In illustrative embodiments, the pharmacokinetic properties of mesembrenone is extended by forming a prodrug, for example by modifying the ketone on the fused ring.

In some embodiments, compounds of Formula (I-1) can form mesembrenone (e.g., (−) mesembrenone) under biologically relevant conditions, including compounds of formula (I-1), formula (IIb-1), formula (IIIb). In certain embodiments, methods of administering a therapeutic alkaloid compound comprise the oral administration of a compound of formula (I-1), formula (IIb-1), and formula (IIIb).

In some embodiments, methods of treating a patient suffering from a disease comprise administering to a patient a composition comprising a compound disclosed herein for the treatment or prevention of a mental health disorder. In some embodiments, methods of treating a patient suffering from a disease comprise administering to a patient a composition comprising a compound disclosed herein for the treatment or prevention of a diagnosed condition selected from anxiety and depression. In some embodiments, a method comprises the administration to a patient in need thereof of a therapeutically effective amount of a compound of Formula (I) for the treatment of depression. In some embodiments, a method comprises the administration to a patient in need thereof of a therapeutically effective amount of a compound of Formula (I) for the treatment of a condition selected from the group consisting of: anxiety associated with depression, anxiety with depression, mixed anxiety and depressive disorder. In some embodiments, a method comprises the administration to a patient in need thereof of a therapeutically effective amount of a compound of Formula (I) for the treatment of anxiety and hysteria or anxiety and depression.

In some embodiments, the compound disclosed herein is administered to the patient in a unit dose. In some embodiments, the compound disclosed herein is prescribed to a patient in an oral unit dose for such as a capsule or tablet once or more times per day. In some embodiments, a compound disclosed herein is administered to a patient for the treatment of a disease or condition for which mesembrenone is safe and effective for treatment. In some embodiments, a method comprises the administration to a patient in need thereof of a therapeutically effective amount of a compound of Formula (I) for the treatment of anxiety. In some embodiments, a method comprises the administration to a patient in need thereof of a therapeutically effective amount of a compound of Formula (I) for the treatment of a disease selected from the group consisting of mild to moderate depression and major depressive episodes. In some embodiments, a method comprises the administration to a patient in need thereof of a therapeutically effective amount of a compound of Formula (I) for the treatment of a disease selected from the group consisting of psychological and psychiatric disorders where anxiety is present. In some embodiments, a method comprises the administration to a patient in need thereof of a therapeutically effective amount of a compound of Formula (I) for the treatment of major depressive episodes. In some embodiments, a method comprises the administration to a patient in need thereof of a therapeutically effective amount of a compound of Formula (I) for the treatment of a disease selected from the group consisting of alcohol and drug dependence, bulimia nervosa, and obsessive-compulsive disorders. In some embodiments, an amount of from 20 micrograms to 2 milligrams of a compound of Formula (I) is orally administered to a patient to treat the patient in need thereof with a therapeutic compound selected from the group consisting (−)/(+) mesembrenone, (−) mesembrenone and (+) mesembrenone. In some embodiments, an amount of from 20 micrograms to 2 milligrams of a compound of Formula (I) is orally administered to a patient to treat the patient in need thereof with a therapeutic compound selected from (−)/(+) mesembrenone.

In certain embodiments, the compound is of formula (I):

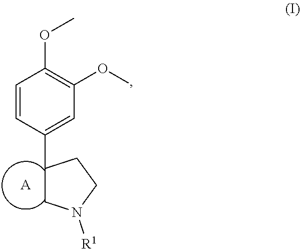

or a pharmaceutically acceptable salt thereof, wherein
R¹ is C₁-C₇ alkyl or H; and
ring A is

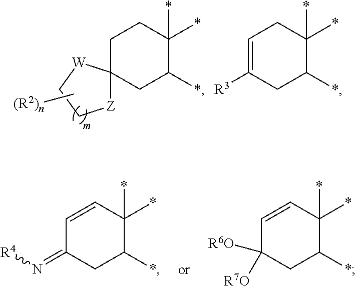

wherein * denotes the attachment points of ring A to the compound of formula (I), and wherein
   each of W and Z is independently O, NH, or S;
   each R² is independently C₁-C₃ alkyl, C2-C6 alkenyl, —COOH, —CONH₂, phenyl, 5- to 7-membered heterocyclyl, or 5- to 7-membered heteroaryl, wherein each hydrogen atom in C₁-C₃ alkyl, C2-C6 alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol m is 1 or 2,
   n is 0, 1, 2, 3 or 4;
   R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C2-C6 alkenyl, C3-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy;
   R⁴ is —OR⁵ or —N(R⁵)₂;
   each R⁵ is H, C₁-C₆ alkyl, C₃-C₆ heterocycloalkyl, C3-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C3-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C3-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C3-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C3-C₁₀ cycloalkyl optionally substituted by —COOH, phenyl, 5- to 7-membered heteroaryl, aryloxy, C₁-C6 alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl; and
   each of R⁶ and R⁷ is independently C₁-C₃ alkyl.

In certain embodiments, the compound is of formula (I):

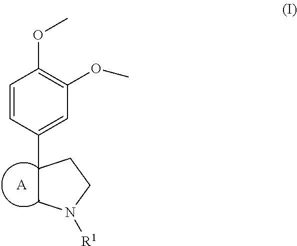

or a pharmaceutically acceptable salt thereof, wherein
   R¹ is methyl; and
   ring A is

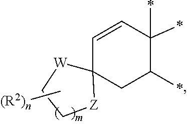

wherein * denotes the attachment points of ring A to the compound of formula (I), and wherein
   each of W and Z is independently O, NH, or S;
   each R² is independently C₁-C₃ alkyl, —COOH, —CONH₂, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂; or two R²s on a single carbon atom combine to form ═O; or two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol;
   m is 1 or 2; and
   n is 0, 1, 2, 3 or 4.

In certain embodiments, the compound is of formula (I):

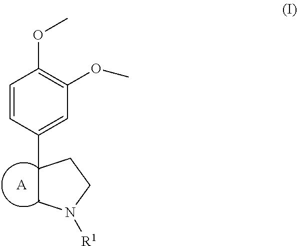

or a pharmaceutically acceptable salt thereof, wherein
   R¹ is methyl; and
   ring A is

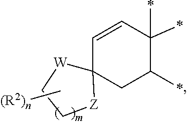

wherein * denotes the attachment points of ring A to the compound of formula (I), and wherein
   each of W and Z is independently O, NH, or S;
   m is 1 or 2;
   n is 0; or n is 1 and R² is C₁-C₃ alkyl, —COOH, —CONH₂, phenyl, or a 6-membered heterocyclyl comprising at least one nitrogen heteroatom; or n is 2, 3 or 4 and two R²s on a single carbon atom combine to form ═O and the remaining R² (if present) is C₁-C₃ alkyl;
   wherein each hydrogen atom in phenyl or 6-membered heteroaryl in R² is optionally substituted by —OH, C₁-C₃ alkyl, C₁-C₃ alkoxy, or —NO₂; and
   m is 1.

In certain embodiments, the compound is of formula (I):

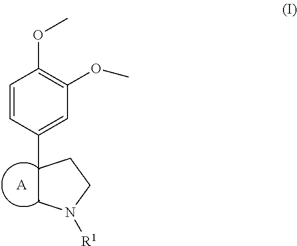

or a pharmaceutically acceptable salt thereof, wherein
   R¹ is methyl; and
   ring A is

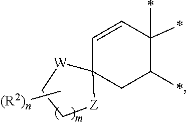

wherein * denotes the attachment points of ring A to the compound of formula (I), and wherein
   each of W and Z is independently O, NH, or S;
   m is 1; and
   n is 0; or n is 1 and R² is methyl, —COOH, —CONH₂, phenyl, or a 6-membered heterocyclyl comprising at least one nitrogen heteroatom; or n is 2, 3 or 4 with two R²s on a single carbon atom combine to form ═O and the remaining R² (if present) is selected from methyl;
   wherein each hydrogen atom in phenyl or 6-membered heteroaryl in R² is optionally substituted by —OH, methyl, methoxy, or —NO₂.

In certain embodiments, the compound is of formula (I):

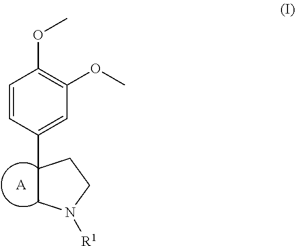

or a pharmaceutically acceptable salt thereof, wherein
   R¹ is methyl; and
   ring A is

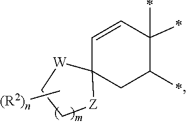

wherein * denotes the attachment points of ring A to the compound of formula (I), and wherein
   each of W and Z is independently O, NH, or S;
   m is 1; and
   n is 1 and R² is

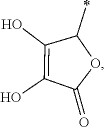 [SMILES: *C1OC(=O)C(O)=C1O]

wherein * denotes the point of attachment of R² the compound.

In certain embodiments, the compound is of formula (I):

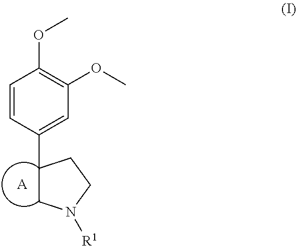

or a pharmaceutically acceptable salt thereof, wherein
   R¹ is methyl; and
   ring A is

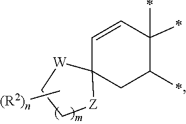

wherein * denotes the attachment points of ring A to the compound of formula (I), and wherein
   each of W and Z is independently O, NH, or S;
   m is 1; and
   n is 2, 3 or 4; and
   two adjacent R²s together with the carbon atoms to which they are attached combine to form 5- to 7-membered heteroaryl comprising a nitrogen heteroatom, wherein each hydrogen atom in 5- to 7-membered heteroaryl is optionally substituted by —OH, C₁-C₃ alkyl, or C₁-C₃ alkanol.

In certain embodiments, the compound is of formula (I):

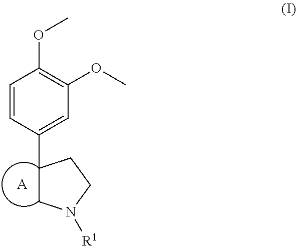

or a pharmaceutically acceptable salt thereof, wherein
   R¹ is methyl; and
   ring A is

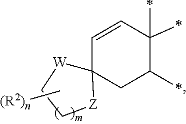

wherein * denotes the attachment points of ring A to the compound of formula (I), and wherein
   each of W and Z is independently O, NH, or S;
   m is 1; and
   n is 2, 3 or 4; and
   two adjacent R²s together with the carbon atoms to which they are attached combine to form 6-membered heteroaryl comprising a nitrogen heteroatom, wherein each hydrogen atom in 6-membered heteroaryl is optionally substituted by —OH, C₁-C₃ alkyl, or C₁-C₃ alkanol.

In certain embodiments, the compound is of formula (I):

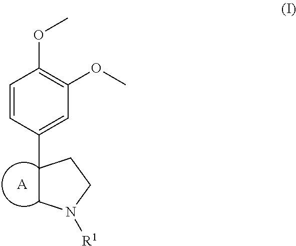

or a pharmaceutically acceptable salt thereof, wherein
   R¹ is methyl; and
   ring A is

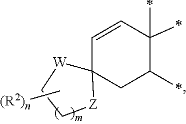

wherein * denotes the attachment points of ring A to the compound of formula (I), and wherein
   each of W and Z is independently O, NH, or S;
   m is 1; and
n is 0.

In certain embodiments, the compound is of formula (I):

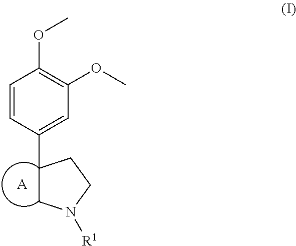

or a pharmaceutically acceptable salt thereof, wherein
   R¹ is methyl; and
   ring A is

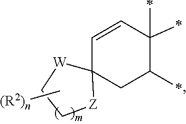

wherein * denotes the attachment points of ring A to the compound of formula (I), and wherein
   each of W and Z is independently O, NH, or S;
   m is 1; and
   n is 1 and R² is methyl, —COOH, or —CONH₂.

In certain embodiments, the compound is of formula (I):

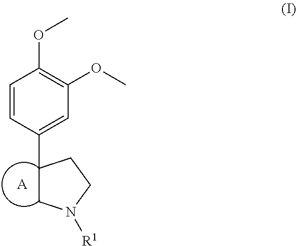

or a pharmaceutically acceptable salt thereof, wherein
   R¹ is methyl; and
   ring A is

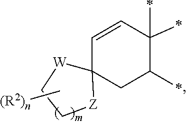

wherein * denotes the attachment points of ring A to the compound of formula (I), and wherein
   each of W and Z is independently O, NH, or S;
   m is 1; and
   n is 1 and R² is phenyl or a 6-membered heterocyclyl comprising at least one nitrogen heteroatom, wherein each hydrogen atom in phenyl or 6-membered heteroaryl in R² is optionally substituted by —OH, methyl, methoxy, or —NO₂.

In certain embodiments, the compound is of formula (I):

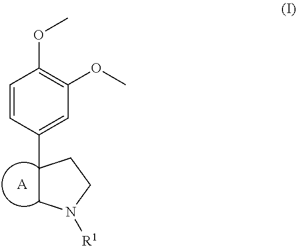

or a pharmaceutically acceptable salt thereof, wherein
   R¹ is methyl; and
   ring A is

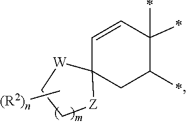

wherein * denotes the attachment points of ring A to the compound of formula (I), and wherein
   each of W and Z is independently O, NH, or S;
   m is 1; and
   n is 2 or 3 with two R²s on a single carbon atom combine to form ═O and the remaining R² (if present) is methyl;
   wherein each hydrogen atom in phenyl or 6-membered heteroaryl in R² is optionally substituted by —OH, methyl, methoxy, or —NO₂.

In certain embodiments, the compound is of formula (I):

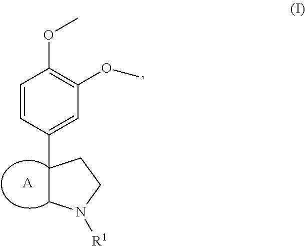

or a pharmaceutically acceptable salt thereof, wherein
R¹ is methyl; and
ring A is

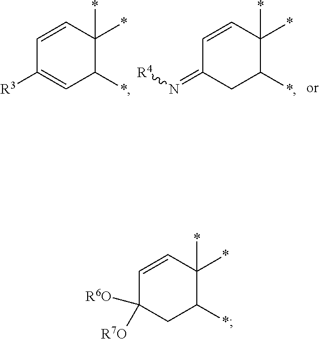

wherein * denotes the attachment points of ring A to the compound of formula (I), and wherein
   R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy;
   R⁴ is —OR⁵ or —N(R⁵)₂;
   each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl; and
   each of R⁶ and R⁷ is independently C₁-C₃ alkyl.

In certain embodiments, the compound is of formula (I):

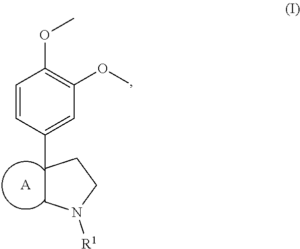

or a pharmaceutically acceptable salt thereof, wherein
R¹ is methyl; and
ring A is

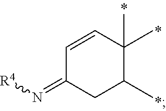

wherein * denotes the attachment points of ring A to the compound of formula (I), and wherein
   R⁴ is —OR⁵ or —N(R⁵)₂; and
   each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl.

In certain embodiments, the compound is of formula (I):

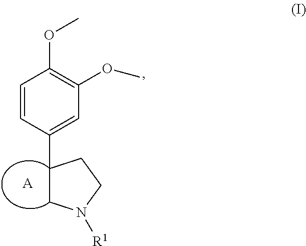

or a pharmaceutically acceptable salt thereof, wherein
R¹ is methyl; and
ring A is

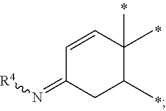

wherein * denotes the attachment points of ring A to the compound of formula (I), and wherein
   R⁴ is —OR⁵ or —N(R⁵)₂; and
   each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl.

In certain embodiments, the compound is of formula (I):

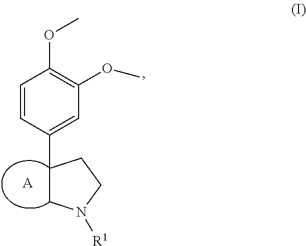

or a pharmaceutically acceptable salt thereof, wherein
R¹ is methyl; and
ring A is

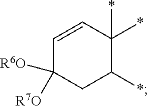

wherein * denotes the attachment points of ring A to the compound of formula (I), and
   each of R⁶ and R⁷ is independently C₁-C₃ alkyl.

In certain embodiments, the compound is of formula (V):

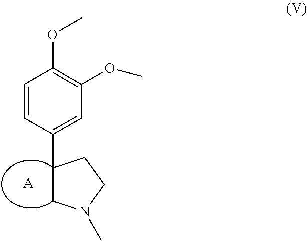

or a pharmaceutically acceptable salt thereof,
wherein
ring A is

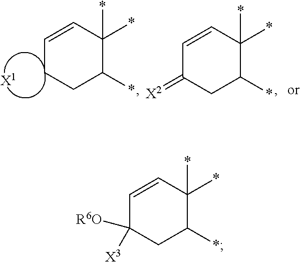

wherein * denotes the attachment points of ring A to the compound of formula (I), and wherein
   each of W and Z is independently O, NH, or S;
R⁶ is C₁-C₃ alkyl;
X¹ is >L-R¹⁴, wherein L is absent or a linker and where > denotes the two single bonds to the cyclohexane ring such that the ring containing X¹ forms a C₃-C₆ alkyl ring, which is optionally substituted by C₁-C₃ alkanol;
X² is =L-R¹⁴, wherein L is absent or a linker;
X³ is -L-R¹⁴, wherein L is absent or a linker; and
R¹⁴ comprises a generally recognized as safe (GRAS) compound.

In certain embodiments, the compound is of formula (V):

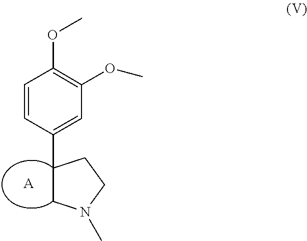

or a pharmaceutically acceptable salt thereof,
wherein
ring A is

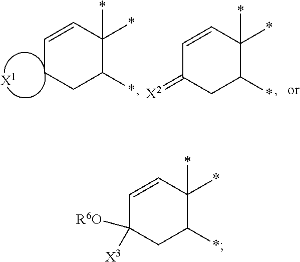

wherein * denotes the attachment points of ring A to the compound of formula (I), and wherein
R⁶ is C₁-C₃ alkyl;
X¹ is >L-R¹⁴, wherein L is absent or a linker and where > denotes the two single bonds to the cyclohexane ring such that the ring containing X¹ forms a C₃-C₆ alkyl ring, which is optionally substituted by C₁-C₃ alkanol;
X² is =L-R¹⁴, wherein L is absent or a linker;
X³ is -L-R¹⁴, wherein L is absent or a linker; and
wherein -L-R¹⁴ is selected from

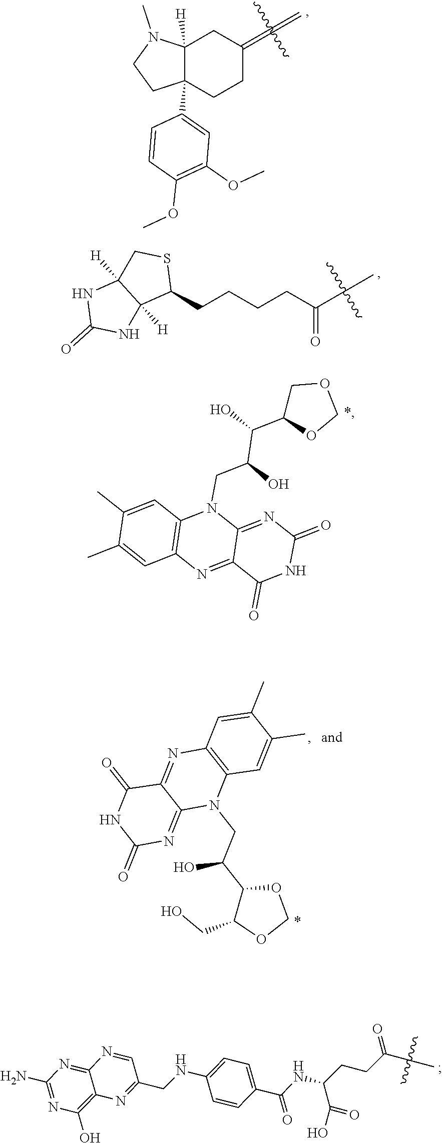

and
the * denotes the carbon of the cyclohexyl ring when L is absent.

In certain embodiments, the compound is of formula (V):

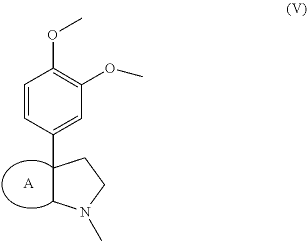

or a pharmaceutically acceptable salt thereof,
wherein
ring A is

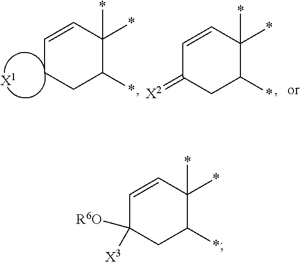

wherein * denotes the attachment points of ring A to the compound of formula (I), and wherein
R⁶ is C₁-C₃ alkyl;
X¹ is >L-R¹⁴, wherein L is absent or a linker and where > denotes the two single bonds to the cyclohexane ring such that the ring containing X¹ forms a C₃-C₆ alkyl ring, which is optionally substituted by C₁-C₃ alkanol;
X² is =L-R¹⁴, wherein L is absent or a linker;
X³ is -L-R¹⁴, wherein L is absent or a linker; and
wherein -L-R¹⁴ is selected from

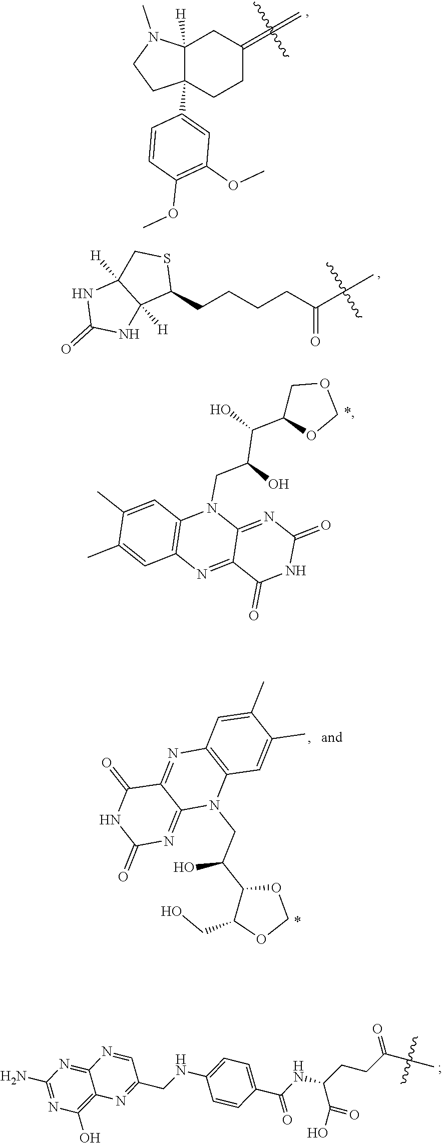

and
the * denotes the carbon of the cyclohexyl ring when L is absent.

In certain embodiments, the compound is of formula (V):

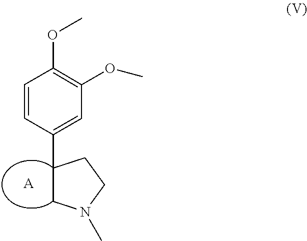

or a pharmaceutically acceptable salt thereof,
wherein
ring A is

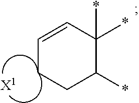

wherein * denotes the attachment points of ring A to the compound of formula (I), and wherein
X¹ is >L-R¹⁴, wherein L is absent or a linker and where > denotes the two single bonds to the cyclohexane ring such that the ring containing X¹ forms a C₃-C₆ alkyl ring, which is optionally substituted by C₁-C₃ alkanol.

### EXAMPLES

LC/MS spectra were obtained using Agilent 1200\G1956A or SHIMADZU LCMS-2020. Standard LC/MS conditions were as follows (running time 1.55 minutes):

Acidic condition: Mobile Phase A: 0.0375% TFA in water (v/v). Mobile Phase B: 0.01875B TFA in acetonitrile (v/v); Column: Kinetex EVO C18 30*2.1 mm, 5 μm.

Basic condition: Mobile Phase A: 0.0250 NH₃·H₂O in water (v/v). Mobile Phase B: Acetonitrile; Column: Kinetex EVO C18 2.1×30 mm, 5 μm.

5-95AB_0.8 min

| Instrument | | SHIMADZU LCMS-2020; | | |
|---|---|---|---|---|
| Software | | LabSolution Version 5.97SP1 | | |
| HPLC | Column | Kinetex ® EVO C18 2.1 × 30 mm 5 um | | |
| | Mobile Phase | A: 0.0375% TFA in water (v/v) | | |
| | | B: 0.01875% TFA in Acetonitrile (v/v) | | |
| | | Time(min) | B(%) | Flow(mL/min) |
| | Gradient | 0.00 | 5.0 | 2.0 |
| | | 0.60 | 95.0 | 2.0 |
| | | 0.78 | 95.0 | 2.0 |
| | | 0.79 | 5.0 | 2.0 |
| | | 0.80 | 5.0 | 2.0 |

| | | |
|---|---|---|
| | Column Temp | 50° C. |
| | Detector | PDA (220 nm&254 nm) |
| MS | Ionization source | ESI |
| | Drying Gas | N2 |
| | Drying Gas Flow | 15(L/min) |
| | DL Voltage | 120(v) |
| | Qarray DC Voltage | 20(V) |
| | MS Polarity | Positive |
| | MS Mode | Scan |
| | Mass range | 100-1000 |

Table of Abbreviations

| | |
|---|---|
| Ac | Acetyl |
| Et | Ethyl |
| Me | Methyl |
| Ms | Methanesulfonyl |
| Ts | p-Toluenesulfonyl |
| Tol | toluene |
| HPLC | High Performance Liquid Chromatography |
| DMSO | Dimethyl sulfoxide |
| LC-MS | Liquid chromatography - mass spectrometry |
| NMR | Nuclear magnetic resonance |
| m | multiplet |
| d | Doublet |
| s | singlet |
| t | triplet |
| SFC | Supercritical fluid chromatography |
| IPA | isopropanol |
| ESI | Electrospray ionization |
| EtOAC | Ethyl acetate |
| t-buOK | Potassium tertbutoxide |
| ACN | acetonitrile |
| Ac | acetate |
| MS | Molecular sieves |
| DCM | dichloromethane |
| TMS | trimethylsilyl |

### Summary of Mesembrenone Compound Designations

| Compound | Racemic | (−) enantiomer | (+) enantiomer |
|---|---|---|---|
| Mesembrenone | Compound 016 | Compound 003 | Compound 004 |

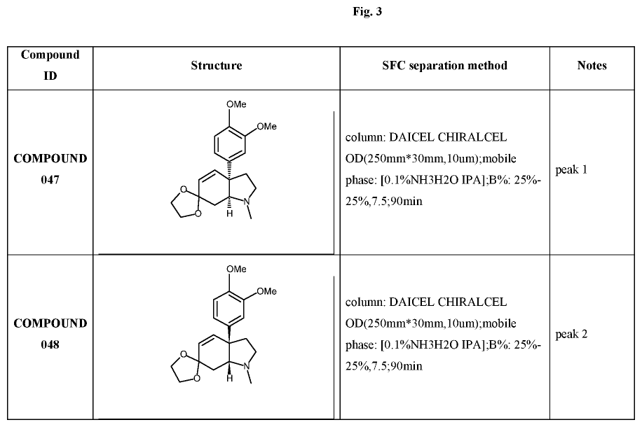

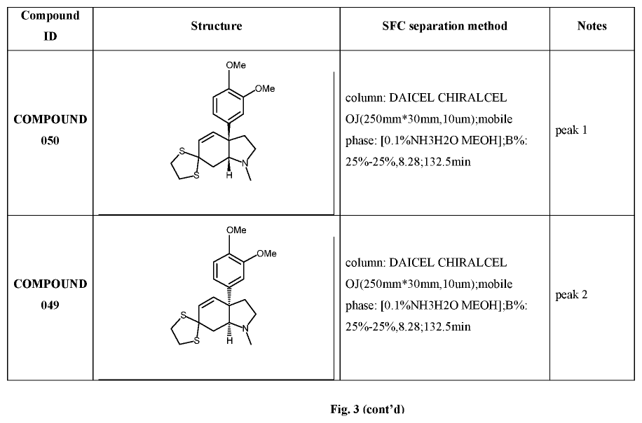

The synthesis of certain exemplary compounds is described below. In addition to the synthetic methods provided in the Examples, FIG. 3 is a table of SFC separation methods used to separate certain compounds, and FIG. 4 is a table describing purification methods used to separate certain compounds.

### Example 1: Synthesis of 015, Including Compounds 047 and 048

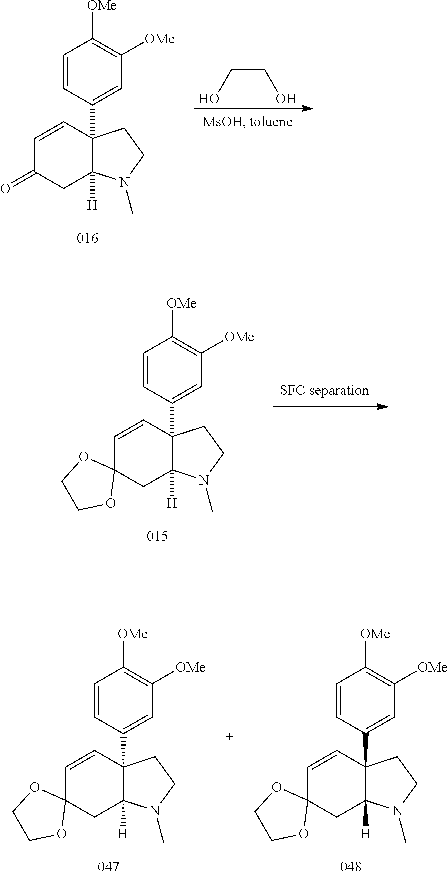

### Step 1 (3′aR,7′aS)-3′a-(3,4-dimethoxyphenyl)-1′-methyl-spiro[1,3-dioxolane-2,6′-2,3,7,7a-tetrahydroindole] (015)

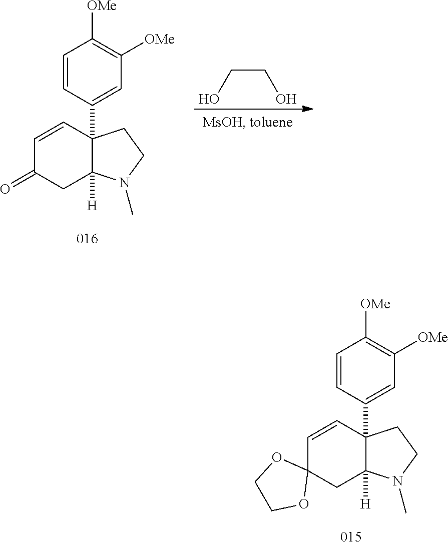

A mixture of ethylene glycol (1.08 g, 17.4 mmol, CAS #107-21-1), 3a-(3,4-dimethoxyphenyl)-1-methyl-2,3,7,7a-tetrahydroindol-6-one (200 mg, 696 umol, from 016), methanesulfonic acid (33.4 mg, 348 umol, 24.7 uL) in toluene (10 mL) was degassed and purged with N₂ for 3 times, and then the mixture was stirred at 120° C. for 10 hours under N₂ atmosphere. On completion, the reaction mixture was concentrated in vacuo to give a residue. The residue was purified by normal phase HPLC (column: Welch Ultimate XB-NH2 250*50*10 um; mobile phase: [Hexane-EtOH]; B %: 5%-20%, 20 min) to give the title compound (200 mg) as white oil.

LC-MS (ESI+) m/z 332.3 (M+H)⁺. ¹H NMR (400 MHz, CHLOROFORM-d) δ 6.93-6.86 (m, 2H), 6.84-6.77 (m, 1H), 5.84-5.79 (m, 1H), 5.76-5.70 (m, 1H), 4.12-4.05 (m, 1H), 4.01-3.93 (m, 3H), 3.88 (d, J=9.2 Hz, 6H), 3.38 (t, J=8.2 Hz, 1H), 2.49-2.39 (m, 1H), 2.38-2.26 (m, 5H), 2.20-2.10 (m, 1H), 2.07-1.98 (m, 1H), 1.92-1.83 (m, 1H).

### Step 2 (3′aR,7′aS)-3′a-(3,4-dimethoxyphenyl)-1′-methyl-spiro[1,3-dioxolane-2,6′-2,3,7,7a-tetrahydroindole] & (3′aS,7′aR)-3′a-(3,4-dimethoxyphenyl)-1′-methyl-spiro[1,3-dioxolane-2,6′-2,3,7,7a-tetrahydroindole]] (047/048)

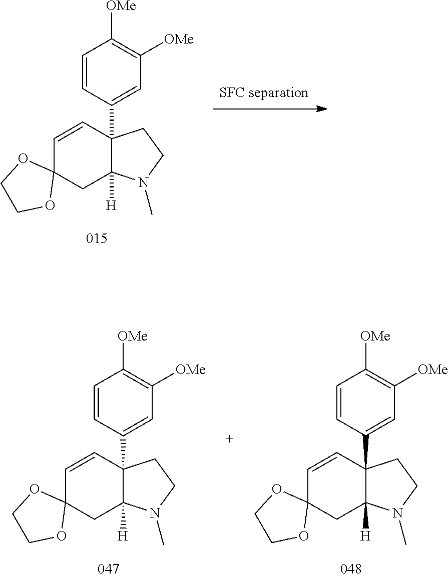 [SMILES: COc1ccc([C@@]23C=CC4(C[C@@H]2N(C)CC3)OCCO4)cc1OC ; COc1ccc([C@@]23C=CC4(C[C@@H]2N(C)CC3)OCCO4)cc1OC ; COc1ccc([C@]23C=CC4(C[C@H]2N(C)CC3)OCCO4)cc1OC]

200 mg of 015 was separated by SFC (column: DAICEL CHIRALCEL OD (250 mm*50 mm, um); mobile phase: [0.1% NH₃H₂O IPA]; B %: 25%-25%, 3.9; 40 min) to give 047 (60 mg) as yellow oil and 048 (60 mg) as yellow oil.

047:

LC-MS (EST+) m/z 332.3 (M+H)+

¹H NMR (400 MHz, CHLOROFORM-d) δ 6.94-6.88 (m, 2H), 6.85-6.78 (m, 1H), 5.84-5.77 (m, 1H), 5.75-5.68 (m, 1H), 4.12-4.05 (m, 1H), 4.00-3.93 (m, 3H), 3.88 (d, J=9.2 Hz, 6H), 3.38 (t, J=8.0 Hz, 1H), 2.44 (q, J=9.2 Hz, 1H), 2.39-2.25 (m, 5H), 2.21-2.10 (m, 1H), 2.08-1.98 (m, 1H), 1.93-1.83 (m, 1H)

048:

LC-MS (ESI+) m/z 332.3 (M+H)⁺.

¹H NMR (400 MHz, CHLOROFORM-d) δ 6.95-6.85 (m, 2H), 6.84-6.79 (m, 1H), 5.87-5.78 (m, 1H), 5.76-5.69 (m, 1H), 4.13-4.05 (m, 1H), 4.00-3.92 (m, 3H), 3.88 (d, J=9.2 Hz, 6H), 3.38 (t, J=8.4 Hz, 1H), 2.51-2.40 (m, 1H), 2.35 (s, 5H), 2.21-2.10 (m, 1H), 2.08-1.98 (m, 1H), 1.93-1.83 (m, 1H)

### Example 2: Synthesis of 017, Including Compounds 049 and 050

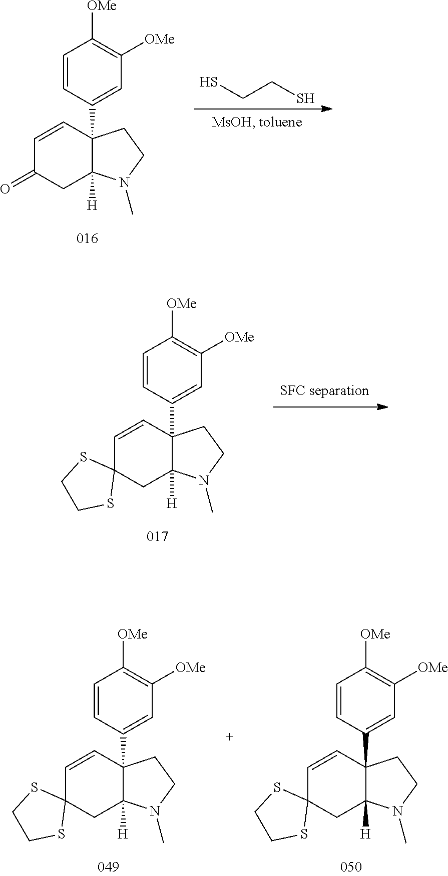

### Step 1 3′a-(3,4-dimethoxyphenyl)-1′-methyl-spiro[1,3-dithiolane-2,6′-2,3,7,7a-tetrahydroindole] (017)

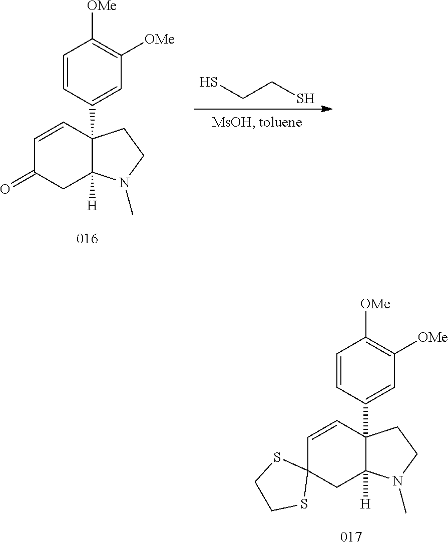

A mixture of 3a-(3,4-dimethoxyphenyl)-1-methyl-2,3,7,7a-tetrahydroindol-6-one (200 mg, 696 umol, from 016), ethane-1,2-dithiol (1.64 g, 17.4 mmol, 1.46 mL), 4A MS (20 mg, 696 umol), MsOH (20.0 mg, 208 umol, 14.8 uL) in toluene (3 mL) was degassed and purged with N₂ for 3 times, and then the mixture was stirred at 120° C. for 8 hours under N₂ atmosphere. On completion, the reaction mixture was concentrated in vacuo to give a residue. The residue was purified by prep-HPLC (column: Phenomenex C18 250*50 mm*10 um; mobile phase: [water (ammonia hydroxide v/v)-ACN]; B %: 43%-73%,8 min) to give the title compound (50 mg) as yellow solid. LC-MS (ESI+) m/z 364.2 (M+H)⁺. ¹H NMR (400 MHz, CHLOROFORM-d) δ 6.93-6.80 (m, 3H), 5.99 (d, J=10.0 Hz, 1H), 5.47 (d, J=10.0 Hz, 1H), 3.90 (d, J=8.4 Hz, 6H), 3.56-3.18 (m, 5H), 3.09-2.92 (m, 1H), 2.56-2.46 (m, 1H), 2.45-2.38 (m, 4H), 2.38-2.32 (m, 1H), 2.30-2.21 (m, 1H), 2.12-2.02 (m, 1H).

### Step 2 (3′aR,7′aS)-3′a-(3,4-dimethoxyphenyl)-1′-methyl-spiro[1,3-dithiolane-2,6′-2,3,7,7a-tetrahydroindole] & (3′aS,7′aR)-3′a-(3,4-dimethoxyphenyl)-1′-methyl-spiro[1,3-dithiolane-2,6′-2,3,7,7a-tetrahydroindole] (049 & 050)

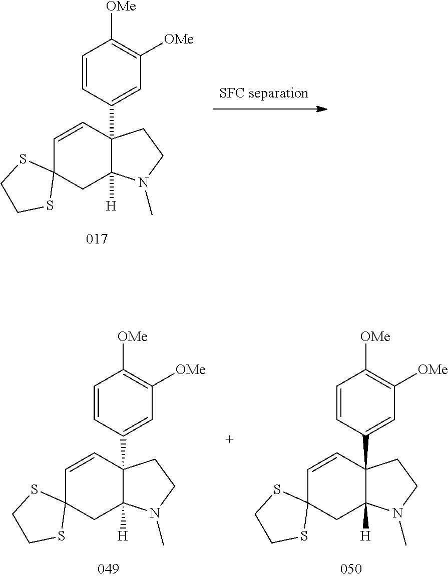 [SMILES: COc1ccc([C@@]23C=CC4(C[C@@H]2N(C)CC3)SCCS4)cc1OC ; COc1ccc([C@@]23C=CC4(C[C@@H]2N(C)CC3)SCCS4)cc1OC ; COc1ccc([C@]23C=CC4(C[C@H]2N(C)CC3)SCCS4)cc1OC]

50 mg of 017 was separated by SFC (column: DAICEL CHIRALCEL OJ (250 mm*30 mm, 10 um); mobile phase: [0.1% NH₃H2o MEOH]; B %: 25%-25%, 8.28; 132.5 min) to give 049 (20 mg, peak 2) as yellow solid and 050 (20 mg, peak 1) as yellow solid.

050:

LC-MS (ESI+) m/z 364.2 (M+H)⁺. ¹H NMR (400 MHz, CHLOROFORM-d) δ 6.92-6.85 (m, 2H), 6.85-6.79 (m, 1H), 5.97 (d, J=10.0 Hz, 1H), 5.46 (dd, J=1.2, 10.0 Hz, 1H), 3.88 (d, J=8.4 Hz, 6H), 3.45-3.22 (m, 5H), 2.48-2.42 (m, 1H), 2.42-2.37 (m, 4H), 2.36-2.30 (m, 1H), 2.28-2.15 (m, 1H), 2.05 (td, J=8.8, 13.2 Hz, 1H), 1.36-1.17 (m, 1H).

049:

LC-MS (ESI+) m/z 364.2 (M+H)⁺. ¹H NMR (400 MHz, CHLOROFORM-d) δ 6.93-6.80 (m, 3H), 5.97 (d, J=10.0 Hz, 1H), 5.46 (d, J=10.0 Hz, 1H), 3.88 (d, J=8.4 Hz, 6H), 3.46-3.21 (m, 5H), 2.56-2.43 (m, 2H), 2.42-2.30 (m, 4H), 2.30-1.96 (m, 2H), 1.40-1.21 (m, 1H).

### Example 3: Synthesis of Compound 66

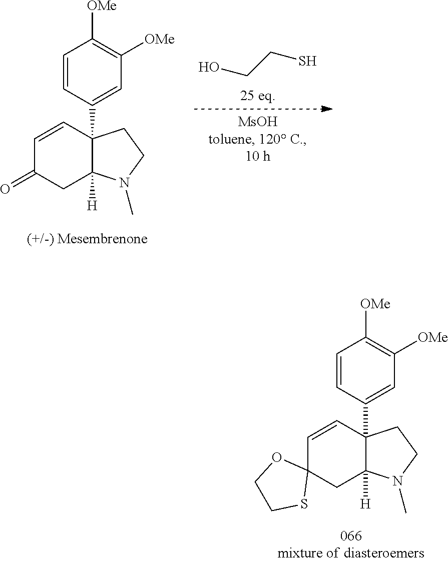

A mixture of 3a-(3,4-dimethoxyphenyl)-1-methyl-2,3,7,7a-tetrahydroindol-6-one (from 016), and 2-mercaptoethanol, 4A MS, and MsOH in toluene (3 mL) will be degassed and purged with N₂ for 3 times. The mixture will then be stirred at 120° C. for 10 hours under N₂ atmosphere. On completion, the reaction mixture will be concentrated in vacuo to give a residue. The residue will be purified by prep-HPLC (column: Phenomenex C18 250*50 mm*10 um; mobile phase: [water (ammonia hydroxide v/v)-ACN]; B %: 43%-73%,8 min) to give the title compound.

### Example A1: Hydrolysis Assay

50 uL of compound (10 mM) in DMSO was diluted into 950 uL of HCl aqueous solution (0.01 M) to a final concentration of 0.5 mM. The hydrolysis kinetics was under a certain temperature and measured by LCMS at a certain time interval. The corresponding LCMS chromatograms were recorded and the conversions were calculated. The hydrolysis conditions and the data were summarized in below table. LCMS spectra were obtained using Agilent 1200\G1956A or SHIMADZU LCMS-2020. Standard LCMS conditions were as follows (running time 1.55 minutes):

Acidic condition: Mobile Phase A: 0.0375% TFA in water (v/v). Mobile Phase B: 0.01875% TFA in acetonitrile (v/v); Column: Kinetex EVO C18 30*2.1 mm, 5 μm.

Basic condition: Mobile Phase A: 0.025% NH₃·H₂O in water (v/v). Mobile Phase B: Acetonitrile; Column: Kinetex EVO C18 2.1×30 mm, 5 μm.

Conversion (%)=peak area 1/(peak area 1+peak area 2)*100%, peak area 1 is the integration of hydrolyzed product, peak area 2 is the integration of starting material. Results are shown in Table 6.

TABLE 6

| | | | | Conver- | |
|---|---|---|---|---|---|
| Compound | | | | sion | |
| ID | Conditions | Time | | (%) | Notes |
| 017 | pH = 2.0, 37° C., | 0-24 | (h) | 0 | Stable |
| | 0.5 mM, 0.01M HCl | | | | |
| | pH = 2.0, 25° C., | 0-24 | (h) | 0 | Stable |
| | 0.5 mM, 0.01M HCl | | | | |
| 015 | pH = 2.0, 25° C., | 1 | (min) | 19 | |
| | 0.5 mM, 0.01M HCl | 10 | (min) | 46 | |
| | | 30 | (min) | 66 | |
| | | 120 | (min) | 99 | |
| 016 | PH = 7.4, 25° C., | 0-24 | (h) | 0 | Stable |

0.5 mM, PBS (20 mM)

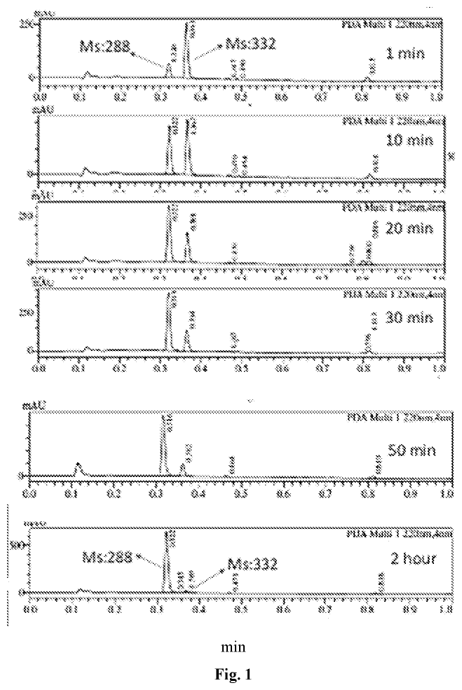

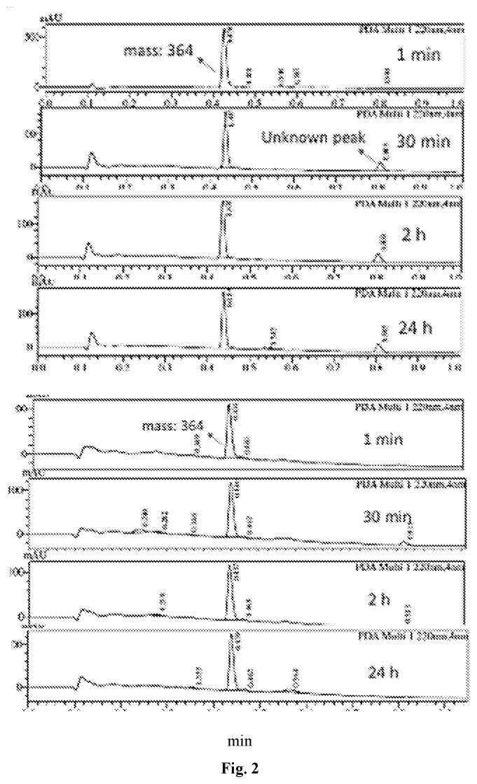

FIG. 1 is a series of LCMS graphs obtained from 015 in the acid hydrolysis assay of Example A1. FIG. 2 is a series of LCMS graphs obtained from 017 in the acid hydrolysis assay of Example A1.

### Example A2: Hydrolysis Assay

### General Method Description of Hydrolysis Assay—Condition 1 (SGF)

50 uL of compound (10 mM) in DMSO is diluted into 950 uL of Simulated Gastric Fluid (SGF) with pepsin (pH 1.5) to a final concentration of 0.5 mM. The hydrolysis kinetics are conducted at 37° C. and measured by LCMS at a certain time interval.

### General Method Description of Hydrolysis Assay—Condition 2 (DI Water)

50 uL of compound (10 mM) in DMSO is diluted into 950 uL of DI water to a final concentration of 0.5 mM. The hydrolysis kinetics are conducted at 25° C. and measured by LCMS at a certain time interval.

The corresponding LCMS chromatogram are recorded and the conversions are calculated by integration of each peak.

Note:
1) LCMS spectra are obtained using Agilent 1200G1956A or SHIMADZU LCMS-2020. Standard LCMS conditions are as follows (running time 1.55 minutes):
   Acidic condition: Mobile Phase A: 0.0375% TFA in water (v/v). Mobile Phase B: 0.01875% TFA in acetonitrile (v/v); Column: Kinetex EVO C18 30*2.1 mm, 5 μm.
   Basic condition: Mobile Phase A: 0.025% NH₃·H₂O in water (v/v). Mobile Phase B: Acetonitrile; Column: Kinetex EVO C18 2.1×30 mm, 5 μm.
2) Conversion (%)=peak area 1/(peak area 1+peak area 2)*100%, peak area 1 is the integration of hydrolyzed product, peak area 2 is the integration of starting material.

### Example B1: SERT Inhibition Assay

SERT inhibition was measured using a Neruotransmitter Transportation Fluorescence assay. Briefly, stable SHHH cells were prepared in a 384 microwell plate. Compounds were prepared by in assay buffer (20 mM HEPES, 0.1% BSA). The compounds were added to the plated cells and incubated for 30 minutes at 37° C. 25 μL of dye solution (Molecular Devices Neurotransmitter Transporter Uptake Assay Kit) was added per well and incubated for 30 minutes at 37° C. The plates were then read on a plate reader.

The in vitro SERT inhibition was measured for the compounds listed in the table below.

TABLE 7

| Compound | SERT IC50 (nM) |
|---|---|
| 016 | 206 |
| 047 | 581 |
| 048 | 939 |
| 049 | 159 |
| 050 | 405 |

### Example B2: Pharmacokinetics of 047 and 016

Total twelve male mice were used in this study. Animals were administered through oral route with solution formulation of 047 at 75 mg/kg dose. The formulation vehicle used was normal saline. Blood samples (approximately 60 μL) were collected under light isoflurane anesthesia from a set of two mice at 0.25, 0.5, 1, 2, 4 and 8 h. The blood samples were collected at each time point in labeled micro centrifuge tube containing K₂EDTA as anticoagulant and PMSF (100 mM; 10 μL/mL of blood) as a stabilizer. Plasma samples were separated by centrifugation of whole blood and stored below −70° C. until bioanalysis. Following blood collection, immediately animals were sacrificed followed by abdominal vena-cava was cut open and whole body was perfused from heart using 10 mL of normal saline. Brain samples were collected from set of two mice at 0.25, 0.5, 1, 2, 4 and 8 h. After isolation, brain samples were rinsed three times in ice cold normal saline (for 5-10 seconds/rinse using ˜10-20 mL normal saline in disposable petri dish for each rinse) and dried on blotting paper. Brain samples were homogenized using ice-cold phosphate buffer saline (pH-7.4) and PMSF (100 mM; 10 μL/mL of blood) as a stabilizer. Brain homogenates were stored below −70±10° C. until analysis. Total homogenate volume was three times of the brain weight.

The plasma and brain concentration-time data of 047 and 016 was used for the pharmacokinetic analysis. Plasma and brain samples were quantified by fit-for-purpose LC-MS/MS method.

LLOQ: 047-02N: 1.01 ng/mL for plasma and brain; 016: 2.04 ng/mL for plasma and brain.

Following a single oral at 75 mg/kg dose administration of 047 in male C57BL/6 Mice, peak plasma concentrations were observed at 0.25 h, suggesting rapid absorption. Brain concentrations were quantifiable up to 4 h with brain exposure-Kp was 1.05.

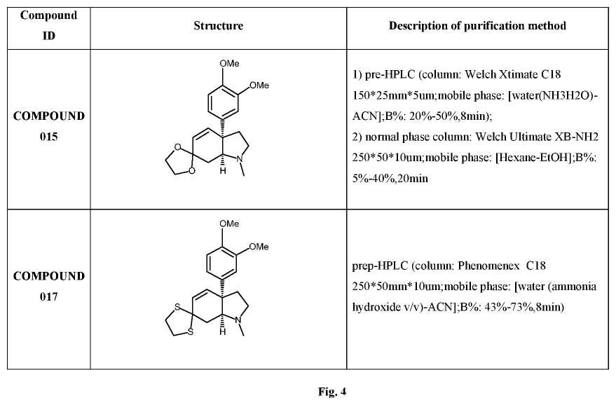

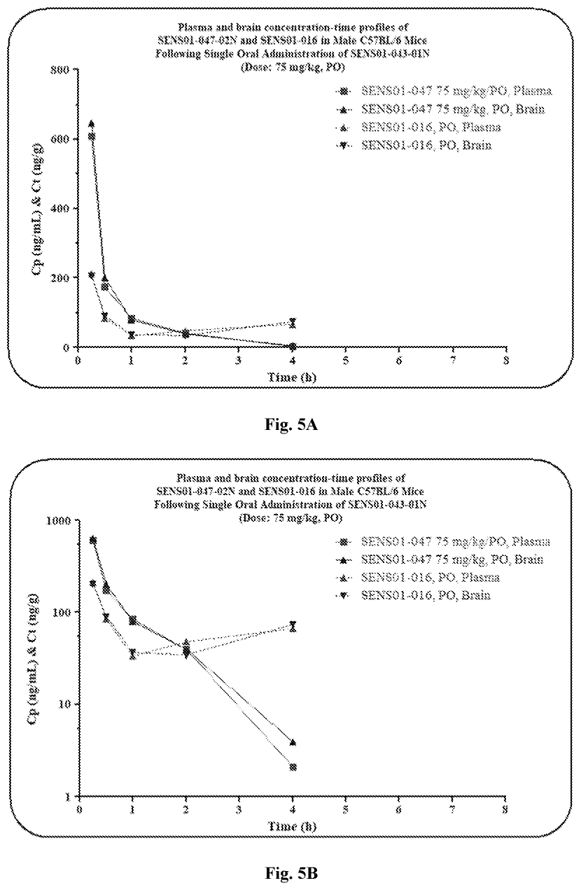

Following a single oral at 75 mg/kg dose administration of 047 in male C57BL/6 Mice, peak plasma concentrations of 016 were observed at 0.25 h, suggesting rapid absorption of metabolite. Brain concentrations were quantifiable up to 4 h with brain exposure-Kp was <1. The results are summarized in Table 8 and shown in FIGS. 5A and 5B.

Pharmacokinetic data of 047 and 016 in male C57BL/6 mice following a single oral administration of 047 (Dose: 75 mg/kg, PO)

TABLE 8

| | | | Dose | Tₘₐₓ | Cₘₐₓ | AUCₗₐₛₜ | | |
|---|---|---|---|---|---|---|---|---|
| Analyte | Matrix | Route | (mg/kg) | (h) | (ng/mL) | (h*ng/mL) | — | — |
| 047 | Plasma | PO | 75 | 0.25 | 608.60 | 342.74 | | — |
| 016 | Plasma | PO | — | 0.25 | 211.91 | 248.18 | | |
| | | | Dose | Tₘₐₓ | Cₘₐₓ | AUCₗₐₛₜ | Brain-Kp | Brain-Kp |
| | Matrix | Route | (mg/kg) | (h) | (ng/mL) | (h*ng/mL) | (Cₘₐₓ) | (AUCₗₐₛₜ) |
| 047 | Brain | PO | 75 | 0.25 | 646.55 | 360.50 | 1.06 | 1.05 |
| 016 | Brain | PO | — | 0.25 | 204.96 | 237.85 | 0.97 | 0.96 |

1-84. (canceled)

85. A compound of formula (I): 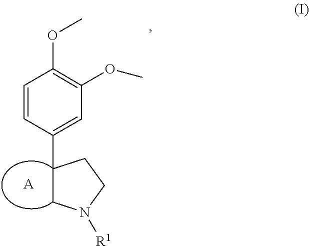
or a pharmaceutically acceptable salt thereof, wherein:
R¹ is C₁-C₇ alkyl or H; and
ring A is 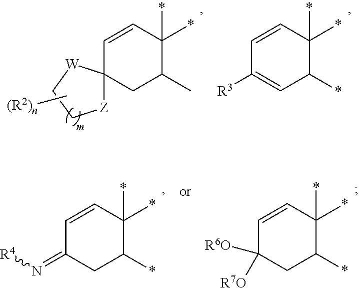
wherein* denotes the attachment points of ring A to the compound of formula (I), and wherein
each of W and Z is independently O, NH, or S;
each R² is independently C₁-C₃ alkyl, C₂-C₆ alkenyl, —COOH, —CONH₂, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol;
m is 1 or 2;
n is 0, 1, 2, or 3;
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁O cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy;
R⁴ is —OR⁵ or —N(R⁵)₂;
each R⁵ is H, C₁-C₆ alkyl, C₃-C₆ heterocycloalkyl, C₃-C₁O cycloalkyl, C₁-C₆ alkoxy, phenyl, 5- to 10-membered heteroaryl, —C(O)-5- to 7-membered heteroaryl, —C(O)—C₁-C₈alkyl, —C(O)—C₃-C₁O cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁O cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁O cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl; and
each of R⁶ and R⁷ is independently C₁-C₃ alkyl.

86. The compound of claim 85, wherein the compound is a compound of formula (I-1): 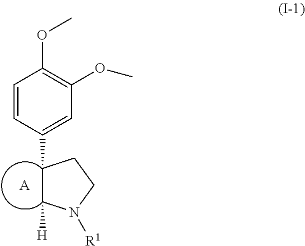
or a pharmaceutically acceptable salt thereof.

87. The compound of claim 86, wherein ring A is

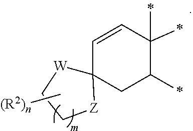

88. The compound of claim 87, wherein R¹ is methyl.

89. The compound of claim 88, wherein n is 0 and m is 1.

90. The compound of claim 89, wherein W is O and Z is O; or W is S and Z is S.

91. The compound of claim 88, wherein n is 1 and m is 1.

92. The compound of claim 91, wherein each R² is independently C₁-C₃ alkyl, C₂-C₆ alkenyl, —COOH, —CONH₂, phenyl, 5- to 6-membered heterocyclyl, or 5- to 6-membered heteroaryl, wherein each hydrogen atom in C₁-C₃ alkyl, C₂-C₆ alkenyl, phenyl, 5- to 6-membered heterocyclyl and 5- to 6-membered heteroaryl is optionally substituted by —OH, C₁-C₃ alkyl, C₁-C₃ alkoxy, C₁-C₃ alkanol, or —NO₂.

93. The compound of claim 91, wherein R² is furanone, phenyl or 6-membered heterocyclyl optionally substituted by OH, C₁-C₃ alkyl, C₁-C₃ alkoxy, C₁-C₃ alkanol, or —NO₂.

94. The compound of claim 93, wherein R² is phenyl, pyridine or pyrimidine optionally substituted with methyl, methoxy, nitro, or hydroxymethyl.

95. The compound of claim 93, wherein is

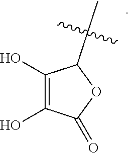 [SMILES: *C1OC(=O)C(O)=C1O]

96. The compound of claim 88, wherein two R²s on a single carbon atom combine to form ═O.

97. The compound of claim 88, wherein two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃alkanol.

98. The compound of claim 89, wherein the compound is a compound of formula (X-2) 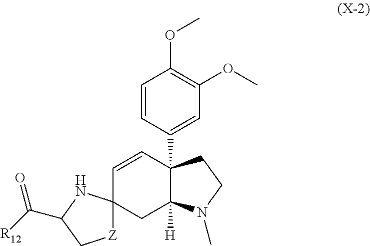
or a pharmaceutically acceptable salt thereof, wherein
Z is O or S; and
R₁₂ is an amino or hydroxyl.

99. The compound of claim 85, wherein the compound is selected from the group consisting of: 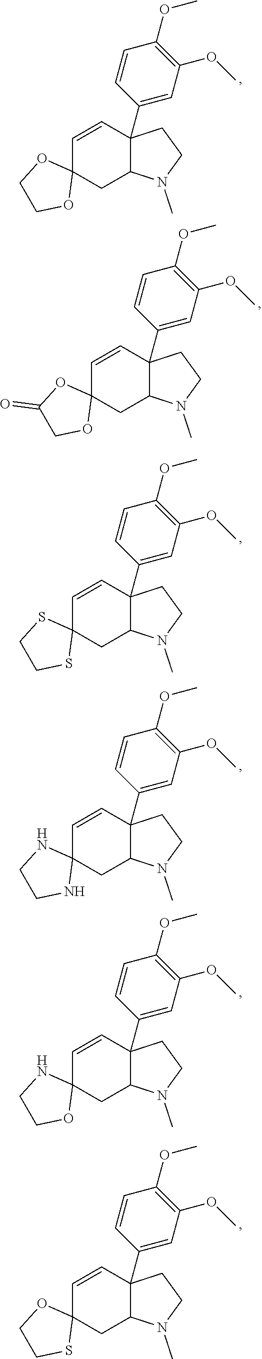 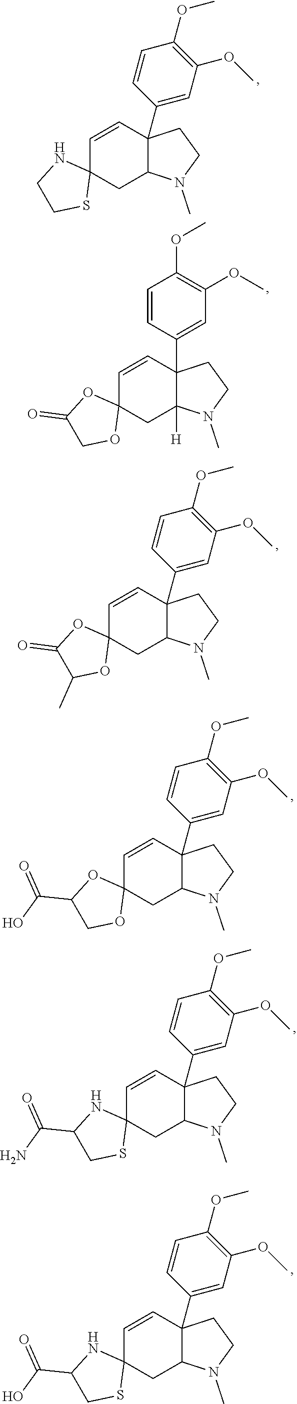 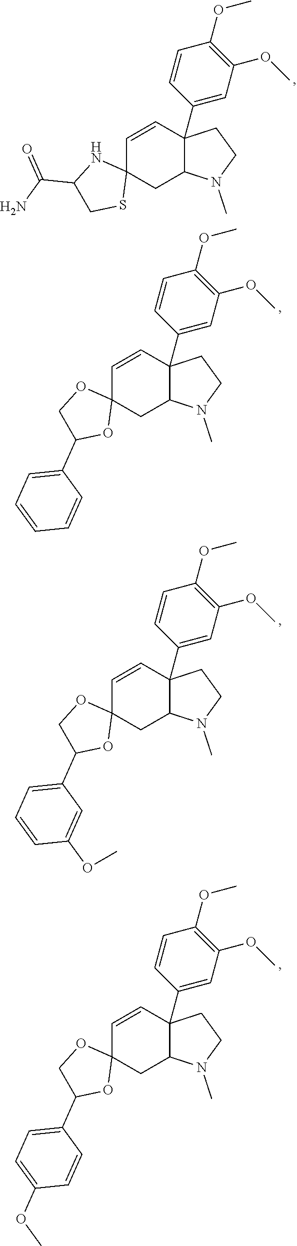 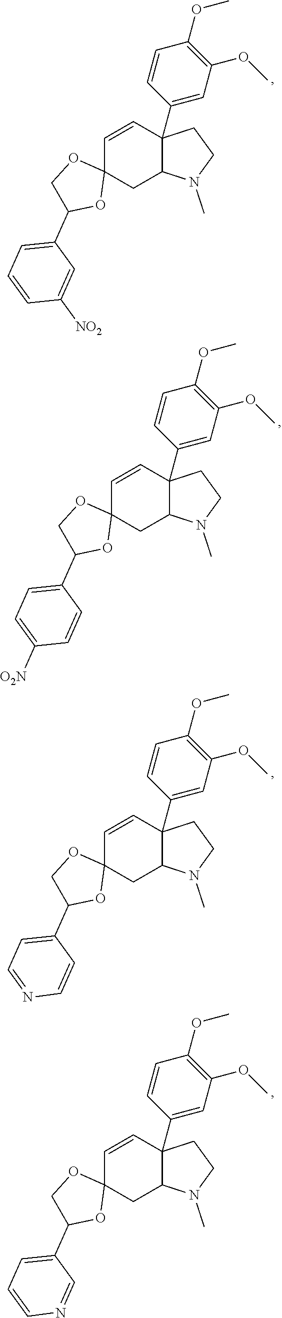 [SMILES: COc1ccc(C23C=CC4(CC2N(C)CC3)OCC(c2cccc([N+](=O)[O-])c2)O4)cc1OC ; COc1ccc(C23C=CC4(CC2N(C)CC3)OCC(c2ccc([N+](=O)[O-])cc2)O4)cc1OC ; COc1ccc(C23C=CC4(CC2N(C)CC3)OCC(c2ccncc2)O4)cc1OC ; COc1ccc(C23C=CC4(CC2N(C)CC3)OCC(c2cccnc2)O4)cc1OC] 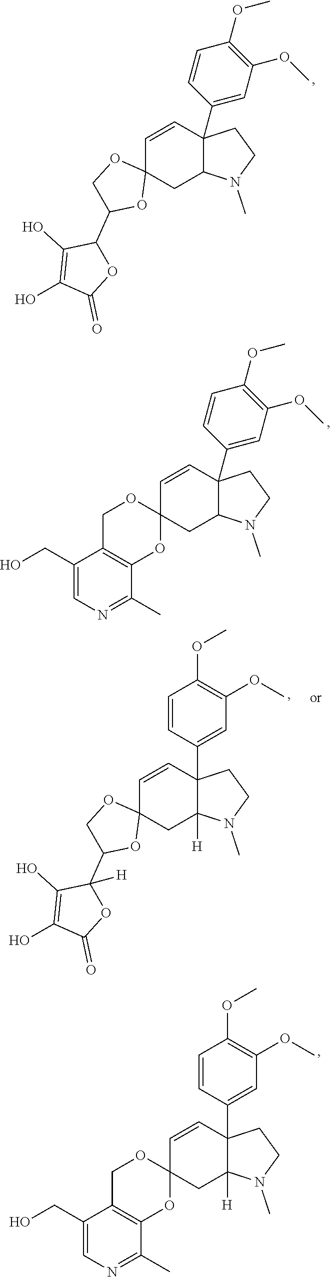 [SMILES: COc1ccc(C23C=CC4(CC2N(C)CC3)OCC(C2OC(=O)C(O)=C2O)O4)cc1OC ; COc1ccc(C23C=CC4(CC2N(C)CC3)OCc2c(CO)cnc(C)c2O4)cc1OC ; COc1ccc(C23C=CC4(CC2N(C)CC3)OCC(C2OC(=O)C(O)=C2O)O4)cc1OC ; COc1ccc(C23C=CC4(CC2N(C)CC3)OCc2c(CO)cnc(C)c2O4)cc1OC]
or a pharmaceutically acceptable salt thereof.

100. The compound of claim 85, wherein the compound is 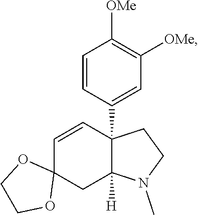 [SMILES: COc1ccc([C@@]23C=CC4(C[C@@H]2N(C)CC3)OCCO4)cc1OC]
or a pharmaceutically acceptable salt thereof.

101. The compound of claim 85, wherein the compound is 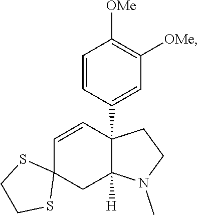
or a pharmaceutically acceptable salt thereof.

102. The compound of claim 86, wherein ring A is 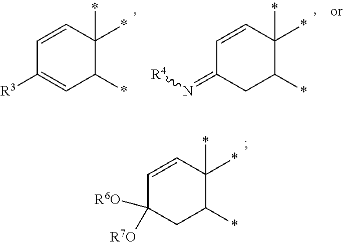
wherein * denotes the attachment points of ring A to the compound of formula (I)

103. A compound of formula (IA): 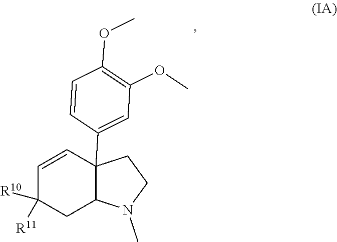
or a pharmaceutically acceptable salt thereof, wherein
R¹⁰ and R¹¹ are acid hydrolysable moieties that are hydrolysed to a ketone moiety after 24 hours at a pH of 2 (0.01 M HCl) and a temperature of 37° C. in the Hydrolysis Assay of Example A1;
wherein at least about 10% of the compound of Formula (IA) is hydrolysed to mesembrenone as measured by HPLC after 24 hours at a pH of 2.0 (0.01M HCl) and a temperature of 37° C. in the Hydrolysis Assay of Example A1.

104. A compound of formula (V): 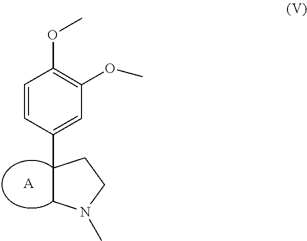
or a pharmaceutically acceptable salt thereof, wherein
ring A is 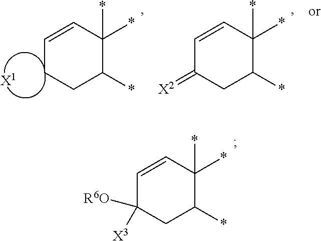
wherein * denotes the attachment points of ring A to the compound of formula (V), and wherein
R⁶ is C₁-C₃ alkyl;
X¹ is >L-R¹⁴, wherein L is absent or a linker and where > denotes the two single bonds to the cyclohexane ring such that the ring containing X¹ forms a C₃-C₆ alkyl ring, which is optionally substituted by C₁-C₃ alkanol;
X² is =L-R¹⁴, wherein L is absent or a linker;
X³ is -L-R¹⁴, wherein L is absent or a linker; and
wherein -L-R¹⁴ is selected from 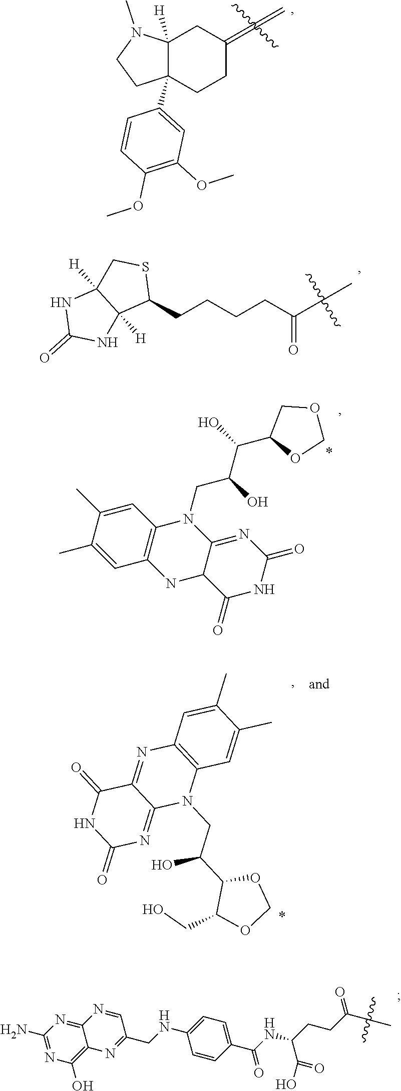
and
the * denotes the carbon of the cyclohexyl ring when L is absent.
